(19) United States
(12) Patent Application Publication
(10) Pub. No.: US 2022/0233488 A1
(43) Pub. Date: Jul. 28, 2022

(54) VERY LONG CHAIN FATTY ACIDS FOR TREATMENT AND ALLEVIATION OF DISEASES

(71) Applicant: EPAX NORWAY AS, Ålesund (NO)

(72) Inventors: Iren Merete Skjåstad STOKNES, Ålesund (NO); Harald BREIVIK, Inndyr (NO); Harald SVENSEN, Ålesund (NO)

(21) Appl. No.: 17/614,751

(22) PCT Filed: May 29, 2020
(86) PCT No.: PCT/NO2020/050141

§ 371 Date: Nov. 29, 2021

(30) Foreign Application Priority Data
May 31, 2019 (NO) 20190689

(51) Int. Cl. A61K 31/202; A61K 31/201

(52) U.S. Cl. CPC A61K 31/202; A61K 31/201

(57) ABSTRACT

The present invention relates to methods and compositions for treatment and alleviation of diseases. Particularly, the invention provides compositions comprising very long chain fatty acids for use in treatment, such as of subjects having a deficient or abnormal level of VLCFAs present in specific tissue which play a role in the disease. Particularly, the invention provides methods and compositions for treatment of subjects having a reduced ability for endogenic synthesis of fatty acids.

### FIELD OF THE INVENTION

The present invention relates to methods and compositions for treatment and alleviation of diseases. Particularly, the invention provides a method for treatment of diseases associated with a reduced ability for endogenic synthesis of fatty acids.

### BACKGROUND OF THE INVENTION

Among the long-chain polyunsaturated fatty acids (LCPUFAs), and especially long-chain omega-3 fatty acids (LCn3), the fatty acids of chain length C20-C22 have received most interest in literature. The acronyms EPA (for eicosapentaenoic acid) and DHA (for docosahexaenoic acid) have become household names in describing valuable omega-3-acids from fish oil and other sources. Products rich in alpha-linolenic acid (ALA) from plant sources are also available in the market.

More recently, the long-chain monounsaturated fatty acids (LCMUFAs) with chain length C20-C22 have come into the focus of scientific interest. See, for example, Breivik and Vojnovic, Long chain monounsaturated fatty acid composition and method for production thereof, U.S. Pat. No. 9,409,851B2.

In this regard, it is noted that lipids are described by the formula X:YnZ wherein X is the number of carbon atoms in their alkyl chain, and Y is the number of double bonds in such chain; and where “nZ” is the number of carbon atoms from the methyl end group to the first double bond. In nature the double bonds are all in the cis-form. In polyunsaturated fatty acids each double bond is separated from the next by one methylene (—CH₂) group. Using this nomenclature, EPA is C20:5n3; DHA is C22:6n3 and ALA is C18:3n3. Further, natural sources of omega-3 fatty acids, such as fish oil, also comprise fatty acids of shorter and longer length than C20-C22.

In order to produce marine omega-3-concentrates rich in EPA and DHA, conventional industrial processes are designed to concentrate the C20-C22 fraction, by removing both short-chain fatty acids as well as larger molecules than the C22 fatty acids. Examples of such processes are molecular/short path distillation, urea fractionation, extraction and chromatographic procedures, all of which can be utilized to concentrate the C20-22 fraction of marine fatty acids and similar materials derived from other sources. A review of these procedures is provided in Breivik H (2007) Concentrates. In: Breivik H (ed) Long-Chain Omega-3 Specialty Oils. The Oily Press, P J Barnes & Associates, Bridgwater, UK, pp 111-140. In addition to the omega-3 acids, the polyunsaturated fatty acids of marine oils can contain smaller amounts of omega-6 fatty acids.

For important fish sources, like North Atlantic herring and mackerel, the C20-C22 fatty acid fraction, in addition to omega-3-acids like EPA and DHA, also contains substantial amounts of C20-C22 MUFAs. A procedure for separation of C20-C22 MUFAs and PUFAs is disclosed in U.S. Pat. No. 9,409,851B2.

Omega-3-acids are very liable to oxidation. In order to comply with pharmacopoeia and voluntary standards imposing upper limits for oligomeric/polymeric oxidation products, it is common to remove components with chain lengths above that of DHA, for example by distillation, extraction and similar procedures. Further, such higher molecular weight components of marine oils are typically associated with undesirable unsaponifiable constituents of such oil including cholesterol as well as with organic pollutants such as brominated diphenyl ethers.

Omega-3 fatty acids, and particularly the LCPUFAs EPA, DHA and n3DPA (n3 docosapentaenoic acid, C22:5n3), are known to have a broad range of beneficial health effects and are hence known for different uses. These LC omega-3 fatty acids are naturally found in fish and other marine organisms. They can also be derived in the body from ALA, an omega-3 fatty acid which is found in certain plant- and animal-based oils. However, the body is insufficient in converting ALA into LC omega-3 acids. For this reason, LC omega-3-acids are often referred to as “essential” fatty acids. Fatty acids are taken up by cells, where they may serve as precursors in the synthesis of other compounds, as fuels for energy production, and as substrates for ketone body synthesis. In addition, some cells synthesize fatty acids for storage or export. Fatty acids taken by a subject, such as from dietary sources, are often modified in vivo. Such modifications may include chain elongation to give longer fatty acids and/or desaturation, giving unsaturated fatty acids.

It is well known that subjects may experience disorders of fatty acid metabolism, and this can be described in terms of, for example, hypertriglyceridemia (too high level of triglycerides), or other types of hyperlipidemia. These may be familial or acquired. These disorders may be described as fatty oxidation disorders or as a lipid storage disorders, and are any one of several inborn errors of metabolism that result from enzyme defects affecting the ability of the body to oxidize fatty acids in order to produce energy within muscles, liver, and other cell types. Further, in addition to disorders associated with the metabolism of fatty acids, some subjects may experience reduced ability for endogenic synthesis of fatty acids, as they, for example, have a reduced ability to synthesize longer fatty acids from shorter fatty acids. Thus, these subjects may have a reduced ability for endogenic synthesis of long chain fatty acids from fatty acids of a shorter length. Such reduced ability for endogenic synthesis may be in specific tissues where these fatty acids are needed for maintaining the subjects' optimal health. The reduced ability may develop with age or may be present already at young age. Especially in the latter case, reduced ability for endogenic synthesis of longer fatty acids may be caused by hereditary diseases.

Supplements containing concentrates of traditional C20-C22 omega-3 fatty acids are often recommended in order to treat or alleviate symptoms of different conditions. Also diseases and conditions like age-related macular degeneration (AMD), dry eye disease (DED), reduced mental health and reduced sperm quality of male subjects have been treated with traditional C20-C22 omega-3 fatty acids, such as those comprising a high concentration of

EPA and/or DHA. However, not all subjects respond to such treatment satisfactory, and results can appear conflicting, depending on whether the subjects consume omega-3 fatty acids by eating a fish rich diet, or by taking traditional C20-C22 concentrates. As an example, a recent publication (Gorusupudi A, Liu A, Hageman GS and Bernstein P (2016) Associations of human retinal very long-chain polyunsaturated fatty acids with dietary lipid biomarkers. Journal of Lipid Research 57: 499-508) presents an unsolved paradox: While multiple epidemiological studies indicate that diets rich in n3 LCPUFAs are associated with lower risk of AMD, two clinical trials with 3-5 years of “fish oil” supplementations have failed to show any impact on progression to advanced AMD.

An explanation of this paradox might be the incorrect assumption that very long chain polyunsaturated fatty acids (VLCPUFAs) are not normally consumed in the human diet. As shown by Breivik and Svensen, WO2016/182452, oil from wild fish contains VLCPUFAs with chain length C24 and above. On the other hand, dietary “fish oil” omega-3 supplements are very often manufactured by concentrating the valuable long-chain marine omega-3 fatty acids, reducing the amount of fatty acids with shorter chain length than EPA (C20) and longer chain length than n3DPA and DHA (C22).

Similar to the paradox derived from the publication of Gorusupudi et al. for AMD, as discussed above, supplementation of omega-3-acids to patients suffering from dry eye disease (DED) has given conflicting results. DED, also known as keratoconjunctivitis sicca (KCS), is a common chronic condition that is characterised by ocular discomforts and visual disturbances that decrease quality of life. As recently described by the Dry Eye Assessment and Management (DREAM) Study Research Group (New England Journal of Medicine, Apr. 13, 2018, DOI: 10.1056/NEJMoa1709691), many clinicians recommend the use of omega-3 fatty acids to relieve symptoms of DED. However, the large DREAM Study concluded that among patients with DED, those who received supplements as omega-3 concentrates (daily intake of 3000 mg n3 fatty acids as 2000 mg EPA and 1000 mg DHA in triglyceride form) for 12 months did not have significantly better outcomes than those who received placebo.

In contrast to this, other studies have shown positive effects on DED from fish oil. As an example, in an article listed among the references in the DREAM Study report, Deinema et al. (A randomized, double-masked, placebo controlled clinical trial of two forms of omega-3 supplements for treating dry eye disease. Ophthalmology 2017; 124: 43-52) showed significantly positive effects on DED by using non-concentrated fish oil and krill oil as omega-3 sources.

The DREAM study states that many clinicians recommend dietary supplements of omega-3 fatty acids because they have anti-inflammatory activity and are not associated with substantial side effects.

In the discussion section of a recently published meta-analysis on the efficacy of omega-3 fatty acid supplementation for treatment of DED, Giannaccare et al. (2019) Efficacy of omega-3 fatty acid supplementation for treatment of Dry Eye Disease: A meta-analysis of randomized clinical trials, Cornea 38 (5) 565-573, the authors state that the effect of both dietary consumption and supplementation of omega-3 fatty acids on signs and symptoms of DED is still dubious. However, based on their study, including 17 randomized clinical studies involving 3363 patients, the authors conclude that omega-3 fatty acid supplementation improves dry eye symptoms, tear film stability and tear production in patients with DED. On the other hand, the authors comment observed substantial heterogenicity for all their outcome variables, entailing that the results were not consistent across the studies.

As disclosed by the present invention, in some diseases, the reason for the lack of response to treatment with C20-C22 omega-3 fatty acids may be that the subject has a reduced ability for endogenic synthesis of longer fatty acids from e.g. EPA and DHA, and thus is unable to synthesise the very long omega-3 fatty acids in sufficient amounts all the way up to the chain lengths and degree of unsaturation that are required for optimal health.

Similar to what is said above for LCPUFAs, VLCPUFAs could also be referred to as essential fatty acids. Unfortunately, if preparing VLCPUFAs by chemical syntheses, these have resulted in only a limited number of VLCPUFAs compared to those being present in important body tissues. Further, it has been common to believe that VLCPUFAs are synthesized in the relevant tissues and do not come from the diet. Hence, relevant compositions comprising a variety of fatty acids including VLCPUFAs have not been commercially available.

Based on the above, there is a need for new and alternative treatment of diseases and conditions of subjects, and particularly of those subjects having a reduced ability for endogenic synthesis of fatty acids.

### BRIEF SUMMARY OF THE INVENTION

It is therefore an object of the present invention to provide methods and compositions which are useful in the treatment and alleviation of diseases, symptoms and conditions associated with a reduced ability for endogenic synthesis of fatty acids, such as of those having deficiencies in one or more elongase system.

The invention further provides a composition comprising VLCFAs for use in treatment of diseases, symptoms and conditions that may be improved by an increased concentration of

VLCFAs in specific tissues. In one embodiment, the subject has a deficient or abnormal level of VLCFAs present in specific tissue which play a role in the disease.

The applicant envisages that deficiencies in one or more elongase system, and/or other enzymatic systems may be alleviated by administration of very long chain fatty acids (VLCFAs) of natural origin.

### BRIEF DESCRIPTION OF THE DRAWINGS

FIGS. 1-8 provide the content of different fatty acids in eye (apple) tissue from mice fed different Test Diets.

FIGS. 9-16 provide the content of different fatty acids (μg/g tissue) identified in blood plasma from mice fed Test Diets 1, 2 and 3.

FIGS. 10-24 provide the content of different fatty acids (mg/g tissue) identified in eye apple tissue from Salmo salar fed 5 different test diets.

FIG. 25 provides the levels of VLCPUFAs identified in brain, eyes and skin tissue of rats fed three different diets; plant oil, fish oil or plant/fish oil.

FIG. 26 shows the identified VLC-PUFAs in the brain, eye and skin phospholipids of Atlantic salmon fed three different levels of two fish oils.

FIG. 27 provides a fluorescence image of ATCC human fibroblasts supplemented with 4 μM Lipid composition A in culture media, wherein a scratch was created, and the migration of cells into the scratch/closure of wound was followed over time for different concentrations of the lipid composition A.

FIG. 28 provides the measurement of cell proliferation of a dermal fibroblast cell line after incubation with Lipid composition B until about 50% confluency.

FIG. 29 provides the effect of a Lipid composition B on closure rate of a scratch wherein Human ATCC dermal fibroblasts were incubated with Lipid composition B, cells were scratched and cell migration was followed at different time points.

FIG. 30 shows the cell migration from salmon shells, wherein shells were placed in wells with culture medium, treated with Lipid composition B at two different concentrations and inspected for cell migration the following days.

FIGS. 31-33 show the content of some major VLC fatty acids in skin tissue in mouse fed different diets.

FIGS. 34 to 37 show the content of the major VLC fatty acids in brain tissue in mouse fed different diets.

FIGS. 38-41 show the content of the major VLC fatty acids in testis tissue in mouse fed different diets.

FIGS. 42-43 show the content of some major VLC fatty acids in the PL fraction of liver tissue in mouse fed different diets.

FIGS. 44-46 show the content of some major VLC fatty acids in the TAG fraction of liver tissue in mouse fed different diets.

FIGS. 47-48 show the content of some major VLC fatty acids in the PL-fraction of heart tissue in mouse feed different diets.

FIGS. 49-51 show the content of some major VLC fatty acids in the TAG fraction of heart tissue in mouse feed different diets.

FIGS. 52-54 show the content of some major VLC fatty acids in skin tissue in salmon fed different diets.

FIGS. 55-56 show the content of some major VLC fatty acids in brain tissue in salmon fed different diets.

FIGS. 57-59 show the content of some major VLC fatty acids in PL fraction of liver tissue in salmon fed different diets.

FIGS. 60-62 show the content of some major VLC fatty acids in TAG fraction of liver tissue in salmon fed different diets.

FIGS. 63-65 show the content of some major VLC fatty acids in PL fraction of heart tissue in salmon fed the different diets.

FIGS. 66-68 show the content of some major VLC fatty acids in TAG fraction of heart tissue in salmon fed different diets.

FIG. 69 shows the microanatomy of skin from Atlantic salmon showing the different layers.

FIG. 70 shows measures of salmon skin microanatomy including counting of mucous cells, thickness of the epidermis and dermis, as well as evaluation of scale development.

FIG. 71 shows the development of epidermis thickness in fish over time showing more mature scales over time.

FIG. 72 shows the measured epidermal thickness of fish fed feed comprising different concentrations of VLCPUFAs.

FIGS. 73-74 show the content of two VLCMUFAs in skin tissue from mice fed three different test diets.

FIGS. 75-76 show the content of two VLCMUFAs in the neutral lipid fraction of skin tissue from mice fed different test diets.

FIG. 77 shows the content of C24:1 in blood plasma from mice fed different test diets.

### DETAILED DESCRIPTION OF THE INVENTION

Hence, some subjects may experience reduced ability for endogenic synthesis of fatty acids, as they have a reduced ability to synthesize longer fatty acids from shorter fatty acids. Thus, these subjects may have a reduced ability for endogenic synthesis of longer fatty acids, such as fatty acids with a chain length above C22, from fatty acids of a shorter length. Such reduced ability for endogenic synthesis may be in specific tissues where these fatty acids are needed for maintaining the subjects' optimal health. The reduced ability may develop with age or may be present already at young age. Especially in the latter case, reduced ability for endogenic synthesis of very long chain fatty acids may be caused by hereditary diseases. Hence, the diseases associated with the deficient endogenic synthesis may be familial or acquired.

Compositions containing concentrates of C20-C22 omega-3 fatty acids are often recommended in order to treat or alleviate symptoms of different conditions, and such fatty acids are included in both pharmaceuticals and supplements. However, not all subjects respond satisfactory to treatment e.g. with high concentrations of EPA and DHA. A reason may be that the subject's body is not able to metabolize or use the administered fatty acids sufficiently, e.g. to produce longer fatty acids in vivo. A deficient elongase system, or other enzymatic system, may be the reason for the reduced response to traditional C20-C22 omega-3 fatty acid treatment. Hence, the applicant has realised that in some instances it is actually the presence of very long chain fatty acids (VLCFAs), i.e. having a chain length of at least C24, that provide the beneficial effect. Hence, it is the VLCFAs normally produced in vivo from administered long chain fatty acids that provide the beneficial effect, and subjects that have an enzymatic system, such as an elongase system, with reduced effect will not be able to produce the beneficial VLCFAs from the administered fatty acids in optimal amounts.

Hence, biologically beneficial PUFAs, including omega-3 fatty acids, are not limited to the long chain fatty acids such as EPA, DHA and n3DPA. As disclosed by Breivik and Svensen in WO2016/182452 there is only small amounts of the VLCn3s in natural oils like fish oils, and these and other very long chain fatty acids are normally substantially removed during production of traditional marine omega-3 concentrates, where the aim is to up-concentrate omega-3-fatty acids with chain length C20-C22. Hence, in conventional omega-3 fatty acid supplements, any very long chain fatty acids have been substantially removed, and such supplements are not suitable for obtaining VLC omega-3 fatty acids.

Unfortunately, however, when isolating VLCFAs from natural oils, like marine oils, e.g. oils of organisms like fish, crustaceans etc, algal oils, or oils of higher plants, the fatty acid chain lengths of the VLCFAs are often shorter than those of many of the VLCPUFAs which are known to exhibit positive biological effects in tissues related to e.g. the healthy eye, male fertility, skin, epidermal and mucosal tissues (including lungs and respiratory tract), brain and nervous systems. Nevertheless, the VLCFAs of natural oils have now been found beneficial in treatment of various diseases associated with these tissues, and have a surprisingly good effect as explained below. VLCPUFAs are for example found in tissues associated with high expression of for example ELOVL4.

The applicant has realised that deficiencies in one or more elongase system, or other enzyme systems as discussed below, may be alleviated by administration of very long chain fatty acids (VLCFAs) from natural oils, like marine oils. Without wishing to limit ourselves to specific explanations: As discussed in further detail below, the various elongase enzyme systems (inter alia also desaturase and β-oxidation reactions) of the body are involved in the in vivo syntheses of numerous fatty acids. This includes the fatty acids with chain length up to C22, and also VLCn3s, VLCn6s, VLCMUFAs, like for example n9 MUFAs, and VLCSFAs, leading to competition among fatty acids for these enzymes. If in a subject one or more of these in vivo systems exhibit a reduced efficiency, compared to a subject with normal exhibiting systems, one or more “bottlenecks” for the in vivo synthesis of VLCFAs could be created.

As a simplified example we could think of the fatty acid C22:5n3 (n3DPA) acting as an intermediate for in vivo synthesis of C24:5n3 via an elongase system with reduced efficiency. The reduced efficiency of the elongase system, and the competition with numerous other fatty acids for the same system, would create a “bottleneck”, leading to a reduced synthesis of C24:5n3 compared to that of a subject having a normally efficient elongase system. In order to obtain a VLCPUFA of the schematic structure C(24+2x):5n3, the diminished concentration (compared to normal) of C24:5n3 would have to compete for x new passages through the “bottleneck” elongase system. Due to competition with other fatty acids requiring this same elongase system, which exhibits a reduced efficiency, for each passage the relative concentration of successive intermediates of the VLC fatty acid C(24+2x):5n3 would be further reduced, compared to a subject with an optimal elongase system. If, for purpose of illustration only, we assume that the elongase systems have a reduced efficiency of 50% compared to optimal for each of the (x+1) steps from C22:5n3 (n3DPA) via C24:5n3 to the VLCPUFA of schematic structure C(24+2x):5n3, an elongated fatty acid of the structure C32:5n3 (x=4; x+1 =5) would be produced in vivo at a rate of (0.5)⁵, i.e. 3%, compared to the optimal rate. Similarly, if the efficiency was reduced to 80%, in the illustrative calculation C32:5n3 would be produced in vivo at a rate of (0.8)⁵, i.e. 33% compared to optimal rate, while if the efficiency was reduced to 20%, in the illustrative calculation C32:5n3 would be produced in vivo at a relative rate of (0.2)⁵ compared to the optimal, i.e. just 0.03% of the optimal. However, if, for illustration, the body was supplemented with an adequate amount of C28:5n3, so that C28:5n3, could act as starting material for in vivo synthesis of C32:5n3 (x =2) the similar relative rates compared to optimal would be (0.5)², i.e. 25%; (0.8)², i.e. 64%; and (0.2)², i.e. 4%, respectively.

The calculation above illustrates that VLCPUFA compositions, e.g. as disclosed herein, can highly improve the body's in vivo synthesis of biologically active VLCPUFAs compared to traditional long chain omega-3 concentrates from marine oils.

If a subject is consuming little or no marine omega-3 fatty acids, such as from the food, the main omega-3 fatty acid in the diet could be expected to be C18:3n3 (ALA), adding a further two in vivo elongation steps needed to obtain a fatty acid of the structure C(24+2x):5n3. In addition, two desaturase steps would be required to reach the 5 double bonds in C(24+2x):5n3, compared to 3 in C18:3n3. Hence, if a subject does not consume marine omega-3 fatty acids, either e.g. because of allergy, diet issues, or preferences, LCPUFAs will in a less degree be available in relevant tissues for further elongation. The subject may hence be deficient of LCPUFAs. Such subject may benefit from a supplementation of fatty acids from compositions as disclosed, comprising VLCFAs, also even if the subject has a normal ability for endogenic synthesis of VLCFAs. Hence, the invention provides a composition comprising VLCFAs for use in treatment of a subject's disease or condition that may be improved by an increased concentration of VLCFAs in specific tissues. When administering the composition of VLCFAs the fatty acids are taken up by target body tissues, where VLCFAs play a role for a normal tissue function. Hence, the composition is for use in prevention or treatment of a disease by administering VLCFAs, which are transported to target body tissues where these play a role for a normal tissue function.

By administering compositions of VLCFAs according to the present application to a subject, “bottlenecks” similar to those described above can be overcome. Particularly, for whom one or more of the in vivo systems for synthesis of fatty acids exhibit a reduced efficiency, such “bottlenecks” can be overcome. Even for situations where the VLCFAs that are administered according to the present application have shorter chain lengths, and/or contain a different number of double bonds, than those of the VLCPUFAs which give desired positive health effects, a surprisingly high degree of alleviation of the patient's health may be obtained.

VLCPUFAs are normally found in specific body tissues, including in tissues of: the eyes (eyeball, retinas, meibum from the meibomian glands in the eyelids), sperm and testes, brain and nervous systems, the various epidermal and mucosal tissues/mucous membranes, including lung and respiratory tract. Sebaceous glands are microscopic exocrine glands in the skin that secrete an oily or waxy matter, called sebum, to lubricate and waterproof the skin and hair of mammals. In humans, they occur in the greatest number on the face and scalp, but also on all parts of the skin except the palms of the hands. In the eyelids, meibomian glands, are a type of sebaceous gland that secrete a special type of sebum into tears. There is increasing evidence that sebaceous fatty acids play a role in the maintenance of skin barrier integrity. As understood in the present application, a mucous membrane or mucosa is a membrane that lines various cavities in the body and covers the surface of internal organs. It consists of one or more layers of epithelial cells overlying a layer of loose connective tissue. It is mostly of endodermal origin and is continuous with the skin at various body openings such as the eyes, ears, inside the nose, inside the mouth, lip, vagina, the urethral opening and the anus. Some mucous membranes secrete mucus, a thick protective fluid. The function of the membrane is to stop pathogens and dirt from entering the body and to prevent bodily tissues from becoming dehydrated. Hence, VLCFAs are normally present in various tissue and have a function there. Future research will probably result in more knowledge regarding in vivo synthesis of VLCFAs, in which tissues such syntheses take place, and which tissues and body functions that benefit from VLCFAs.

As documented by the Examples below, VLCPUFAs and VLCMUFAs administered to a subject are taken up by specific body tissues of the subject, to provide a positive health effect. More particularly, the administered VLCFAs are transported to specific tissue and taken up in such tissue which normally have VLCFAs present, and wherein this specific tissue has a role in the disease or in a condition. Hence, the invention provides a composition comprising VLCFAs for use in treatment of diseases that may be improved by an increased concentration of VLCFAs in specific tissue. The specific tissue wherein uptake takes place is e.g. the eyes (eyeball, retinas, meibum from the meibomian glands in the eyelids), sperm and testes, brain and nervous systems, the various epidermal and mucosal tissues/mucosal membranes, including lung and respiratory tract, tissue of the cardiovascular system, and of the urine bladder, urinary system, and the digestive system.

Surprisingly, deficiencies in one or more enzymatic systems, such as an elongase system, may be alleviated by administration of very long chain fatty acids (VLCFAs). And further, administered VLCFAs are taken up by relevant tissue. By the term VLCFA, VLCPUFAs, and also VLCn3, VLCMUFA, VLCSA, and VLCn6 are included. And, as is employed herein, the term very long chain fatty acids (or VLCFAs) is intended to mean fatty acids (FAs) having a chain length of more than 22 carbon atoms, i.e. having at least a C24 chain length; the term very long chain polyunsaturated fatty acids (VLCPUFAs) is intended to mean polyunsaturated fatty acids (PUFAs) having a chain length of more than 22 carbon atoms; the term very long chain monounsaturated fatty acids (VLCMUFAs) is intended to mean monounsaturated fatty acids (MUFAs) having a chain length of more than 22 carbon atoms; while the term VLCn3 is intended to refer to polyunsaturated omega-3 fatty acids having a chain length of more than 22 carbon atoms, it being understood that VLCn3 represents a sub-group of VLCPUFA. Similarly, the term VLCn6 is intended to refer to polyunsaturated omega-6 fatty acids having a chain length of more than 22 carbon atoms. Very long chain saturated fatty acids (VLCSA) is intended to mean saturated fatty acids having a chain length of more than 22 carbon atoms. Hence, VLCFAs used herein have a chain length of C24-C40, such as C24-C38, and preferably of C24-32. VLCFAs used herein have a chain length of C24-C40, such as C24-C38, and preferably of C24-32. In some embodiments, VLCFAs used herein have a chain length of C26-C40, such as C26-C38, and preferably of C26-32. In some embodiments, VLCFAs used herein have more than 6 double bonds, preferably 7 or 8 double bonds, and even more preferred being VLCn3 fatty acids with length of C28-C32 having 7 or 8 double bonds.

Very long chain fatty acids confer functional diversity on cells by variations in their chain length and degree of unsaturation. In vivo fatty acid elongation occurs in three cellular compartments: the cytosol, mitochondria, and endoplasmic reticulum (microsomes). In the cytosol, fatty acid elongation is part of de novo lipogenesis and involves acetyl-CoA carboxylase and fatty acid synthase. Fatty acid synthase utilizes acetyl CoA and malonyl CoA to elongate fatty acids by two carbons. Microsomal fatty acid elongation represents the major pathway for determining the chain length of saturated, monounsaturated, and polyunsaturated fatty acids in cellular lipids. The overall reaction for fatty acid elongation involves an elongase system of four enzymes and utilizes malonyl CoA, NADPH, and fatty acyl CoA as substrates. The pathway involves a family of enzymes involved in the first step of the reaction, i.e., the condensation reaction. Seven fatty acid elongase subtypes (ELOVL #1-7) have been identified in the mouse, rat, and human genomes. These enzymes determine the rate of overall fatty acid elongation. Moreover, these enzymes also display differential substrate specificity, tissue distribution, and regulation, making them important regulators of cellular lipid composition as well as specific cellular functions. Methods to measure elongase activity, analyse elongation products, and alter cellular elongase expression are described by Jump, D., Methods Mol Biol. 2009; 579, 375-389.

In the body, the VLCPUFAs are hence produced in vivo from shorter fatty acids by fatty acid chain elongation, and for certain fatty acids, inter alia, also by desaturation, saturation and β- and ω-oxidation reactions.

From the above, fatty acid elongation takes place in complex reactions that result in two carbons being added to the carbonyl end of fatty acids. From the nomenclature that is used here this means that after elongation an omega-3 acid still remains an omega-3 acid after the elongation, i.e. the fatty acid C20:5n3 (EPA) can be elongated to C22:5n3 (n3DPA), which again can be further elongated to C24:5n3 etc. Similar in vivo reactions take place for omega-6 PUFAs, for other PUFAs, for MUFAs and for SFAs.

In addition to elongation, there is also, inter alia, a need for in vivo desaturation reactions wherein carbon/carbon bonds are created, and for steps for reduction of chain length. For example, the above mentioned C24:5n3 can pass through a Δ6 desaturase reaction to form C24:6n3, creating a double bond, which then through a β-oxidation reaction, with the effect of removing two carbons, can result in C22:6n3 (DHA). Hence, in the biosynthesis of essential fatty acids, an elongase may alternate with different desaturases (for example, Δ6desaturase) repeatedly inserting an ethyl group, then forming a double bond.

The inventors of the present invention have realised that by utilising compositions according to the invention which further comprise DHA (C22:6n3), a subject's endogenic synthesis of VLCPUFAs can be enhanced, as the endogenic synthesis system's need to synthesise C22:6n3 from C24:6n3 is reduced or fully eliminated. Thus, C24:6n3, and/or its biological precursor C24:5n3, to a greater extent can be utilised for endogenic synthesis of more long-chain VLCn3s. This means that VLCPUFA compositions according to the invention can exhibit a surprisingly increased effect by the presence of DHA. In some embodiments, VLCFA compositions may beneficially comprise n3DPA (C22:5 n3), for example in order to reduce or eliminate the endogenic synthesis system's need to synthesise 22:5n3, and/or to improve the endogenic synthesis system's ability to synthesise 24:5n3 from 22:5n3.

Various elongase, desaturase and β-oxidation reactions, like those discussed above for DHA, are also involved in the in vivo syntheses of other fatty acids, including VLCn3s, VLCn6s, VLCMUFAs, like for example n9 MUFAs, and VLCSFAs, leading to competition among fatty acids for these enzymes.

As stated above, for mammals at present seven elongation systems/elongases for VLCPUFAs (ELOVL1-7) have been identified, with each elongase exhibiting a characteristic substrate specificity and tissue distribution. This means that a deficiency in one particular elongase system will have negative biological effects that normally cannot be compensated by the other elongase systems.

For example, an illness like diabetes affects the expression level of elongases and desaturases. This effect on elongases is very strong on ELOVL4, an elongase that can elongate VLCPUFAs, VLCMUFAs and VLCFAs.

ELOVL4 is also expressed in the thymus, i.e. in lymphatic tissue, and there are indications that this has a role in the immune system and preparation of signal molecules.

ELOVL4 is the highest expressed elongase in the retina, and produces VLCPUFA and VLCSA, which are important for the healthy eye. Malfunction of ELOVL4 can be caused by aging, leading to onset of age-related macular dystrophy AMD, by hereditary diseases like the one that is associated with Stargardt-like macular dystrophy (STGD3), and by metabolic diseases like diabetes, which can result in reduced vision and of inflammation of the retina.

ELOVL4 also has an important role in the skin, producing VLCSAs which are incorporated into ceramides which are essential in maintaining the water barrier in skin. The stratum corneum is the outermost layer of the epidermis, consisting of dead cells (corneocytes). These corneocytes are embedded in a lipid matrix composed of ceramides, cholesterol, and free fatty acids. The stratum corneum functions to form a barrier to protect underlying tissues from infection, dehydration, chemicals and mechanical stress. During the process whereby living keratinocytes are transformed into non-living corneocytes, the cell membrane is replaced by a layer of ceramides which become covalently linked to an envelope of structural proteins. This complex gives an important contribution to the skin's barrier function, and is also considered having an important function in keeping the skin appearing healthy, avoiding wrinkled skin and also protecting against negative effects on the skin from the sun's UV radiation.

Endogenic biological systems may be utilised to transfer VLCFAs into w-hydroxy fatty acids, including (O-acyl) ω-hydroxy FAs (OAHFAs). ELOVL4 appears to be involved in the synthesis of VLC ω-hydroxy fatty acids. Wenmei et al. (Wenmei L, Sandhoff R, Kono M, Zerfas P, Hoffmann V, Ding B C-H, Proia R L and Deng C X, Depletion of ceramides with very long chain fatty acids causes defective skin permeability barrier function, and neonatal lethality in ELOVL4 deficient mice, Int. J. Biol. Sci. 2007 3(2):120-128) found that ceramides containing ω-hydroxy very long chain fatty acids (C≥28) are essential components of the epidermal permeability barrier, and that there is an indispensable role for ELOVL4 in the formation of the very long chain fatty acids that serve as constituents of sphingolipids in the epidermal barrier. According to Wenmei et al, in ELOVL4 deficient mice, ceramides with fatty acids ≥C28 were absent or substantially reduced compared to controls. The majority of epidermal VLCFAs with more than 26 carbon atoms in length is ω-hydroxylated and may be saturated or unsaturated (1-2 double bonds). Shingolipids with these fatty acids are ceramides and glucosylceramides (Sandhoff (2010) Very long chain sphingolipids: Tissue expression, function and synthesis, FEBS Letters 584 1907-1923, see section 1.2, first paragraph). These molecules form important parts of the protective function of the epidermis.

Endogenic biological systems other than the elongase systems may also be utilised to transfer LCFAs, including VLCMUFAs and VLCFAs, into the beneficial (O-acyl)-ω-hydroxy FAs (OAHFAs), cholesteryl esters, ceramides, free fatty acids, phospholipids, sphingomyelins and wax esters. The composition according to the invention comprising VLCFAs, although on another form than ω-hydroxy fatty acids, can be used to provide these very important fatty acids to the relevant tissue, especially to the skin and to the mucous membranes/tissues. This can be particularly important for compositions according to the invention comprising VLCFAs with chain length of C28 and above.

Of note, Wenmei et al. found that the ELOVL4 deficient mice showed no desire to find the nipple and suck milk of their mothers. The authors suspected that this reflected a neurological behaviour abnormality due to the absence of ELOVL4 in the brain. The composition according to the invention could represent a way to alleviate such neurological behaviour by providing VLCFAs to the brain.

The following discussion of ELOV1-3 and 5-7 is to a large extent based on Sassa and Kihara (2014) Metabolism of very long chain fatty acids: Genes and pathophysiology, Biomol Ther 22(2): 83-92. However, future research will probably result in more knowledge regarding in vivo synthesis of VLCFAs, in which tissues such syntheses take place, and which tissues and body functions that benefit from VLCFAs.

ELOVL1 elongates saturated and monounsaturated C20-C26 acyl-CoAs. ELOVL2 elongates C20-C22 polyunsaturated acyl-CoAs of both the n3 and n6 series. ELOVL2 deficiency can cause reduction of VLCPUFAs, including C28:5n6 and C30:5n6, in the testis, with reduction of spermatogenesis and male fertility. Mammalian testis and spermatozoa contain both n3 and n6 VLCPUFAs.

ELOVL3 and ELOVL7 are known to elongate both saturated and unsaturated C16-C22 acyl-CoAs.

ELOVL3 is known to be expressed in skin sebaceous glands and hair follicles, and in brown adipose tissue. From research in mice it is shown that deficiency in ELOVL3 exhibit accumulation of C20:1 in the skin, and being associated with defects in water repulsion and sparse hair coat. By reducing inflammation of hair follicles, and by other at present unknown mechanisms VLCFAs may prevent hair loss and improve overall hair health. Mice with deletion of ELOVL3 do not suffer from rapid neonatal death due to water loss in the same manner as ELOVL4 (Sandhoff 2010), showing that the ELOVL3 elongase system leads to different effects than those of ELOVL4.

ELOVLS is considered to be essential for the elongation of C18-CoAs of both n3 and n6 series in the liver. Deletion of ELOVLS in mice is associated with hepatic steatosis. It is highly expressed in the adrenal gland and testis, and encodes a multi-pass membrane protein that is localized in the endoplasmic reticulum. Mutations in this gene have been associated with spinocerebellar ataxia-38 (SCA38), a rare form of ataxia.

ELOVL6 elongates shorter fatty acids compared to other ELOVs, with activity toward C12:0-16:0 acyl-CoAs. Cytoplasmic expression in several tissues, including in the liver, has been shown.

Reduced effect of one or more of pathways similar to those above, can create bottlenecks in the system for in vivo elongation, and subsequent beta oxidation and desaturation, reactions to form VLCFAs that are essential for optimal health. As presented above, these bottlenecks can take place in more than one place in the complicated in vivo syntheses, where at the present date probably not all details have been elucidated.

Hence, individual subjects may experience reduced ability for endogenic synthesis of VLCFAs, including VLCMUFAs and VLCPUFAs in specific tissues where these fatty acids are needed for maintaining the subjects' optimal health. This reduced ability may develop with age, or may be present already at young age. Especially in the latter case, reduced ability for endogenic synthesis of VLCFAs may be caused by hereditary diseases.

Infants require DHA for developing tissues, but do not have fully developed enzymatic systems. The applicant has realised that for optimal health, infants, and particularly those who do not receive mother's milk, will benefit from supplementation of VLCPUFAs of natural origin, in addition to the well-recognised supplementation of DHA (e.g. including infant formula, medicinal food for infants).

It has now been realised that VLCPUFAs from natural oils, like those described herein, administered to a subject can be absorbed in the subject's body, and that deficiencies in one or more elongase system and/or desaturase and/or β-oxidation system may be alleviated by administration of VLCFAs (including VLCn3, VLCn6, VLCMUFA, VLCSA) with chain length C24-C40, such as C24-C32. As further discussed below, and as shown in the Examples, supplemented VLCFAs are taken up by different body tissues, were they can perform their function.

According to the present invention it is further realised that the various groups of VLCFAs as described above, and in more detail below, in certain embodiments can be given together, while in certain other embodiments one or more sub-groups of VLCFAs, i.e. one or more of VLCn3, VLCn6, VLCMUFA, VLCSA, e.g. with chain length C24-C32, can be enriched compared to the other(s) in order to increase the effect of the VLCFA compositions. As VLCFAs confer functional diversity on cells by variations in their chain length and degree of unsaturation, the administered composition may in one embodiment comprise a mixture of several different fatty acids, of various lengths and degree of unsaturation, as disclosed below. By using such VCLFA-enriched compositions, a competition among fatty acids for the enzymes involved in the desired elongase, desaturase and β-oxidation reactions is avoided, and thus the desired group of VLCFA “building blocks” will be channeled through to the final VLCFAs. The term VLCFAs as used here is to be understood to include further in vivo transformations of the VLCFAs. As an example, the term includes hydroxy-derivatives of VLCFAs as formed in vivo, including ω-hydroxy VLCFAs, and further in vivo transformations of the ω-hydroxy VLCFAs.

In the body, the final VLCFAs as described above may for their beneficial actions be present in numerous forms, including, but no way limited to, (O-acyl)-ω-hydroxy FAs, cholesteryl esters, ceramides, free fatty acids, glycerides, phospholipids, sphingomyelins and wax esters.

A subject having deficiencies in one or more of the complex systems for endogenic synthesis may not be able to, or may only in a lower degree than normally, produce VLCFAs from short and long chain fatty acids. Deficiencies in the enzymatic systems may include mutations and small deletions in the ELOVL genes, and such may be linked to disease. Conditions and diseases that may be improved by an increased concentration of VLCFAs, normally produced by fatty acid elongation in vivo, may be worsened if such deficiencies exist. The subject may hence suffer from a reduced ability for endogenic synthesis of VLFAs, i.e. such as caused by a low concentration of any of the enzymes involved in the synthesis, resulting in a lower and/or slower degree of synthesis of fatty acids.

In one aspect, the invention provides a method of treating a subject, by administering to the subject a composition comprising VLCFAs. The VLCFAs have chain lengths of C24-C40, such as C24-38, or such as C24-C32. Equally, the invention provides a composition comprising VLCFAs for use in treatment of a subject. Relevant diseases that can be treated and relevant compositions are disclosed herein. In one embodiment, the disease is associated with a deficiency in one or more endogenous systems and/or with a reduced ability for endogenic synthesis of VLCFAs. In one embodiment, the subject has a deficient or abnormal level of VLCFAs present in specific tissue which play a role in the disease. The examples show that administered VLCFAs are taken up by different tissues. Further, positive effects of the administered VLCFAs have been shown, such as on skin. This new knowledge is combined with the knowledge that VLCFAs are normally present in different tissues, and with that of disease-promoting reductions in enzyme activity. Please see discussion below about intrinsic and extrinsic factors which may affect patterns of aging, and which also is relevant for other diseases and conditions.

In one embodiment, the invention provides a composition comprising at least 5% by weight of VLCFAs for use in treatment of a subject, wherein the composition is administered to the subject for treatment, the subject has a deficient or abnormal level of VLCFAs present in specific tissue which play a role in the disease.

In one embodiment, the invention provides a composition comprising at least 5% by weight of VLCFAs for use in treatment of a subject, wherein the composition is administered to the subject for treatment related to a deficiency in one or more endogenous elongase systems and/or with a reduced ability for endogenic synthesis of VLCFAs.

In one embodiment, the invention provides a composition comprising at least 5% by weight of VLCFAs for use in treatment of a disease of a subject, wherein the composition is administered to the subject. In one embodiment, the disease is associated with a deficiency in one or more endogenous elongase systems and/or with a reduced ability for endogenic synthesis of VLCFAs.

Hence, in one embodiment, the invention provides a composition comprising at least 5% by weight of VLCFAs, having a chain length of more than 22 carbon atoms, and isolated from natural oils, for use in treatment of a subject, wherein the composition is administered to the subject for treatment related to a deficiency in one or more endogenous elongase systems and/or with a reduced ability for endogenic synthesis of VLCFAs, or for prevention or treatment of a disease, wherein the administered VLCFAs are transported to target body tissues where these play a role for a normal tissue function.

As used herein, the term “disease” refers to either of diseases, conditions, disorders or ailments. Particularly, the method of the invention and the composition for use of the invention are useful in treatment of diseases which are associated with or involve particular tissues which normally comprise VLCFAs. Relevant tissues are selected from the following non-limited group of, e.g. tissue of the eye (eyeball, the retinas or meibum), sperm and testes, brain and nervous systems, skin, epidermal and mucosal membranes/tissues, including tissues of the lung and respiratory tract, tissue of the cardiovascular system, and of the urine bladder, urinary system and digestive system.

Particularly, the treatment may be for maintaining normal tissue function by supplying the tissues with VLCFAs, wherein the administered VLCFAs can help maintain good functions in tissues known to normally have the VLCFAs present. For example, the addition of the VLCFAs to the different tissues can contribute to a direct, amended or improved fluidity of cell membranes. Such treatment, including treatment of diseases, by administering the composition for use, include, or are related to, either of eye health, male fertility, diseases of the skin and/or endothelial and mucosal tissues/mucous membrane, brain and nervous tissue, and cardiovascular diseases. Diseases of the skin and/or endothelial and mucosal tissues/mucous membrane are for example diseases of the urine system and digestive system, and also includes eczema, allergy and lung diseases such as asthma.

By the cardiovascular system, we mean to include the organ system that conveys blood through vessels to and from all parts of the body, including the pulmonary and the systemic circuits, consisting of arterial, capillary, and venous components. Hence, tissue of the blood vessels and the cardiac muscle tissue are included, and diseases related to these. Any of the cardiovascular diseases, whether congenital or acquired, of the heart and blood vessels, are relevant for treatment by the composition for use of the invention. Among the most important are atherosclerosis, rheumatic heart disease, and vascular inflammation.

The compositions for use, may be used in treatment of eye diseases that are negatively affected by reduced amounts of VLCFAs. These include age-related macular degeneration (AMD), diseases caused by diabetic inflammation of the eye, and dominant Stargardt macular dystrophy (STGD3). These are typically caused by mutations in the ELOVL4 gene. For the latter reason STGD3 usually occurs in childhood or adolescence. Dry eye disease (DED) and meibomianitis are diseases related to the eye.

In AMD there is a progressive accumulation of characteristic yellow deposits, called drusen (buildup of extracellular proteins and lipids), in the macula. Studies indicate drusen associated with AMD are similar in molecular composition to β-amyloid (βA) plaques and deposits in other age-related diseases such as Alzheimer's disease and atherosclerosis. This suggests that similar pathways may be involved in the etiologies of AMD and other age-related diseases.

Diseases related to the brain and nervous tissue, including diseases of the central nervous system, that may be treated by the compositions of the invention, comprise at least the following; Reduced mental health, demyelinating diseases such as multiple sclerosis, Parkinson's, Schizophrenia, Dementia, Alzheimer's, impaired cognitive function, migraine, seizures and epilepsy.

For the treatment of male fertility, the use of the VLCFA composition may enhance the function and/or viability of the sperm, or to increase the amount of mature sperm cells.

Diseases related to the skin and hair that may be treated by the composition for use of the invention, comprise at least the following: dry and wrinkled skin, irritated, sour or sensitive skin, ability for wound healing, as protection (i.e. preventive treatment) against negative effects on the skin from the sun's UV radiation, negative effects on hair follicles, reduced hair health including risk of hair loss. Examples of skin diseases and conditions that typically give irritated/sour skin and which may benefit from treatment with the compositions for use are e.g. eczema, psoriasis, acne and rosacea (papulopustular rosacea). By use of the composition or method of the invention one can normalize the fatty acid composition of a tissue, such as of the skin, such as by compensating for an abnormal sebaceous fatty acid composition, i.e. compensating for a reduced level of endogenic synthesized very long chain fatty acids.

It is known that infants require DHA for developing tissues, but do not have fully developed enzymatic systems. DHA is an important fatty acid component in human milk, and for this reason it is normal to add DHA to infant formula, and for medicinal nutrition to infants. The applicant of the present invention has realised that for optimal health infants will also benefit from supplementation with VLCPUFAs of natural origin. In one embodiment, the present invention provides a composition for addition to nutrition to infants, such as baby food, infant formula and medicinal nutrition, the latter including nutrition given parenterally. According to the present invention, an infant refers to infants in utero and to children less than about two years of age, including premature and new-born infants. Hence, the composition may also be administered to pregnant women as part of supplemental nutrition, for contribution to the development of the fetus, and may be administered as an oral or parenteral formulation.

Also diseases associated with a reduced immune system may be treated by the composition for use. Particularly, the fatty acids contribute to strengthen the skin, epidermal and mucosal tissues/mucosal membranes forming a barrier to protect underlying tissues from pathogens, including infections, inflammations, dehydration, chemicals and mechanical stress. Administered VLCFAs have also now been shown to be taken up by immune cells, please see example section, wherein Example 1 shows that VLCPUFAs included in mice's diet are taken up by blood plasma.

Further, inflammatory related diseases and cardiovascular diseases may be treated by the composition for use, particularly e.g. atherosclerosis and rheumatoid arthritis.

As used herein the term “treating” or “treatment” refers to 1) inhibiting the disease; for example, inhibiting a disease, condition or disorder in a subject who is experiencing or displaying the pathology or symptomatology of the disease, condition or disorder, including prevention of disease (i.e. prophylactic treatment, arresting further development of the pathology and/or symptomatology), or 2) alleviating the symptoms of the disease, or 3) ameliorating the disease; for example, ameliorating a disease, condition or disorder in an subject who is experiencing or displaying the pathology or symptomatology of the disease, condition or disorder (i.e., reversing the pathology and/or symptomatology). Particularly, in one embodiment the composition for use is for preventive treatment, such as for maintaining normal tissue function, or improving tissue function, by supplying the tissues with VLCFAs. The administered VLCFAs can help maintain good functions in tissues known to normally have the VLCFAs present.

As used herein, when referring to a subject, this term encompasses both human and non-human animal bodies, and non-human animals also include fish, such as farmed fish.

Particularly, the invention provides a method for treatment, and a composition for use in treatment, of diseases related to one or more of eye health, male fertility, skin and endothelial tissues and mucosal tissues/mucous membranes, brain and nervous tissue, and cardiovascular tissues, by administration of a lipid composition comprising very long chain fatty acids.

In one embodiment, the invention provides a composition for use in treatment of a subject having deficiencies in one or more endogenous elongase and/or other enzymatic systems necessary for in vivo synthesis of VLCPUFAs. The elongase system and/or other enzymatic system may be important for the health of the subject. The method comprises the step of administering to the subject a lipid composition comprising VLCFAs. The VLCFAs may have a direct positive health effect for the subject, or they may function as “building blocks” for even longer fatty acids that have a direct positive health effect. Accordingly, the VLCFA-containing lipid composition is particularly for use in treatment of a subject group with a reduced ability for endogenic synthesis of VLCFAs. Further, the VLCFAs might as well act as a trigger for expression of enzymes for elongase or desaturase of fatty acids through a kind of epigenetic effect.

Particularly, as mentioned above, ELOVL2 deficiency can cause reduction of VLCPUFAs, including the specific fatty acids C28:5n6 and 030:5n6, in the testis, with reduction of spermatogenesis and male fertility as a result. In one embodiment, the invention provides a composition for use in treatment of a subject's ability for production of healthy sperm, by administering to the subject a composition comprising VLCFAs with a chain length of 24-C32, such as of a chain length of C28-030. More particularly, the composition is enriched with one or more of the fatty acids C28:5n6 and 030:5n6. In one embodiment, the composition is enriched with one or more of the fatty acids 028:5n3, 028:6n3, C28:7n3 C28:8n3 and 030:5n3. Reference is made to Example 1A below, showing that for mice having been fed a diet comprising VLCPUFAs (Test Diet 2), VLCPUFAs from the diet are taken up in the phospholipid fraction of testis tissue.

### Elongases

In one embodiment, the composition for use is for treatment of one or more diseases associated with a deficiency in either of the elongase systems ELOVL 1-7. Non-limiting examples of diseases associated with these enzymes are provided above.

Particularly, in one embodiment, the treatment is directed towards deficiencies in the ELOVL4 enzyme system, and the composition can be used in treatment of diseases associated with such deficiencies, e.g. diseases of the eye, skin or of diabetes. In another embodiment, the treatment is directed towards deficiencies in the ELOVL2 enzyme system, and the composition can be used in treatment of diseases associated with such deficiencies, e.g. in improvement of male fertility. In another embodiment, the treatment is directed towards deficiencies in the ELOVL3 enzyme system, and the composition can be used in treatment of diseases associated with such deficiencies, e.g. in diseases of the skin, hair and of brown adipose tissue. Particularly, it has been found that the compositions improve wound healing as the VLCFAs are taken up by cells of the skin, endothelial tissues or mucosal tissues providing a faster cell division. The wound hence heals quicker. Hence, deficiencies in, inter alia, ELOVL3 or ELOVL 4 resulting in diseases or conditions related to the skin, may be treated according to the invention. When taken up by the skin cells, such as by fibroblasts, the VLCFAs of the composition contribute to strengthening the barrier to protect underlying tissues from infection, dehydration, chemicals and mechanical stress. In one embodiment, the composition for use in treatment of skin further comprises VLCMUFAs, and particularly a-hydroxy VLCMUFA with up to 34 carbons. As found by A. Poulos (1995) Very long chain fatty acids in higher animals—a review, Lipids, 30: 1-14, α-hydroxy VLCMUFAs with up to 34 carbon atoms are found in epidermal lipids. The fatty acid on the α-hydroxy form may be synthesised by modifying the VLCMUFA from natural oil.

In one embodiment, the composition for use is particularly for treatment of farmed fish to strengthen their skin, such as against lice, mechanical stress or for quicker wound healing, and increased survival rate. For instance, the VLCFA composition for use, as disclosed herein, can be included in the feed of the fish. Reference is made to the Examples. Examples 1A and 2B show uptake of VLCPUFAs in skin tissue. Examples 5 and 6 directed to fish fed a VLCPUFA-comprising feed, show positive effect on wound healing, and promoting thicker epidermis and improved scale development.

In another embodiment, the treatment is directed towards deficiencies in the ELOVL5 enzyme system, and the composition can be used in treatment of diseases associated with such deficiencies, e.g. diseases of the liver, such as of hepatic steatosis, or milder forms as fatty liver (non-alcoholic fatty liver, NAFLD). Deficiencies in any of the ELOVL1-7 enzyme systems may be compensated by treatment according to the present invention.

Further, the invention provides a composition for use in improving the concentration of VLCFAs in tissues where such fatty acids are important for the health and well-being of a subject. The applicant has found that the very long chain fatty acids administered to a subject, are absorbed by tissue which normally have such fatty acids present in the tissue. Hence, the applicant has found that a body's insufficiency in synthesising the relevant VLCFAs and for providing the necessary concentration of these in different tissues, can be compensated by administering the relevant VLCFAs to the body, as such VLCFAs actually will be transported to and taken up by the relevant tissue.

In one embodiment, the subject suffers from a reduced effectiveness of one and more of the body's elongase systems. In one embodiment, the composition for use is intended for persons who suffer from age-related reduced effectiveness of one or more of the body's elongase systems. In another embodiment of the invention, the composition for use is intended for persons who suffer from hereditary reduced effectiveness of one or more of the body's elongase systems. Aging is a complex process characterized by a decline in physiological functions and associated with increased risks for various diseases. It is known that methylation of genomes represents a strong and reproducible biomarker of biological aging rate. The methylation pattern enables a quantitative model of the aging, and the model can be used in multiple tissues, acting as a form of common “molecular clock”. As an example, it has been documented that the human elongation gene Elovl2 displays increased methylation with age. The degree of methylation displays high correlation with age, and an almost “on-off” methylation trend between the two extremes of life, ranging from 7% to 91% of methylation in a study that was carried out by Garagnani, P., Bacalini, M. G., et al. (2012) Methylation of ELOVL2 gene as a new epigenetic marker of age. Aging Cell, 11, 1132-1134. https://doi.org/10.1111/ace1.12005. The elongation enzyme ELOVL2 elongates

C20-C22 polyunsaturated acyl-CoAs of both the n3 and n6 series. ELOVL2 is assumed to be present in a number of tissues, including the retina, the liver and in in the testis. Assuming a correlation between increased methylation of the Elovl2 gene and reduced activity of the elongation enzyme, increased age can be considered to correspond with ELOVL2 deficiency, causing reduction of in vivo synthesis of VLCPUFAs. This elongase deficiency caused by age-related downregulation of Elovl2 expression will negatively influence the biological function relating to, inter alia, the healthy eye, male fertility, healthy liver functioning, and neurological functions. Using optimal vision as example: even in healthy human individuals, aging leads to a reduction of visual functions, including age-related decrease in rod-driven, or scotopic, visual acuity and spatial contrast sensitivity. As realised by the inventors of the present invention, the observed age-related loss of rod vison may be related to a decline in physiological functions of elongation genes caused by age, including, but not limited to, the age-related methylation of the elongation gene Elovl2, the latter causing decreased ELOVL2 elongation of C20-C22 polyunsaturated fatty acids. As explained in detail below, the present inventors have realised that the effects of an age decreased ability of elongation enzymes (not limited to ELOVL2) to perform in vivo synthesis of VLCFAs can be ameliorated by supplementation of VLCFAs according to the disclosures of the present invention.

Similarly, for an individual, the effects of age-related decrease in the activity of elongation enzymes in other tissues than the eye, inter alia, in the skin and endothelial tissues, in the testes, neurological tissues and liver, can be ameliorated by supplementation of VLCFAs according to the disclosures of the present invention. Beneficial effects for the individual can include, but are not limited to, improved vision and eye health, improved fertility, improved skin health (including less wrinkling of the skin), improved functioning of brain and neurological tissues, and improved liver functioning.

The inventors envisage that similar beneficial effects as in the case of the various elongases can be obtained to ameliorate age related decrease in the activity of enzymes within all the enzyme groups that are involved in the in vivo synthesis of VLCFAs. As mentioned above, in the body, the VLCFAs are produced in vivo from shorter fatty acids by fatty acid chain elongation. In addition, for certain VLCFAs also other enzyme systems are involved, inter alia, enzyme systems for desaturation, saturation and β- and ω-oxidation reactions.

It is known that DHA deficiency is associated with aging. The applicant is of the opinion that the same is the case for VLCFAs and discloses how compositions comprising VLCFAs as disclosed herein, can alleviate the results of aging effects that are causing these deficiencies.

As referred to above, aging is associated with widespread changes in genome-wide patterns of DNA methylation. Such changes in methylation may be affected both by genetic and environmental factors, in addition to aging itself. Extrinsic environmental factors such as smoking, sun exposure, and obesity, for example, are associated with specific changes in DNA methylation patterns. Intrinsic factors, such as genetic background, can also influence patterns of aging, including “baseline” DNA methylation levels. Treatment methods and compositions according to the present invention are envisaged to ameliorate negative health effects of extrinsic environmental factors and intrinsic hereditary genetic factors to methylation of genomes related to enzymes for in vivo VLCPUFA syntheses and modifications, including, but not limited, to the explicit factors that are referred to above.

In another embodiment the composition for use is intended for infants, e.g. persons who have not fully developed the body's enzymatic systems.

### Eye health

In one embodiment, the invention provides a composition for use for treatment of a subject's disease related to eye health, wherein the subject has deficiencies in one or more endogenous elongase systems that are important for the healthy eye, by introducing to the subject a lipid composition comprising VLCFAs. This may have a direct positive health effect or the VLCFA may function as “building blocks” for even longer fatty acids that have a positive health effect for the healthy eye. The invention hence provides a composition comprising VLCFAs for use in treatment of a subject's eye health wherein an increased concentration of VLCFAs in specific tissue of the eye is obtained. In one embodiment, the disease related to eye health is selected from the group of macular degeneration (AMD), diseases caused by diabetic inflammation of the eye, and dominant Stargardt macular dystrophy (STGD3).

In one embodiment, the invention provides a composition for use for treatment of a subject's disease related to dry eye disease or meibomianitis, wherein the subject has deficiencies in one or more endogenous elongase systems, by introducing to the subject a lipid composition comprising VLCFAs. This may have a direct positive health effect or the VLCFA may function as “building blocks” for even longer fatty acids that have a positive health effect for the DED or meibomianitis. In one embodiment, for the composition for use, wherein the use is treatment of a subject's disease related to eye health, dry eyes disease and meibomianitis are disclaimed. Similar to the paradox derived from the publication of Gorusupudi et al. for AMD, as discussed on page 3, supplementation of omega-3-acids to patients suffering from dry eye disease (DED) has given conflicting results. The inventors of the current invention have looked into the published studies included in the meta-analysis of Giannaccare et al. (2019) on Efficacy of omega-3 fatty acid supplementation for treatment of Dry Eye Disease (DED), to study and possibly identify which types of fatty acids have effects on dry eyes symptoms. The compositions of the omega-3 fatty acid supplementation used in the individual 17 studies of the meta-analysis have been looked into, to identify the presence and concentration of different fatty acids, including the concentration of very long chain fatty acids (VLCFAs). In summary, it appears that studies based on vegetable oils and on marine omega-3 concentrates, where VLCFAs are absent or assumed to be substantially removed in order to concentrate the desired C20-C22 omega-3 acids, tend to show no or limited positive effects on DED, while studies that are based on non-concentrated fish oils and krill oil, and comprising VLCPUFAs, and concentrates of LCPUFA that are containing small amounts of VLCPUFAs (e.g. Study No. 15 of meta-analysis), tend to give clearly positive results for DED patients. This surprising connection between omega-3 fatty acid source and effect, has eluded the authors of the meta-analysis, and also the authors of the individual studies that were included in the meta-study. Giannaccare et al. and the authors of all the individual studies are silent as to the presence or effect of VLCPUFAs/VLCn3s. And it is clear that the positive effect of VLCPUFAs supplementation on DED symptoms were not apparent for the scientific community. As for many other indications, the meta-study on DED focuses on potential effects of C20-C22 omega-3 fatty acids (EPA+DHA). Having studied the meta-analysis and the compositions used, the applicant concludes that it is the VLCFAs of the compositions that contribute to the treating effect, and that a more efficient benefit for alleviating symptoms and treatment of DED can be obtained by administration of compositions comprising VLCFAs, including VLCn3s. Similar effects are expected for other indications of the eyes. As shown in the attached Examples 1, 2 and 3 below, VLCFAs included in diet are taken up by eye tissue. Thus, supplemented VLCFAs that are beneficial for the eye health can reach eye tissue, and there perform their functions. This means that supplementation with compositions of VLCFAs according to the present invention can be utilised in treatment of eye diseases, also other diseases and conditions than DED, such as macular degeneration (AMD), diseases caused by diabetic inflammation of the eye, and dominant Stargardt macular dystrophy (STGD3).

### Male Fertility

In another embodiment, the invention provides a composition for use for treatment of a subject's ability for production of healthy sperm, wherein the subject has deficiencies in one or more endogenous elongase systems that are important for a male person's ability for production of healthy sperm, by introducing to the subject a lipid composition comprising VLCFAs. This may have a direct positive health effect or the VLCFAs function as “building blocks” for even longer fatty acids that have a direct positive effect for the production of healthy sperm. The invention hence provides a composition comprising VLCFAs for use in treatment of a male subject's ability for production of healthy sperm wherein an increased concentration of VLCFAs in specific tissue related to the testis and spermatozoa is obtained. Hence, the treatment may enhance the function and/or viability of the sperm, or to increase the amount of mature sperm cells. Similar misunderstandings, as represented by the publications by Gorusupudi et al. (related to age macular degeneration) and Giannaccare et al. (related to dry eyes disease), appear to be present in studies performed to study the effect of omega-3 supplements on testicular function and male fertility. According to a review by Esmaieili et al. (Esmaeili, V., Shahverdi, A. H., Moghadasian, M. H. and Alizadeh, A. R. (2015) Dietary fatty acids affect semen quality: a review. Andrology 3: 450-461), inadequate DHA concentration is the main cause of low-quality spermatozoa (page 453, first column). In contrast to other PUFA-rich tissues, such as the brain and retina, the testis is continuously drained of PUFAs (such as DHA), as the spermatozoa are transported to the epididymis. However, three published studies with DHA supplements appear to have given conflicting results:

### i) Two Studies Utilising Fish Oil Derived High DHA Concentrates Reported Positive Results Parameters of Male Fertility

Martinez-Soto J C, Domingo J C, Cordobilla B, et al. (Dietary supplementation with docosahexaenoic acid (DHA) improves seminal antioxidant status and decreases sperm DNA fragmentation. Syst Biol Reprod Med. 2016;62(6): 387-395. doi:10.1080/19396368.2016.1246623) utilised a fish oil derived 76% DHA concentrate. The authors found that dietary supplementation with this DHA product induced an increase of omega-3 fatty acids and DHA concentration in seminal plasma, associated with the increase in total antioxidant capacity and a lower sperm DNA. After 10 weeks of supplementation, the percentage of spermatozoa with DNA damage was reduced from 22.0% to 9.3%. In contrast, placebo supplementation with sunflower oil did not induce any change in seminal parameters. Marinez et al. appears to have utilised a DHA concentrate similar in fatty acid composition to the commercially available DHA concentrate in study number 15 from the publication of Giannaccare et al., where chemical analyses in the applicant's laboratory proved the presence of small amounts of VLCPUFAs, please see discussion above related to the analysis of the Giannaccare et al. meta-study on dry eyes disease.

Gonzalez-Ravina C, Aguirre-Lipperheide M, Pinto F, et al. (Effect of dietary supplementation with a highly pure and concentrated docosahexaenoic acid (DHA) supplement on human sperm function. Reprod Biol. 2018; 18 (3): 282-288. doi:10.1016/j.repbio.2018.06.002) similarly utilised a high DHA concentrate (NuaDHA, containing 85% DHA according to the manufacturer (https://nuabiological.com/nua-dha/nua-dha-composicion-e-ingredientes/), which from the disclosures of the present application indicates a presence of VLCPUFAs. The authors conclude that “their study support previous indications that highlights the importance of DHA supplementation as a means of improving the sperm quality in asthenozoospermic men” (abstract). An outcome of the study was a clear indication of DHA supplementation for patients with asthenozoospermia, suggesting that dietary DHA supplementation at 1 g/day would be particularly beneficial for this infertile population (discussion section, final paragraph).

### ii) Study Utilising DHA-Rich Algal Oil

A publication by Conquer et al (Conquer J A, Martin J B, Tummon I, Watson L, Tekpetey F. Effect of DHA supplementation on DHA status and sperm motility in asthenozoospermic males. Lipids. 2000; 35 (2):149-154. doi:10.1007/BF02664764) utilised a microalgal oil, containing 38.6% DHA.

The authors state that seminal plasma phospholipid DHA levels are lower in asthenozoospermic men than normozoospermic. Their study showed that their DHA supplementation increases levels of this fatty acid in seminal plasma to levels comparable with those reported previously in normozoospermic men. However, although DHA supplementation did modify levels of this fatty acid in serum and seminal plasma, supplementation had no effect on DHA levels in the spermatozoa, and the DHA supplementation had no effect on sperm motility in the asthenozoospermic men. According to the authors, the absence of effect on DHA levels in the spermatozoa is more likely to be related to an inability of the sperm to take up preformed DHA. In this respect, the authors refer to one study in normozoospermic humans which “suggests that DHA levels rise with supplementation by fish oil (a source of EPA+DHA)”.

None of the three publications referred to above mentions VLCPUFAs. However, based on what is disclosed in the present application, the inventors realise that in addition to DHA, the supplementation of VLCn3s is vital in order to obtain healthy spermatozoa. The two first studies, with positive results as to improving sperm quality, utilised DHA concentrates from fish oils, which also would have contained small amounts of VLCn3s. The third study, which did not document any affect om sperm quality, utilised an algal oil, which is not known to contain VLCn3s with structures useful as “building blocks” as disclosed in the present application. The inventors of the present invention have realised that, even though the fatty acid DHA may have a role for sperm quality, there is also a need for VLCFAs, and that such VLCFAs can be provided by compositions according to the present invention. As shown by the examples of the present invention, it has very surprisingly been found that compositions of VLCFAs, which have been added to the feed, can be absorbed and transported to testis tissue (Example 1A). Thus, supplemented VLCFAs that are beneficial for the male fertility can reach testis, and there perform their functions. This means that supplementation with compositions of VLCFAs according to the present invention can be utilised in treatment of male fertility, such as reduced function and/or viability of the sperm, or a reduced amount of mature sperm cells, such as of an individual who has developed a reduced ability for in vivo synthesis of VLCFAs.

### Cognitive Health

In a yet another embodiment, the invention provides a composition for use for treatment of a subject's disease related to the brain and nervous tissues. Such as wherein the subject has deficiencies in one or more endogenous elongase systems that are important for a healthy brain and nervous tissues, by introducing to the subject a lipid composition comprising VLCFAs. This may have a direct positive health effect or the VLCFAs function as “building blocks” for even longer fatty acids that have a direct positive effect for the healthy brain or nervous tissues. The invention hence provides a composition comprising VLCFAs for use in treatment of a disease related to the brain and nervous tissue wherein an increased concentration of VLCFAs in the specific tissue is obtained.

Low consumption of the omega-3 fatty acids EPA and DHA has been linked to delayed brain development, and, in later life, increased risk for reduced cognitive performance, including increased risk for Alzheimers disease (AD). However, published studies in this field appear to show conflicting results. It appears to be well established that fish consumption is beneficial for healthy cognitive performance. Albanese et al. (Dietary fish and meat intake and dementia in Latin America, China, and India: a 10/66 Dementia Research Group population-based study, Am J Clin Nutr 2009; 90:392-400), in a study based on 14960 residents aged 65 years in China, India, Cuba, the Dominican Republic, Venezuela, Mexico and Peru, and by performing meta-analysis combining data from all the countries, found a significant association between lower prevalence of dementia and higher dietary fish intake.

Freund-Levi et al. (ω-3 Fatty Acid Treatment in 174 Patients With Mild to Moderate Alzheimer Disease: OmegAD Study, A Randomized Double-blind Trial, Arch Neurol. 2006; 63:1402-1408), found that administration of omega-3 fatty acids gave positive results in the sub-group of patients with very mild AD. Combined with data from epidemiologic studies, which suggest that the risk for development of AD is reduced by fish consumption, Freund-Levi et al. concluded that their study supported the idea that omega-3 fatty acids have a role in primary prevention of AD, but not in treatment of manifest disease. Freund-Levi et al. utilised supplementation with 2.8-fold more DHA than EPA and randomised the patients to either receive four 1 g capsules daily, each containing 430 mg DHA and 150 mg EPA (EPAX1050TG of the applicant), or corn oil as an isocaloric placebo. EPAX1050TG is a concentrate of DHA obtained from fish oils. As discussed below, this concentrate also contains some amounts of VLCFAs.

Kongai et al. (Effects of krill oil containing n-3 polyunsaturated fatty acids in phospholipid form on human brain function: a randomized controlled trial in healthy elderly volunteers,

Clinical Interventions in Aging 2013:8 1247-1257) performed a study where males, aged 61-72 years, received 12 weeks of treatment with: medium-chain triglycerides as placebo; krill oil, which is rich in n-3 PUFAs incorporated in phosphatidylcholine; or sardine oil, which is abundant in n-3 PUFAs incorporated in triglycerides. Changes in oxyhemoglobin (oxy-Hb) concentrations in the cerebral cortex during memory and calculation tasks were measured, and the authors found that the during the working memory task, changes in oxy-Hb concentrations in the krill oil and sardine oil groups were significantly greater than those in the placebo group at week 12. Krill oil gave the best results, motivating the authors to conclude: “This study provides evidence that n-3 PUFAs activate cognitive function in the elderly. This is especially the case with krill oil, in which the majority of n-3 PUFAs are incorporated into phosphatidylcholine, causing it to be more effective than sardine oil, in which n-3 PUFAs are present as triglycerides”.

The participants in the study by Kongai et al. received 2 grams (8×0.25 g capsules) of the respective oils per day, representing 193 mg EPA and 92 mg DHA pr. day for the krill oil (i.e.

96.5 mg EPA and 46 mg DHA per gram krill oil), and 491 mg EPA and 251 mg DHA for the “sardine oil” (i.e. 245.5 mg EPA and 125.5 mg DHA per gram “sardine oil”). The skilled person realises that 245.5 mg/g EPA and 125.5 mg/g DHA (sum 371 mg/g (EPA+DHA), and a total of 460 mg/g omega-3 acids, are values significantly higher than what can be found in natural fish oils, and that the so-called sardine “SO” oil therefore represents a product containing moderately up-concentrated C20-C22 omega-3 fatty acids derived from fish oil. As shown in the discussion below, overlooked small amounts of VLCFAs, which are components in krill oil, and in the moderately concentrated omega-3 fatty acids that were utilised by Kongai et al., represent fatty acids that can be surprisingly important factors for maintaining a healthy cognitive performance. As mentioned above, Kongai et al. utilised increased oxy-Hb concentrations in the cerebral cortex, during memory and calculation tasks, as a measure of increased cerebral blood flow. Such a procedure had earlier been utilised by Jackson et al. (DHA-rich oil modulates the cerebral haemodynamic response to cognitive tasks in healthy young adults: a near IR spectroscopy pilot study, British Journal of Nutrition (2012), 107, 1093-1098), who found that supplementation with “DHA-rich FO”, in comparison with placebo, resulted in a significant increase in the concentrations of oxy-Hb and total levels of haemoglobin (Hb), indicative of increased cerebral blood flow (CBF), during the cognitive tasks. In comparison, no effect on CBF was observed following supplementation with “EPA-rich FO”. As also the “EPA-rich FO” contained appreciable amounts of DHA (please see details below), the authors concluded that the CBF response “is only modulated following supplementation with DHA at a dose higher than 200 mg/d”. The acronym “FO” is used as an abbreviation for “fish oil”, which from the context means fish oil derived EPA and DHA. The treatment oils (see page 1094) were purchased from EPAX AS (Aalesund, Norway, i.e. the applicant of the current application), and encapsulated into 500 mg capsules. Based on the information given by the authors, the 2 daily 500 mg capsules of DHA and EPA rich oils had the following contents of EPA and DHA (contents which are enriched compared to natural fish oils):

“DHA-rich FO”: 450 mg DHA and 90 mg EPA (i.e. fairly similar to the 430 mg DHA and 150 mg EPA “EPAX1050TG” as utilised by Freund-Levi et al., in their article that is discussed above).

“EPA-rich FO”: 300 mg EPA and 200 mg DHA.

The above cited scientific publications give important information:
Published studies focus on the effect of the marine omega-3 fatty acids EPA and DHA. However, the studies appear to show conflicting results.
Fish consumption appears to be beneficial for healthy cognitive performance. Krill oil appears to be beneficial for healthy cognitive performance.
Fish oil omega-3 concentrates with a high content of DHA compared to EPA appear to give positive results.
Fish oil omega-3 concentrates with a high content of EPA compared to DHA appear to give results that are less positive than fish oil omega-3 concentrates with a high content of DHA compared to EPA.

The latter statement appears to be somewhat contrary to the fact that most natural fish oils, as well as krill oil, contain more EPA than DHA. Further, the amounts of DHA utilised to obtain positive results vary very much: Freund-Levi et al. utilised a daily dose of 1.7 g DHA. Jackson et al. concluded that increased cerebral blood flow during the cognitive tasks is only modulated following supplementation with DHA at a dose higher than 200 mg/day. Kongai et al., utilising very similar assessment procedures as Jackson et al., obtained significant positive results with DHA supplementation of only 92 mg/day.

However, in addition to DHA, and the possible preferential role of DHA in phosphatidylcholine, the inventors of the present invention have realised that VLCFAs, a completely overlooked group of fatty acids in these studies, have surprisingly important roles, explaining conflicting results in the scientific literature:

Natural fish oils and krill oil contain small valuable amounts of VLCFAs. Concentrates of marine omega-3 fatty acids, as illustrated by the above cited scientific publications, focus on concentrating the fatty acids EPA (C20:5n3) and DHA (C22:6n3). In order to obtain such concentrates, for examples by molecular/short path distillation or extraction procedures, components with molecular weight less than that of EPA, and above that of DHA, have typically been removed. Especially, it has been desired to remove components with molecular weight above that of DHA in order to get rid of high molecular weight impurities, like oligomers formed by oxidation/decompositions of the easily oxidised and heat sensitive marine LCPUFAs. As unsaturated fatty acids are very liable to oxidation, and in order to comply with pharmacopoeia and voluntary standards imposing upper limits for oligomeric/polymeric oxidation products, components with chain lengths above that of DHA have commonly been removed, for example by distillation, extraction and similar procedures. Further, such higher molecular weight components of marine oils are typically associated with undesirable unsaponifiable constituents of such oil including cholesterol and organic pollutants such as brominated diphenyl ethers. Unfortunately, the removal of unwanted heavy components has also meant that a large fraction of the valuable VLCFAs originating from the starting natural oils also have been removed.

The removal of VLCFAs has especially been the case during production of concentrates that are highly enriched in EPA, and where, for this reason, also a part of the C22 fraction, which includes DHA, is removed. On the other hand, when manufacturing concentrates of DHA, the inventors of the present invention have found that appreciable amounts of VLCFAs can remain in the product. For example, when analysing an existing concentrate containing 50% DHA, 6% DPA and only 8.5% EPA, the applicant found that this product contained 1.4% C24-C30 VLCn3s. [Giannaccare et al., study No. 15]. When analysing a sample from a batch of DHA-rich EPAX1050 TG, the applicant found a content of 0.2% VLCPUFA and 0.6% VLCMUFA. The authors of different publications of studies using concentrates from natural oils have been silent as to the presence or effect of VLCPUFAs/VLCn3s from the natural oil, and it is clear that the positive effects of VLCPUFAs from natural oil have not been apparent for the scientific community.

Moderately up-concentrated products of EPA plus DHA from natural oils may also contain small amounts of VLCFAs, as these concentrates normally have been manufactured by the removal of a limited fraction of the fatty acids above that of DHA.

When analysing a commercially encapsulated krill oil, the applicant found a content of 0.2% C24-C30 VLCPUFAs and 0.2% VLCMUFAs. However, as commercial krill oil production methods appear to be based on several quite different production methods, the exact concentration of VLCFAs in krill oils in the market may vary somewhat compared to these values.

Thus, in the article of Jackson et al., the “DHA-rich FO” will have contained significantly higher relative concentrations of VLCFAs than the “EPA-rich FO”, positively influencing the cerebral blood flow during cognitive tasks. Similarly, in the article of Kongai et al., it is likely that the SO omega-3 fish oil concentrate contained less VLCFAs than the krill oil, contributing to the krill oil positive results, even though the krill oil contained far less EPA and DHA than the SO oil.

The brains of higher animals, and particularly myelin, contain VLCFAs. The concentration of VLCFA in the brain increases with development. The brain and myelin contain saturated and monounsaturated, as well as polyunsaturated VLCFAs. The normal young human brain contains polyunsaturated VLCFAs with at least up to 38 carbon atoms. α-Hydroxy VLCFAs also occur in the brain. (A Poulos (1995) Very long chain fatty acids in higher animals—a review, Lipids, 30: 1-14.)

According to Steinberg et al., in humans, one specific very long-chain acyl-CoA synthetase (VLCS) is expressed preliminary in the brain (Steinberg S J, PA (2000) Very Long-chain Acyl-CoA Synthetases. Human “Bubblegum” represents a new family of proteins capable of activating very long chain fatty acids, Journal of Biological Chemistry, 275, No. 45, pp. 35162-35169). The concentration of VLCFAs increases during development, and these VLCFAs are components of complex lipids such as gangliosides, cerebrosides, sulfatides, sphingomyelin, and other phospholipids. Activation by VLCSs is required for incorporation of VLCFAs into these complex lipids. Many of these VLCFA-containing lipids are components of myelin membranes in the brain.

The inventors of the present invention realised that if, for example from reasons related to ageing, an individual's ability for in vivo synthesis of valuable brain VLCFAs, or for incorporation of VLCFAs into complex lipids, is reduced, supplementation of compositions according to the present invention could ameliorate the subsequent negative effects on the individual's cognitive health.

This surprising disclosure is in strong contrast to the state of the art. As an example, in a very recent review article on algae for production of omega-3 acids, the author is completely silent as to VLCPUFAs as defined by the present invention. (Harwood J L, Review: Algae: Critical Sources of Very Long-Chain Polyunsaturated Fatty Acids, Biomolecules 2019, 9, 708; doi:10.3390/biom9110708). According to Harwood, “there is a lot of evidence that dietary EPA and DHA have beneficial effects for good health”, and these benefits include improved brain function (Introduction, last paragraph). As shown by the text and tables, there is no mention of production or use of fatty acids with chain length above C22.

In contrast to this view, the inventors of the present invention have realised that, even though the fatty acids DHA and EPA are very important for brain functions, there is also a need for VLCFAs, and that such VLCFAs can be provided by compositions according to the present invention. As shown by the examples of the present invention, it has very surprisingly been found that compositions of VLCFAs, which have been added to the feed, can be absorbed and transported to the brain (Example 1A, 2B, 3). Thus, supplemented VLCFAs that are beneficial for the cognitive health can reach the brain in order to be incorporated into inter alia myelin, and there perform their functions. This means that supplementation with compositions of VLCFAs according to the present invention can be utilised as treatment to ameliorate the negative effects on the cognitive health, such as of an individual who has developed a reduced ability for in vivo synthesis of VLCFAs.

In a further embodiment, the invention discloses a composition for use for treatment of a subject's disease related to the skin and/or endothelial and mucosal tissues/mucous membrane, wherein the subject has deficiencies in one or more endogenous elongase systems that are important for healthy skin and/or endothelial and mucosal tissues, by introducing to the subject a lipid composition comprising VLCFAs. This may have a direct positive health effect or the VLCFAs function as “building blocks” for fatty acids that have a direct positive health effect for healthy skin and/or endothelial and mucosal tissues/mucous membrane. The invention hence provides a composition comprising VLCFAs for use in treatment of a disease of the skin and/or endothelial and mucosal tissues/mucous membrane, wherein an increased concentration of VLCFAs in such specific tissue is obtained. In one embodiment, the composition for use includes treatment of one or more of diseases of the skin and/or endothelial and mucosal tissues/mucous membrane, for example dry skin, eczema and allergy. Reference is made to the Examples. Examples 1A and 2B show uptake of VLCPUFAs in skin tissue. Examples 5 and 6 show positive effects of VLCFAs on wound healing, and in promoting thicker epidermis and improved scale development. In another embodiment, the composition for use encompass treatment of the lungs and respiratory tract such as asthma.

To summarize, the composition for use may be used in treatment of one or more of the following diseases:
i) eye diseases, such as macular degeneration (AMD), diseases caused by diabetic inflammation of the eye, and dominant Stargardt macular dystrophy (STGD3);
ii) male fertility, such as reduced function and/or viability of the sperm, or a reduced amount of mature sperm cells;
iii) skin and endothelial diseases, including any of dry and wrinkled skin, irritated, sour or sensitive skin, ability for wound healing, protecting against negative effects on the skin from the sun's UV radiation, negative effects on hair follicles, reduced hair health including risk of hair loss, including eczema, psoriasis, acne and rosacea;
iv) diseases of mucosal tissue/mucous membranes, such as lung diseases, diseases of the respiratory tract including asthma, liver diseases, and allergy, diseases of the urine system and digestive system;
v) diseases of the brain and nervous tissue, including the central nervous system, such as reduced mental health, demyelinating diseases such as multiple sclerosis, Parkinson's, Schizophrenia, Dementia, Alzheimer's, impaired cognitive function, migraine, seizures and epilepsy;
vi) inflammatory related diseases as cardiovascular diseases such as e.g. atherosclerosis and rheumatoid arthritis.

The invention further provides a method to increase the blood levels of VLCFAs in subjects, particularly in subjects having a reduced ability for endogenic synthesis of VLCFAs, such as those having an inefficient elongase system. The increase or correction of VLCFAs achieved by use of the method or composition of the invention can be quantified as a VLCFA enrichment in blood, such as in red blood cells (erythrocytes) or in blood plasma. Further, the invention provides a method for increasing or normalizing the level of VLCFAs in the specific tissue involving the disease to be treated. Particularly, as shown in the Examples, the applicant has studied uptake of VLCFAs in specific tissues of animals (mice, salmon, rats) which have been fed with diets comprising VLCFAs, and has found that the VLCFAs can be quantified as VLCFA enrichments in specific tissues. In one group of studies, salmon and rats have been fed with marine oils, and the applicant has analysed tissue of the eyes, brain, testis, liver, heart and skin to identify that VLCFAs are taken up by these tissues. Analysis and quantification of the fatty acids present in the tissue can be done according to the art, e.g. in vitro by chromatography, often coupled with mass spectrometry, after having extracted the relevant tissue with an appropriate solvent. The included Examples provide data that show that the content of VLCFAs in several tissue types and types of animals/fish can be directly influenced by supplementation of VLCFAs. There is a direct uptake of the VLCFAs from the administered VLCFA-comprising compositions, such as from the diet. Previous studies have shown that there are elongases and desaturases which are responsible for the formation of VLCPUFAs. The applicant has however now found an alternative way to obtain these “essential” fatty acids into different tissues. The examples of the present application clearly demonstrate that VLCFAs are taken up from the digestive tract, and transported to various tissues, like the liver, skin, brain, retina, eyeball, and also in blood plasma. When comparing to control diets, with similar fatty acid compositions, except for the VLCFAs, it is clearly demonstrated that the observed increase of VLCFAs in the tissues is not just a result of in vivo synthesis from fatty acids with shorter fatty, e.g. like in vivo synthesis from LCPUFAs. In the studies of the Examples, the VLCFAs have been administered orally, by including them in feed. Alternative administration routes are provided below.

In one embodiment, the invention provides a method to increase the level of VLCFAs or to correct a deficiency of VLCFAs in subjects' blood, particularly in subjects having a reduced ability for endogenic synthesis of VLCFAs. By the composition for use, a substantial increase in the amount of VLCFAs in the blood plasma is achieved. Further, the invention provides a method as disclosed to correct an imbalance in the ratio of LCPUFAs to VLCPUFAs in the blood. In one embodiment, the change obtained e.g. in erythrocyte VLCFA, as a percentage of total fatty acids, by using the method of the invention is at least 10 percent, such as at least 20 percent, such as e.g., a 30-60 percent increase. Alternatively, quantitative measurements can be made of the actual erythrocyte VLCFAs. By the composition for use, a substantial increase in the amount of VLCFAs in the blood is achieved. In one embodiment, the invention provides a method to increase the level of VLCFAs or to correct a deficiency of VLCFAs in subjects' blood, particularly in subjects having a reduced ability for endogenic synthesis of VLCFAs. Further, the invention provides a method as disclosed to correct an imbalance in the ratio of LCPUFAs to VLCPUFAs in the blood. By the composition for use, a substantial increase in the amount of erythrocyte VLCFAs is achieved. In one embodiment, the invention provides a method to increase the level of VLCFAs or to correct a deficiency of VLCFAs in subjects' tissues, particularly in subjects having a reduced ability for endogenic synthesis of VLCFAs. Further, the invention provides a method as disclosed to correct an imbalance in the ratio of LCFAs to VLCFAs in the tissue. The tissue is e.g. selected from the group of the eyeball, retinas or meibum, sperm and testes, brain and nervous systems, epidermal and mucosal membranes/tissues, including tissues of the lung and respiratory tract, tissue of the cardiovascular system, and of the urine bladder, urinary system, digestive system.

### Composition

The VLCFAs of the lipid composition belong to one or more of the fatty acid groups

VLCPUFAs, i.e. either including, but not limited to VLCn3 and VLCn6, or, VLCMUFAs, including VLCMUFAn7, VLCMUFAn9, VLCMUFAn11, VLCMUFAn13, and VLCSAs. In one embodiment, the lipid composition for use in the treatment of the invention comprises at least 5% by weight of VLCFAs. In some (preferred) embodiments the main components of the VLCFAs are omega-3 acids and/or monounsaturated fatty acids. The fatty acids are obtained from, i.e. are isolated from, a natural source, such as from a marine oil as detailed below.

Hence, the invention provides a composition comprising at least 5% by weight of VLCFAs for use in treatment of a disease of a subject, particularly wherein the disease is associated with a deficiency in one or more endogenous elongase systems and/or with a reduced ability for endogenic synthesis of VLCFAs.

In one embodiment, the lipid composition comprises at least 4.0% by weight of very long chain monounsaturated fatty acids and at least 1.0% by weight of very long chain polyunsaturated fatty acids. In another embodiment, the lipid composition comprises at least 1.0% by weight of very long chain monounsaturated fatty acids and at least 4.0% by weight of very long chain polyunsaturated fatty acids.

Further, in one embodiment the lipid composition comprises at least 8% by weight of VLCMUFAs, such as at least 15% by weight of VLCMUFAs.

In one embodiment, the lipid composition comprises at least 2% by weight of VLCPUFAs, such as at least 5% VLCPUFAs. The VLCPUFAs are preferably omega-3 or omega-6 fatty acids. For some specific uses, such as therapy of male fertility, the composition comprises omega-6 VLCPUFAs.

In one embodiment, the lipid composition comprises at least 8%, 10%, 12%, 15%, such as at least 20%, at least 25%, and more preferably at least 30% by weight of very long chain fatty acids in total.

In one embodiment, the composition comprises a mixture of several different fatty acids, of various lengths and degree of unsaturation. Such composition may comprise at least two different VLCFAs, such as at least three different VLCFAs. In one embodiment, the composition comprises LCPUFAs in addition to VLCFAs, as further disclosed below. E.g. the composition comprises at least two LCPUFAs and at least two VLCFAs. Further, the composition may comprise both omega-3 and/or omega-6 VLCPUFAs and also VLCMUFAs. In one embodiment, the composition comprises either of omega-3 and omega-6 VLCPUFAs with more than 6 double bonds.

VLCFAs that may be present in the compositions are selected from any one of, including but not limited to, the following group of fatty acids:
C24:1n9 (nervonic acid) and other isomers of tetracosenoic acid);
C26:1n9 and other isomers of hexacosenoic acid;
C28:1n9 and isomers of octacosenoic acid)
C30:1, C32:1, C32:1 and even longer monounsaturated fatty acids
C24:4n3, C24:5n3, C24:6n3, particularly C24:5n3;
C26:3n3, C26:4n3, C26:5n3, C26:6n3, C26:7n3, particularly C26:6n3;
C28:3n3, C28:4n3, C28:5n3, C28:6n3, C28:7n3, C28:8n3, particularly C28:7n3, C28:8n3;
C30:3n3, C30:4n3, C30:5n3, C30:6n3; C30:7n3, C30:8n3;
C32:3n3, C32:4n3, C32:5n3, C32:6n3, C32:7n3, C32:8n3, C32:9n3, particularly C32:7n3, C32:8n3;
C34:4n3, C34:5n3, C34:6n3, C34:7n3, C34:8n3, C34: 9n3, particularly C34:7n3, C34:8n3;
C36:4n3, C36:5n3, C36:6n3, C36:7n3, C36:8n3, C36: 9n3, particularly C36:7n3, C36:8n3, or even longer omega-3 fatty acids;
C24:2n6, C24:4n6, C24:5n6,
C26:4n6, C26:5n6, C26:6n6;
C28:4n6, C28:5n6, C28:6n6, C28:7n6;
C30:4n6, C30:5n6, C30:6n6; C30:7n6
C32:4n6, C32:5n6, C32:6n6, C32:7n6, C32:8n6,
C34:4n6, C34:5n6, C34:6n6, C34:8n6
C36:4n6, C36:5n6, C36:6n6, C34:8n6 or even longer omega-6 fatty acids;
and may also contain the VLCSAs C24:0, C26:0, C28:0, C30:0 or C32:0, or even longer saturated fatty acids.

In certain embodiments the compositions for use according to the invention may contain some amount of fatty acids with even longer chain length than C32, i.e. including, but not limited to, fatty acids with chain length C34, C36, C38 and C40. Further, other positional isomers of the fatty acids listed above, and fatty acids with a different number of fatty acids, and/or a different number of double bonds than listed above, may be present in the compositions.

The Examples show that VLCFAs from administered compositions are taken up in different tissues and in blood plasma. In one embodiment, the composition for use comprises any of the fatty acids shown in the Examples to be taken up. The dominating fatty acids present in the feed, are particularly those with greatest increase in the tissues. Particularly, in one embodiment the composition for use comprises at least one of the fatty acids selected from the group of C24:5n3, C26:6n3 and C28:8n3.

In diseases wherein certain VLCFAs are known to build up, such fatty acids should not be included in composition for use in the treatment.

In one embodiment, the composition comprises at least 4% by weight of a VLCMUFA with the chain length of C24-C32, and in one embodiment the composition comprises the VLCMUFA C24:1. Particularly, for treatment of some diseases related to brain and nervous tissues, it may be beneficial to include a high concentration of this fatty acid. However, in diseases wherein VLCMUFAs are known to build up, such fatty acids should not be included in the treatment. In one embodiment, the method comprises the step of administering a lipid composition comprising the C24:1 fatty acid in an amount of 4.0-50.0%, such as 7.0-40.0%, 8.0-20.0%, such 13.0-20.0%, such as about 40%. Even further, for the treatment of diseases of the brain and nervous tissues, and also for eye health and pre- and postnatal health, the composition preferably comprises a high concentration of DHA. As shown in Example 2B, related to uptake in brain tissue, the fatty acid C28:8 is absorbed more than others in the brain tissue, supporting that this fatty acid may be included in compositions for brain health.

The fatty acids of the administered lipid composition, and according to the above embodiments, may be present in the form of free fatty acids, free fatty acid salts, mono-, di-, triglycerides, ethyl esters, wax esters, (O)-Acetylated ω-hydroxy fatty acids (OAHFAs), cholesteryl esters, ceramides, phospholipids or sphingomyelins, alone or in combination. Or, the fatty acids may be in any form that can be absorbed in the digestive tract, or that can be absorbed by specific tissue by local application. Preferably, the fatty acids are in the form of free fatty acids, fatty acid salts, ethyl esters, glycerides or wax esters. For local applications delivering preparations comprising the VLFAs compositions, the fatty acids are preferably in the form of free fatty acids, fatty acid salts, as glycerides (mono- di- or triglycerides alone or in combinations), OAHFAs, cholesteryl esters, ceramides, phospholipids, sphingomyelins or wax esters, and in an even more preferred embodiment the VLCFAs are in the form of wax esters. In one embodiment, for the local application of the composition, this comprises salts, and accordingly at least some of the fatty acids of the composition, such as at least some of the VLCPUFAs, may be in the form of fatty acid salts.

In addition to the VLCFAs, the lipid composition for use may further comprise other fatty acids, such as further long chain polyunsaturated fatty acids. In one embodiment, the composition for use comprises at least 5% by weight of one or more LCPUFA, such as one or more C20-C22 PUFAs. In certain embodiments, such compositions of this invention comprise at least 10 percent, at least 25 percent, at least 30 percent, at least 40 percent, at least 50 percent, at least 60 percent, or at least 70 percent by weight of at least one LCPUFA, such as one or more C20-C22 long chain PUFAs. In one embodiment, the LCPUFAs comprise at least one of EPA, DHA and omega-3 DPA (n3DPA, all-cis-7,10, 13, 16,19-docosapentaenoic acid). In a further embodiment, the compositions of this invention comprise at least 5 percent, at least 10 percent, or at least 20 percent, at least 30 percent, at least 40 percent by weight of DHA. Further, in other embodiments, the compositions of this invention comprise at least 5 percent, at least 8 percent, or at least 10 percent by weight of DPA (22:5n3). In some embodiments of the present invention, the weight ratio of EPA:DHA of the composition ranges from about 1:15 to about 10:1, from about 1:10 to about 8:1, from about 1:8 to about 6:1, from about 1:5 to about 5:1, from about 1:4 to about 4:1, from about 1:3 to about 3:1, or from about 1:2 to about 2:1. In one embodiment, the composition for use comprises 5-30% VLCFAs and 50-90% LCPUFAs, by weight of the composition. In one embodiment, the lipid composition for use comprises mainly fatty acids and/or fatty acid derivatives, and preferably at least 90.0%, such as at least 95.0% by weight of the lipid composition is fatty acids.

Further, in some embodiments the lipid composition enriched with VLCFAs comprises a further amount of monounsaturated fatty acids. In one embodiment, the composition for use comprises at least 5% by weight of one or more LCMUFA, such as one or more C20-C22 MUFAs. In certain embodiments, such compositions of this invention comprise at least 10 percent, at least 25 percent, at least 30 percent, at least 40 percent, at least 50 percent, at least 60 percent, or at least 70 percent by weight of at least one LCMUFA, such as one or more C20-C22 long chain MUFAs. In some embodiments the composition enriched with VLCFA also comprises an amount of C18 MUFA, such as C18:1n9 and/or C18:1n7.

Further, in some embodiments the lipid composition enriched with VLCFAs comprises a low amount of saturated fatty acids, of all lengths. In total, the composition comprises less than 1.0% saturated fatty acids, more preferably less than 0.5% saturated fatty acids. Particularly, the amount of C16:0 (palmitic acid), C18:0 (stearic), and C20:0 (arachidic acid) is low, and preferably the content of these, in total, is less than 1.0%. Particularly, the amount of stearic acid is low, and is preferably below 1.0%, and more preferably below 0.5%. Further, the amount of very long chain saturated fatty acids (VLCSFA) is low, and the amount of the fatty acids C24:0, C26:0, C28:0 and C30:0 is preferably in total below 2.0%, more preferably below 1.0% and most preferably below 0.5% by weight of the fatty acid mixture. However, in other embodiments, e.g. wherein the composition is for use in therapy of the skin or mucosa, the composition may comprise very long chain saturated fatty acids (VLCSAs). E.g. the composition comprises more than 1.0%, such as more than 2.0% of VLCSAs, and relevant VLCSAs to include in the composition for use are e.g. lignoceric acid (C24:0) and cerotic acid (C26:0). In one example, the composition comprises C24:0 and is for treatment of skin diseases and particularly papulopustular rosacea.

Bennett and Anderson (2016) (Current Progress in Deciphering Importance of VLC-PUFA in the retina. In: C. Bowes Rickman et al. (eds.) Retinal Degenerative Diseases, Advances in Experimental Medicine and Biology 854, Springer, Switzerland), in a book chapter on current progress in deciphering importance of VLCPUFA in the retina, state the importance of these fatty acids would be solidified if VLCPUFAs could be reconstituted in the deficient retinas.

“However, VLCPUFAs cannot be chemically synthesised in large enough quantities to allow feeding studies in mice”. This belief is stated despite much work has focused upon synthetic production of VLCPUFAs using recombinant techniques. For example, Anderson et al (US 2009/0203787A1, US 2012/0071558A1 and US 2014/0100280A1) disclose a recombinant process for producing C28-C38 VLCPUFAs using the ELOVL4 gene, and Anderson et al. indicate (in paragraph 13 of US 2009/0203787A1) that such recombinant processes are necessary as VLCPUFAs are only naturally found in extremely small quantities in a few organs or certain animal species, stating that “In order to obtain even minute μg quantities of these VLC-PUFAs, they must be extracted from natural sources such as bovine retinas. As a result, research into C28-C38 VLCPUFAs has been limited, and means for commercial production thereof have been non-existent.” Further, Raman et al. (US2013/0190399) discloses chemical synthesis of VLCPUFAs. According to Raman, [0009] “due to the limited enzymatic production rate and the limited amount of VLC-PUFAs found in the few known biological sources, study of the compounds and their therapeutic usefulness has been very limited. Therefore, there is a need for reliable and efficient chemical methods for producing VLCPUFAs . . . ”. In [0010]: Raman states: «Conventional sources of VLCPUFAs, such as retina, brain and sperm, have only extremely small amounts of these long chain fatty acids. Raman et al. start their synthesis from C20-C22 LCPUFAs such as DHA or DPA. By chemical synthesis using a “saturated zinc extender reagent” or an aldehyde the selected LCPUFAs are chemically attached to a separate chain of carbon atoms, not present in the oil, to provide synthetic VLC-PUFAs. Unfortunately, the disclosed chemical reaction between LCPUFAs such as EPA, DHA and DPA with a separate, non-natural, chain of carbon atoms via the synthetic “extender reagents” will lead to synthetic VLCPUFAs with the same number of double bonds as in the original PUFAs, i.e. 5 double bonds if starting with EPA and DPA, and 6 double bonds if starting with DHA. Raman discloses the synthesis of VLCPUFAs with 4,5 and 6 double bonds, and thus does not teach how to synthesise all the biologically important VLCPUFAs with varying number of double bonds.

In nature the double bonds of fatty acids are all in the cis-form. In polyunsaturated omega-3 and omega-6 fatty acids each double bond is separated from the next by one methylene (—CH2—) group. The all cis-form as well as the exact position of the double bonds in the fatty acid molecule are vital for the biological transformations and actions of the fatty acids. The polyunsaturated fatty acids of the composition for use are substantially all in the cis-form. The actions of the natural fatty acids in the body may set them apart from chemically synthesized fatty acids, which invariably contain some amounts of trans-isomers, as well as fatty acids where the position(s) of double bond(s) deviate from that of the beneficial natural fatty acids, including fatty acid isomers with conjugated double bonds. In the complicated biological reactions involving VLCPUFAs, including VLCn3s and VLCn6s, the trans and conjugated isomers would be transformed alongside the natural all-cis isomers, and result in molecules that would compete with and modify the biological effects of the natural fatty acid isomers.

In some embodiments, the fatty acids of lipid composition originate from, i.e. are isolated from, a natural source, such as from an oil from an aquatic animal or plant, a natural non-aquatic plant oil or a combination of such oils. Preferably, the fatty acids originate from an oil, or a combination of oils, from an aquatic animal or plant, such as from a marine or fresh water organism. More preferably, the fatty acids originate from a marine oil, i.e. an oil originating from a marine animal or plant. The marine oils may be selected from the list including, but not limited to, fish oil, mollusc oil, crustacean oil, sea mammal oil, plankton oil, algal oil and microalgal oil. The fatty acids of the lipid composition can also originate from a combination of two or more natural sources as described above. The term “fish oil” encompass all lipid fractions that are present in any fish species. “Fish” is a term that includes the bony fishes as well as the Chondrichthyes (cartilaginous fishes like sharks, rays, and ratfish), the Cyclostomata and the Agnatha. Without limiting the choice of raw materials, among the bony fishes preferred species can be found among fish of families such as Engraulidae, Carangidae, Clupeidae, Osmeridae, Salmonidae and Scombridae. Specific fish species from which such oil may be derived include herring, capelin, anchovy, mackerel, blue whiting, sand eel, cod and pollock. The oil can be derived from the whole fish, or from parts of the fish, such as the liver or the parts remaining after removing the fish fillets. Among the cartilaginous fish species, like sharks, the oil may preferably be obtained from the livers. The term “mollusc oil” includes all lipid fractions that are present in any species from the phylum Mollusca, including any animal of the class Cephalopoda, such as squid and octopus. The term “plankton oil” as utilised here, means all lipid fractions that can be obtained from the diverse collection of organisms that live in large bodies of water and are unable to swim against a current, not including large organisms such as jellyfish. The term “natural plant oils” is meant to include oil from algae and microalgae, and also meant to include oil from single cell organisms. Thus, the natural plant oils may be selected from all oils derived from non-transgenic plants, vegetables, seeds, algae, microalgae and single cell organisms. As employed herein, the terms “natural oil” and “oils from a natural source” means any fatty acid containing lipids, including, but not limited to one or more of glycerides, phospholipids, diacyl glyceryl ethers, wax esters, sterols, sterol esters, ceramides or sphingomyelins obtained from natural organisms. The natural organisms have not been genetically modified (non-GMO).

The VLCPUFAs of the lipid composition of the invention are substantially on the all-cis-form. The VLCFA composition for use according to the invention is hence substantially free from trans-fatty acids. The amount of trans isomers is less than 2%, less than 1%, such as less than 0,9 weight%, preferably less than 0.5 weight% and more preferably less than 0.3 weight% of total fatty acids. In one embodiment, the amount of trans isomers is in the range of 0.1-0.3 weight% of the oil, in another embodiment the amount of VLCFA trans isomers is in the range of 0.2-0.5 weight% of the oil. Thus, for optimal compositions, VLCFAs enriched from natural oils are more preferable from a biological point of view. The amount of trans fatty acids in a composition may be measured by, inter alia, a GC-FID method, wherein the trans fatty acids will appear right before, or right behind the main peak, and wherein they are assumed to have the same response factor as the all-cis fatty acids.

The fatty acid compositions according to the present invention may typically be obtained and isolated by suitable procedures for transesterification or hydrolysis of the fatty acids from the natural oil and subsequent physico-chemical purification processes. Compositions according to the present invention can, inter alia, be manufactured based on natural oils and methods according to those that are disclosed in patent application WO2016/182452, but are not limited to the starting oils and methods that are disclosed in that application. The fatty acids of the compositions for use are not chemically synthesized. The fatty acids of the lipid composition have been isolated and concentrated from the natural source to obtain an enriched amount of fatty acids. In one embodiment, the VLCFAs of the composition are unmodified as compared to the oil isolated from the natural source. Hence, in one embodiment, the chain length of the VLCPUFAs are unmodified, and preferably, the natural VLCPUFAs are included in the compositions, without any steps for elongations having taken place, prior to administration. Further, the compositions do not comprise any lipid producing cells that secrete or produce the VLCFAs. Rather, the compositions comprise a certain amount of VLCFAs, wherein these are isolated and up-concentrated from a natural source, using a method suitable for up-scaling and production for commercial use. Fatty acids are generally instable, and the fatty acids for use are to be prepared by methods wherein mild conditions are used (e.g. low temperature and pressure) to avoid degradation and isomerisation, e.g. to avoid that the natural all-cis-fatty acids are amended to trans-fatty acids or conjugated fatty acids.

The compositions for use may be included in different kinds of products and should be formulated according to the use. The compositions may be administered by any administration route, including but not limited to, orally, intravenously, intramuscularly, sublingually, subcutaneously, intrathecally, buccally, rectally, vaginally, ocularly, nasally, by inhalation, transdermally, and cutaneously. For oral use, the compositions presently disclosed may be formulated in variable forms, such as in oral administration forms, e.g., tablets or soft or hard capsules, chewable capsules or beads, or alternatively as a fluid composition. By intake of concentrates of the VLCFA fraction of the natural oils, the subjects benefit from higher positive effects, as well as much lower volume of medicine/supplement than by consuming natural oils like fish oil, krill oil, algal oil or calanus oil. At the same time the subject will benefit from the absence of caloric intake and potential negative effects of fatty acids and lipid components that do not promote alleviation and/or healing as disclosed in the present application.

In one embodiment of the invention, the administration of the lipid composition takes place via the oral route. In another embodiment of the invention, the administration of the lipid composition takes place via parenteral applications.

In a preferred embodiment the lipid composition for parenteral application is administered together with a diluent suitable for parenteral use, said diluent could be a lipid composition utilised for use as parenteral nutrition, i.e. being incorporated into a commercial lipid emulsion formulation, such as an intravenous fat emulsion used as a source of calories and essential fatty acids, e.g. Intralipid.

In one embodiment the treatment of diseases related to lung tissues and the respiratory tract takes place via inhalation devices according to the art.

In one embodiment the treatment of diseases related to the skin and mucosa takes place via transdermal delivery, such as by direct application to the skin and mucosa, such as by lotion or cream, or by patches, suppositories (and similar devices) according to the art. In another more general embodiment patches can be utilised to introduce the lipid composition into the body, for transdermal delivery of the fatty acids through the skin and into the bloodstream. Cosmetic products comprising compositions for use according to the invention include lotion and creams, skin hydrating formulations, sun protective formulations, and these are typically applied directly to the skin. In one embodiment, the composition is to be applied locally in or around the eyes or the eye lids. For local application, such preparation may be in the form of, for example, eye drops, ointments, salves, lotions, gels, ocular mini tablets and the like.

In some embodiments of the present disclosure, the composition acts as an active pharmaceutical ingredient (API), and the composition is for use as a medicament. In some embodiments, the fatty acids of the composition is present in a pharmaceutically-acceptable amount. As used herein, the term “pharmaceutically-effective amount” means an amount sufficient to treat, e.g., reduce and/or alleviate the effects, symptoms, etc., of at least one health problem in a subject in need thereof. In at least some embodiments of the present invention, the composition does not comprise an additional active agent. In this embodiment, the composition may be used in a pharmaceutical treatment of subject, such as of subjects diagnosed with a reduced ability for endogenic synthesis of VLCFAs. Relevant diseases are also disclosed above. In another embodiment, the composition according to the invention is a food supplement, nutritional supplement or dietary supplement comprising VLCFAs. In a related embodiment, the invention provides a composition selected from the group of Enteral Formulas for Special Medical Use, Foods for Specified Health Uses, Food for Special Medical Purposes (FSMP), Food for Special Dietary Use (FSDU), Medical Nutrition, and a Medical Food. Such a composition is particularly suited for subjects having a deficiency of certain nutrients, such as VLCFAs. The composition is suited for a nutritional management of subjects having a distinctive nutritional requirement. Such a composition is typically administered to the subject under medical supervision. The composition comprises the relevant VLCFAs, to increase or correct the level of the VLCFAs in the blood or in specific tissue, such as of a subject diagnosed with a reduced ability for endogenic synthesis of VLCFAs. Accordingly, the VLCFA-composition is particularly for treatment of a subject group with a reduced ability for endogenic synthesis of VLCFAs. The composition and the method of the invention have the ability to correct a nutritional deficiency in such a target population.

Dietary supplements according to the invention may be delivered in any suitable format, including, but not limited to, oral delivery, dermal delivery or mucosal delivery, including as eye drops. The ingredients of the dietary supplement can include acceptable excipients and/or carriers for oral consumption, and in particular in the form of an oral delivery vehicle, such as capsules, preferably gelatine capsules, liquids, emulsions, tables or powders.

### Dosage

The total daily dosage will depend on several factors, including which disease the subject has, severity of the disease, the subject, the composition, the formulation, type of use, and mode of administration. In one embodiment, the lipid composition dose is in the range from about 0.600 g to about 6.0 g. For example, in some embodiments, the total dosage of the composition ranges from about 0.8 g to about 4.0 g, from about 1.0 g to about 4.0 g, such as about 3.0 g, or from about 1.0 g to about 2.0 g. In case of using a highly concentrated VLCFA composition, with a concentration considerably higher than 5%, the dose might be much lower, for example around 0.06-0.6 g. The composition may be administered in from 1 to 10 dosages, such as from 1 to 4 times a day, such as once, twice, three times, or four times per day, and further for example, once, twice or three times per day. In one embodiment, the dose is adjusted according to the level of VLCFAs measured for the subject. The composition is preferably administered over a long period, such as 12-52 weeks, e.g. 24-46 weeks. An adequate level of VLCFAs is expected to be reached after 12-16 weeks, but the subject should continue the treatment to maintain this level. In one embodiment, the subject should continue to take the composition for the rest of the life.

### EXAMPLES

### Example 1

### Supplementation with VLCPUFA in Mice—Effect on Fatty Acid Composition of Eye (Eye Apple) and Blood Plasma

### Lipid Compositions

Lipidmix 1 and 2 were prepared from a standard anchovy fish oil. The crude fish oil was purified and ethylated, the ethylated oil was fractionated and up-concentrated by distillation and urea precipitation, and for Lipidmix 1 Lithium-precipitation was performed, to obtain the desired composition. The fractions were finally re-esterified to triglycerides by an enzymatic reaction with glycerol.

The fatty acid composition of Lipidmix 1 and 2 were analysed on a Scion 436-GC with a split/splitless injector (splitless 1 min), using a Restek Rxi-5 ms capillary column (length 30 m, internal diameter 0.25 mm, and film thickness 0.25 μM), flame ionization detector and TotalChrom Software. Hydrogen was the carrier gas. The amount of fatty acids was calculated using C23:0, EPA and DHA standards. The same response factor as DHA was assumed for the VLCPUFAs, as no standards are available.

The fatty acid compositions of Lipidmix 1 and 2 are shown in Table 1.

TABLE 1
Fatty acid composition of Lipidmix 1 and 2

| | Lipidmix 1 | Lipidmix 2 |
|---|---|---|
| | (mg/g) | (mg/g) |
| EPA | 24 | 28 |
| DPA | 45 | 39 |
| DHA | 151 | 191 |
| C24:4 | 5 | 0 |
| C24:5 | 68 | 1 |
| C24:6 | 33 | 1 |
| C26:3 | 2 | 0 |
| C26:4 | 7 | 0 |
| C26:5 | 20 | 0 |
| C26:6 | 62 | 0 |
| C26:7 | 9 | 0 |
| C28:4 | 2 | 0 |
| C28:5 | 10 | 0 |
| C28:6 | 14 | 0 |
| C28:7 | 4 | 0 |
| C28:8 | 163 | 0 |
| Total VLCPUFA | 323 | 2 |
| (C24-C28) | | |

### Test Diets Were Prepared With the Following Compositions

Test Diet 1: 10% fat (5% soybean oil, 5% lard), 17% protein, 5% fibre, 62% carbohydrates, minerals, vitamins (i.e. standard mice diet).

Test Diet 2: 10% fat (5% Lipidmix1 (incl. VLCPUFA), 5% lard), 17% protein, 5% fibre, 62% carbohydrates, minerals, vitamins (i.e. comprising VLCPUFAs).

Test Diet 3: 10% fat (5% Lipidmix2, 5% lard), 17% protein, 5% fibre, 62% carbohydrates, minerals, vitamins (i.e. without VLCPUFAs).

All test diets were stored at -20° C.

### Animals

Mice from the strain C57/bl6 from Charles River were used in the feeding study. The body weight was around 25 g. The animals were housed in cages with free access to food and water at room temperature.

### Eye Tissue

8 individuals from Test Diet group 1 and 9 individuals from Test Diet groups 2 and 3 were sacrificed 29-33 days after start of feeding study. The whole eye apples containing retinal tissue were carefully dissected from the animals by trained personnel. The samples were immediately frozen on dry ice and shipped to Nofima, Norway, for extraction and separation of phospholipid. The fatty acid analyses of prepared samples were done at Epax Norway. Total lipids were extracted from the mice eye tissues by the method by Folch et al.¹ Lipid classes were separated using thin layer chromatography (TLC). The phospholipid fractions were used for the fatty acid analyses.

### Blood Plasma

Blood samples were taken from 2 mice from each test diet groups sacrificed 33 days after start of feeding study. The samples were taken from aorta right after death. The samples were immediately frozen on dry ice and shipped to Epax Norway for analysis. 1 ml of a solution containing 0.05157 mg/ml C23:0 internal std was added to a test tube and the solvent was evaporated under a stream of nitrogen. The same test tube was then added the blood plasma and the weight of tissue noted. 3.5 ml of a solution containing 0.5M Sodium methoxide in methanol was added and the test tube was then heated in a boiling water bath for 1 hour. After cooling 5 ml of BCL3 was added and the test tube was heated in the boiling bath for 5 min. After heating the test tube was added 0.6 ml of isooctane and washed with 5 ml of saturated sodium chloride in water. The isooctane phase was transferred to micro-vials and injected directly on the GC.

### Fatty Acid Analyses of Eye Tissue Samples

The fatty acid analysis was done on a Perkin Elmer, Clarius 680/600T GC-MS using an Agilent CP Wax 52 B (CP7713) column. The peak area from chromatograms obtained from simultaneous single ions scans of 67, 79 and 91 m/z were used for quantification of the LC and VLCPUFA fatty acids. The response factor for DHA (relative to C23:0) using this setup was calculated by using standard solutions with known concentrations of DHA and C23:0. As no standards are available for the VLCPUFAs, the same response factor as for DHA was assumed, and used to calculate mg fatty acid/g tissue for the VLCPUFA.

### Results Eye

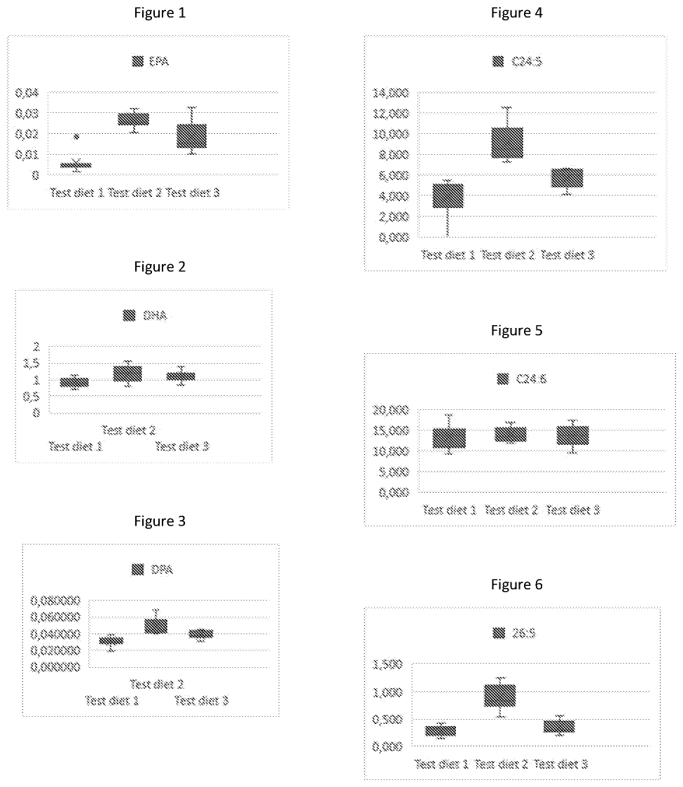

The results of the analysis of PUFAs with 22 carbons or more are shown in Table 2 below, and the results for each fatty acid are shown in FIGS. 1 to 8, wherein

FIG. 1. Content of EPA (mg/g tissue) in eye (apple) from mice fed Test Diet 1, 2 and 3.

FIG. 2. Content of DHA (mg/g tissue) in eye (apple) from mice fed Test Diet 1, 2 and 3.

FIG. 3. Content of DPAn3 (mg/g tissue) in eye (apple) from mice fed Test Diet 1, 2 and 3.

FIG. 4. Content of C24:5n3 (μg/g tissue) in eye (apple) from mice fed Test Diet 1, 2 and 3.

FIG. 5. Content of C24:6n3 (μg/g tissue) in eye (apple) from mice fed Test Diet 1, 2 and 3.

FIG. 6. Content of C26:5n3 (μg/g tissue) in eye (apple) from mice fed Test Diet 1, 2 and 3.

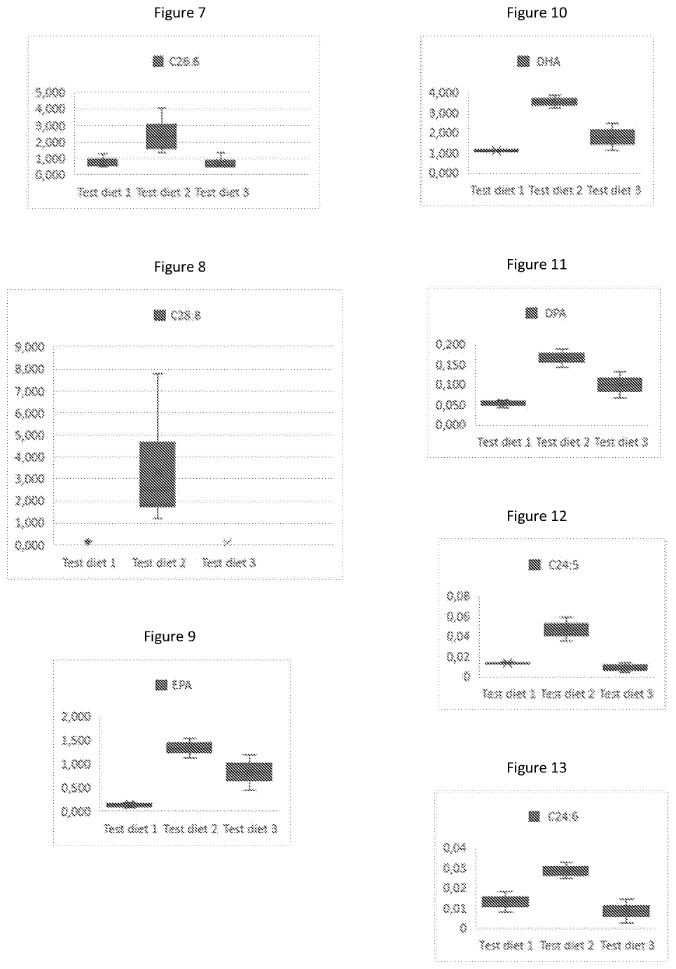

FIG. 7. Content of C26:6n3 (μg/g tissue) in eye (apple) from mice fed Test Diet 1, 2 and 3.

FIG. 8. Content of C28:8n3 (μg/g tissue) in eye (apple) from mice fed Test Diet 1, 2 and 3.

TABLE 2
Calculated amount of fatty acids in eye (apple) tissue of mice.

| | | Weight | Weight | EPA | DHA | DPA | C24:5 | C24:6 | C26:5 | C26:6 | C28:8 |
|---|---|---|---|---|---|---|---|---|---|---|---|
| | Test | tissue | intern std | mg/g | mg/mg | mg/g | μg/g | μg/g | μg/g | μg/g | μg/g |
| id | Diet No. | G | mg | tissue | tissue | tissue | tissue | tissue | tissue | tissue | tissue |
| P | 1 | 0.047 | 0.0974 | 0.002 | 0.940 | 0.031 | 2.439 | 9.259 | 0.150 | 0.463 | 0.000 |
| 27 | | | | | | | | | | | |
| P | 1 | 0.048 | 0.0974 | 0.004 | 1.024 | 0.035 | 5.120 | 15.671 | 0.207 | 0.538 | 0.000 |
| 28 | | | | | | | | | | | |
| P | 1 | 0.04 | 0.0974 | 0.004 | 0.697 | 0.019 | 5.465 | 14.686 | 0.357 | 0.956 | 0.000 |
| 29 | | | | | | | | | | | |
| P | 1 | 0.047 | 0.0974 | 0.006 | 0.797 | 0.028 | 4.660 | 14.076 | 0.355 | 0.993 | 0.000 |
| 30 | | | | | | | | | | | |
| P | 1 | 0.048 | 0.0974 | 0.004 | 0.843 | 0.031 | 4.359 | 13.524 | 0.331 | 0.852 | 0.000 |
| 31 | | | | | | | | | | | |
| P | 1 | 0.046 | 0.0974 | 0.018 | 0.813 | 0.029 | 4.112 | 10.327 | 0.322 | 0.543 | 0.142 |
| 32 | | | | | | | | | | | |
| P | 1 | 0.048 | 0.0974 | 0.003 | 1.122 | 0.039 | 4.979 | 12.865 | 0.214 | 0.614 | 0.000 |
| 33 | | | | | | | | | | | |
| P | 1 | 0.049 | 0.0974 | 0.005 | 1.023 | 0.035 | 0.000 | 18.822 | 0.422 | 1.258 | 0.000 |
| 35 | | | | | | | | | | | |
| P | 2 | 0.041 | 0.0974 | 0.025 | 1.381 | 0.058 | 12.525 | 13.322 | 0.767 | 2.802 | 3.513 |
| 36 | | | | | | | | | | | |
| P | 2 | 0.034 | 0.0974 | 0.033 | 1.240 | 0.043 | 7.302 | 13.675 | 1.164 | 1.756 | 2.151 |
| 37 | | | | | | | | | | | |
| P | 2 | 0.038 | 0.0974 | 0.022 | 1.512 | 0.045 | 7.873 | 12.274 | 0.845 | 1.902 | 2.338 |
| 38 | | | | | | | | | | | |
| P | 2 | 0.041 | 0.0974 | 0.021 | 1.573 | 0.069 | 8.162 | 12.776 | 0.542 | 1.372 | 1.311 |
| 39 | | | | | | | | | | | |
| P | 2 | 0.045 | 0.0974 | 0.024 | 0.805 | 0.040 | 9.526 | 15.352 | 1.260 | 2.442 | 4.482 |
| 41 | | | | | | | | | | | |
| P | 2 | 0.039 | 0.0974 | 0.025 | 0.827 | 0.044 | 7.646 | 11.977 | 0.786 | 3.316 | 4.801 |
| 42 | | | | | | | | | | | |
| P | 2 | 0.043 | 0.0974 | 0.027 | 0.983 | 0.040 | 8.521 | 16.164 | 0.713 | 1.444 | 1.180 |
| 43 | | | | | | | | | | | |
| P | 2 | 0.046 | 0.0974 | 0.030 | 1.205 | 0.051 | 10.453 | 16.906 | 0.869 | 2.312 | 2.473 |
| 44 | | | | | | | | | | | |
| P | 2 | 0.052 | 0.0974 | 0.135 | 1.085 | 0.057 | 10.656 | 14.029 | 1.054 | 4.054 | 7.796 |
| 45 | | | | | | | | | | | |
| P | 3 | 0.04 | 0.0974 | 0.033 | 0.891 | 0.036 | 5.979 | 17.465 | 0.305 | 0.505 | 0.000 |
| 16 | | | | | | | | | | | |
| P | 3 | 0.047 | 0.0974 | 0.015 | 1.405 | 0.045 | 4.103 | 12.308 | 0.284 | 0.591 | 0.000 |
| 17 | | | | | | | | | | | |
| P | 3 | 0.045 | 0.0974 | 0.019 | 0.851 | 0.031 | 6.571 | 15.349 | 0.569 | 1.363 | 0.000 |
| 18 | | | | | | | | | | | |
| P | 3 | 0.049 | 0.0974 | 0.024 | 1.209 | 0.046 | 6.641 | 16.223 | 0.473 | 0.473 | 0.000 |
| 19 | | | | | | | | | | | |
| P | 3 | 0.037 | 0.0974 | 0.026 | 1.212 | 0.043 | 5.993 | 11.142 | 0.453 | 0.734 | 0.248 |
| 20 | | | | | | | | | | | |
| P | 3 | 0.044 | 0.0974 | 0.013 | 1.226 | 0.041 | 4.921 | 12.944 | 0.236 | 0.533 | 0.000 |
| 21 | | | | | | | | | | | |
| P | 3 | 0.041 | 0.0974 | 0.016 | 1.091 | 0.038 | 6.097 | 15.716 | 0.294 | 0.647 | 0.000 |
| 22 | | | | | | | | | | | |
| P | 3 | 0.043 | 0.0974 | 0.012 | 1.026 | 0.037 | 6.552 | 13.684 | 0.312 | 0.581 | 0.000 |
| 23 | | | | | | | | | | | |
| P | 3 | 0.05 | 0.0974 | 0.010 | 1.001 | 0.037 | 4.744 | 9.479 | 0.203 | 0.511 | 0.000 |
| 40 | | | | | | | | | | | |

The results of the tissue analysis show slightly higher levels of EPA, DPA and DHA in eye tissue of mice fed with the Test Diets 2 and 3 compared to control (Test Diet 1). There seems to be no difference between Test Diet 2 and 3. These diets contain similar amounts of EPA, DPA and DHA.

The PL-extracts from mice fed Test Diet 2 (comprising VLCPUFAs) show higher levels of VLCPUFA than for the mice fed Test Diet 1 and 3. Especially for the VLCPUFAs C26:6 and C28:8 this is very clear.

### Results Plasma

The results of the analysis of PUFA fatty acids with 20 carbons or more found in blood plasma are shown in Table 3. The results for each fatty acid are shown in FIGS. 9 to 16, wherein

FIG. 9. Content of EPA (μg/g tissue) in blood plasma from mice fed Test Diet 1, 2 and 3.

FIG. 10. Content of DHA (μg/g tissue) in blood plasma from mice fed Test Diet 1, 2 and 3.

FIG. 11. Content of DPAn3 (μg/g tissue) in blood plasma from mice fed Test Diet 1, 2 and 3.

FIG. 12. Content of C24:5n3 (μg/g tissue) in blood plasma from mice fed Test Diet 1, 2 and 3.

FIG. 13. Content of C24:6n3 (μg/g tissue) in blood plasma from mice fed Test Diet 1, 2 and 3.

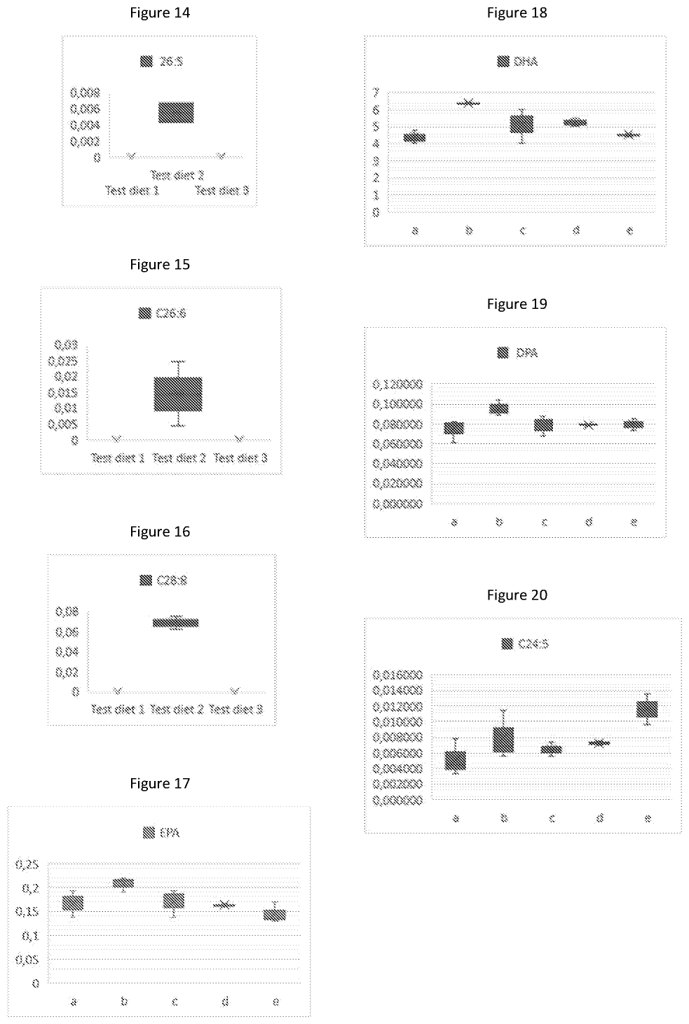

FIG. 14. Content of C26:5n3 (μg/g tissue) in blood plasma from mice fed Test Diet 1, 2 and 3.

FIG. 15. Content of C26:6n3 (μg/g tissue) in blood plasma from mice fed Test Diet 1, 2 and 3.

FIG. 16. Content of C28:8n3 (μg/g tissue) in blood plasma from mice fed Test Diet 1, 2 and 3.

TABLE 3
Calculated amount of fatty acids in blood plasma from mice.

| | | Weight | Weight | EPA | DHA | DPA | C24:5 | C24:6 | C26:5 | C26:6 | C28:8 |
|---|---|---|---|---|---|---|---|---|---|---|---|
| | Test | tissue | intern std | μg/g | μg/g | μg/g | μg/g | μg/g | μg/g | μg/g | μg/g |
| id | Diet No. | G | (mg) | tissue | tissue | tissue | tissue | tissue | tissue | tissue | tissue |
| 18 | 1 | 0 | 0.0974 | 0.071 | 1.039 | 0.041 | 0.014337 | 0.008194 | 0 | 0 | 0 |
| 19 | 1 | 0 | 0.0974 | 0.184 | 1.184 | 0.062 | 0.012311 | 0.018068 | 0 | 0 | 0 |
| 15 | 2 | 0 | 0.0974 | 1.125 | 3.843 | 0.143 | 0.03479 | 0.024453 | 0.004303 | 0.004303 | 0.062442 |
| 19 | 2 | 0 | 0.0974 | 1.542 | 3.197 | 0.189 | 0.058557 | 0.032546 | 0.006665 | 0.024539 | 0.075126 |
| 39 | 3 | 0 | 0.0974 | 0.443 | 1.088 | 0.066 | 0.014089 | 0.014275 | 0 | 0 | 0 |
| 40 | 3 | 0 | 0.0974 | 1.186 | 2.475 | 0.132 | 0.003638 | 0.002699 | 0 | 0 | 0 |

The results show that EPA, DHA and DPA levels are similar in all samples, with a trend that the group fed Test Diet 2 and 3 have higher levels than the control with standard mice feed (Test Diet 1). This is expected as Test Diet 2 and 3 comprise EPA, DHA and DPA, while the standard mice diet does not contain these fatty acids.

For the VLC fatty acids, there are significant higher levels found in blood plasma of the group fed Test Diet 2, which had VLC fatty acids in the feed. This is especially clear for the fatty acids C26:5, C26:6 and C28:8 where significant levels were found in the group which had Test Diet 2, while no detectable amounts were found in the two other groups.

### Conclusion

The feeding study in mice showed that orally administered VLC fatty acids were taken up by eye tissue. Eye tissue from mice with VLCPUFA in the diet had higher levels of VLCPUFA than controls.

Very long chain lipid components in eye tissue are known to play an important role for the retina and retinal functions. This example supports the invention that a composition of VLCFAs are taken up by tissue and can be used for treatment of eye diseases and in general for maintaining good eye health.

The feeding study in mice also showed that orally administered VLC fatty acids were taken up in blood plasma. Blood plasma from mice fed with a diet comprising VLCPUFAs had measurable and significant higher levels of VLC fatty acids than controls.

This example supports the invention that a composition of VLCFAs can be transported to blood plasma for further distribution in other tissues. Absorption and transport in organisms are important steps for the role of active compounds towards various diseases and in general for maintaining good health.

### Example 1A

### Supplementation With VLCPUFA in Mice—Effect on Fatty Acid Composition of Skin, Brain, Testis, Liver and Heart

### Lipid Compositions and Test Diets

The same lipid compositions, test diets and animals as described in Example 1 were used.

As provided in Example 1, Test Diet No. 2 comprises VLCPUFAs.

### Tissue Preparation

8 individuals from Test Diet group 1 and 9 individuals from Test Diet groups 2 and 3 were sacrificed 29-33 days after start of feeding study. Skin, brain, testis, liver and heart tissue samples were carefully dissected from 5 of the animals in each diet group by trained personnel. The samples were immediately frozen on dry ice and shipped to Nofima, Norway, for extraction and separation of phospholipid. The fatty acid analyses of prepared samples were done at Epax Norway.

Total lipids were extracted from the tissues by the method of Folch et al.¹ Lipid classes were separated using thin layer chromatography (TLC). The phospholipid (PL) fractions were used for the fatty acid analyses for all tissue samples, while also Triglyceride (TAG) fractions were analysed for liver and heart samples.

### Fatty Acid Analyses of Skin, Brain, Testis, Liver and Heart Tissue Samples

The fatty acid analysis was done on a Perkin Elmer, Clarius 680/600T GC-MS using an Agilent CP Wax 52 B (CP7713) column. The peak area from chromatograms obtained from simultaneous single ions scans of 67, 79 and 91 m/z were used for quantification of the LC and VLCPUFA fatty acids. The response factor for DHA (relative to C23:0) in this setup was calculated by using standard solutions with known concentrations of DHA and C23:0. As no standards are available for the VLCPUFAs, the same response factor as for DHA was assumed, and used to calculate mg fatty acid/g tissue for the VLCPUFA.

### Results Skin

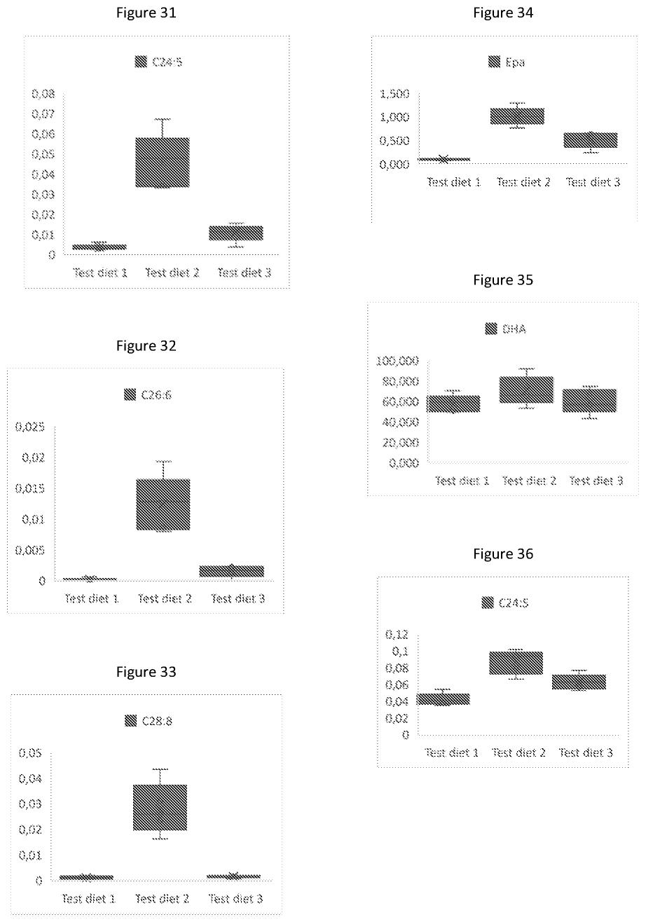

The results of the analysis of PUFAs with 22 carbons or more in skin tissue are shown in Table A1 below, and the results for each fatty acid are shown in FIGS. 31 to 33, wherein

FIG. 31. Content of C24:5n3 (mg/g tissue) in skin from mice fed Test Diet 1, 2 and 3.

FIG. 32. Content of C26:6n3 (mg/g tissue) in skin from mice fed Test Diet 1, 2 and 3.

FIG. 33. Content of C28:8n3 (mg/g tissue) in skin from mice fed Test Diet 1, 2 and 3.

TABLE A1
Average content of the different fatty acids in the PL
fractions of skin tissues from different diet groups

| | EPA | DPA | DHA | C24:5 | C24:6 | C26:6 | C26:7 | C28:8 |
|---|---|---|---|---|---|---|---|---|
| Test | mg/g | mg/g | mg/g | mg/g | mg/g | mg/g | mg/g | mg/g |
| Diet No. | tissue | tissue | tissue | tissue | tissue | tissue | tissue | tissue |
| 1 | 0.040 | 0.253 | 1.497 | 0.0036 | 0.0184 | 0.0001 | 0.0020 | 0.0011 |
| 2 | 0.356 | 0.428 | 2.268 | 0.0460 | 0.0514 | 0.0124 | 0.0033 | 0.0279 |
| 3 | 0.202 | 0.353 | 3.102 | 0.0106 | 0.0206 | 0.0015 | 0.0012 | 0.0016 |

The FIGS. 31-33 show the content of some major VLC fatty acids in skin tissue in mouse fed the different diets. Each box in the plot indicates the mean value +/− the standard deviation, brackets show highest and lowest value in each group.

### Results Brain

The results of the analysis of PUFAs with 22 carbons or more in brain tissue are shown in Table A2 below, and the results for each fatty acid are shown in FIGS. 34 to 37, wherein

FIG. 34. Content of EPA (mg/g tissue) in brain from mice fed Test Diet 1, 2 and 3.

FIG. 35. Content of DHA (mg/g tissue) in brain from mice fed Test Diet 1, 2 and 3.

FIG. 36. Content of C24:5n3 (mg/g tissue) in brain from mice fed Test Diet 1, 2 and 3.

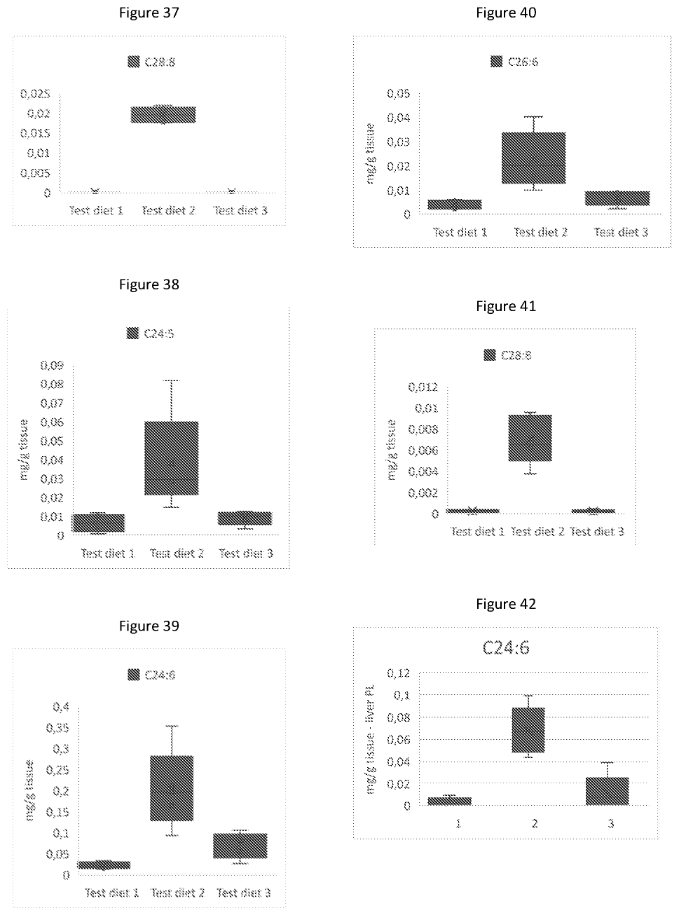

FIG. 37. Content of C28:8n3 (mg/g tissue) in brain from mice fed Test Diet 1, 2 and

TABLE A2
Average content of the different fatty acids in the PL
fractions of brain tissues from different diet groups

| | EPA | DPA | DHA | C24:5 | C24:6 | C26:6 | C28:8 |
|---|---|---|---|---|---|---|---|
| Test | mg/g | mg/g | mg/g | mg/g | mg/g | mg/g | mg/g |
| Diet No. | tissue | tissue | tissue | tissue | tissue | tissue | tissue |
| 1 | 0.0972 | 0.5990 | 57.6924 | 0.0420 | 0.2196 | 0.0067 | 0.0000 |
| 2 | 1.0084 | 1.5624 | 70.0784 | 0.0868 | 0.2582 | 0.0075 | 0.0198 |
| 3 | 0.5036 | 1.1824 | 59.7478 | 0.0628 | 0.2648 | 0.0032 | 0.0000 |

The FIGS. 34 to 37 show the content of the major VLC fatty acids in brain tissue in mouse fed the different diets. Each box in the plot indicates the mean value +/− one standard deviation, brackets show highest and lowest value in each group.

### Results Testis

The results of the analysis of PUFAs with 22 carbons or more in testis tissue are shown in Table A3 below, and the results for each fatty acid are shown in FIGS. 38 to 41, wherein

FIG. 38. Content of C24:5n3 (mg/g tissue) in testis from mice fed Test Diet 1, 2 and 3.

FIG. 39. Content of C24:6n3 (mg/g tissue) in testis from mice fed Test Diet 1, 2 and 3.

FIG. 40. Content of C26:6n3 (mg/g tissue) in testis from mice fed Test Diet 1, 2 and 3.

FIG. 41. Content of C28:8n3 (mg/g tissue) in testis from mice fed Test Diet 1, 2 and 3.

TABLE A3
Average content of the different fatty acids in PL fractions
of testis tissues from different diet groups.

| | EPA | DPA | DHA | C24:5 | C24:6 | C26:6 | C28:7 | C28:8 |
|---|---|---|---|---|---|---|---|---|
| Test | mg/g | mg/g | mg/g | mg/g | mg/g | mg/g | mg/g | mg/g |
| Diet No. | tissue | tissue | tissue | tissue | tissue | tissue | tissue | tissue |
| 1 | 0.025 | 0.061 | 2.63 | 0.0064 | 0.022 | 0.0043 | 0.0024 | 0.0002 |
| 2 | 0.477 | 0.213 | 9.73 | 0.0384 | 0.203 | 0.0225 | 0.0059 | 0.0070 |
| 3 | 0.100 | 0.095 | 4.72 | 0.0092 | 0.071 | 0.0070 | 0.0024 | 0.0002 |

The FIGS. 38-41 show the content of the major VLC fatty acids in testis tissue in mouse fed the different diets. Each box in the plot indicates the mean value +/− one standard deviation, brackets shows highest and lowest value in each group.

### Results Liver

### PL Fraction—Liver

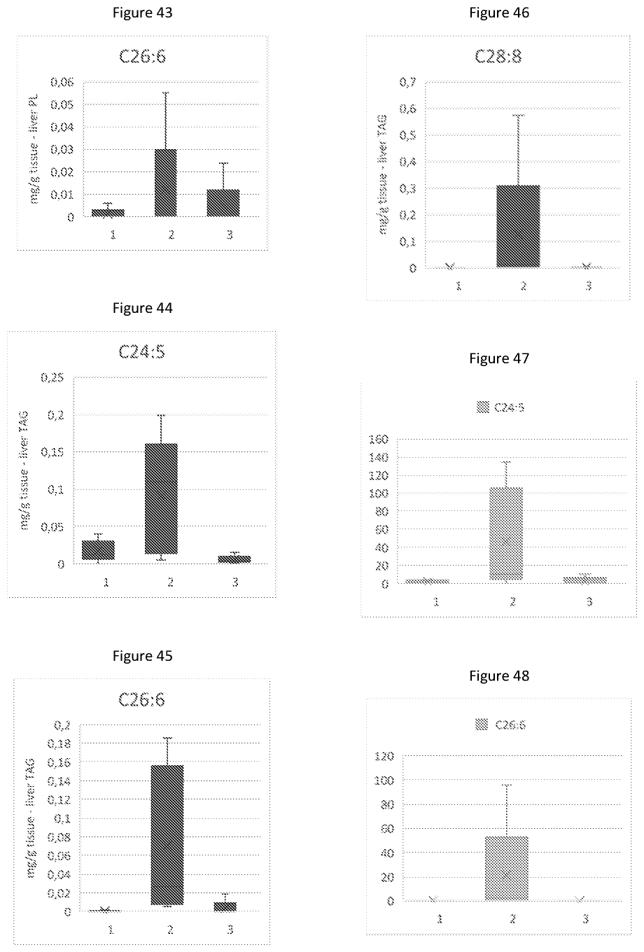

The results of the analysis of PUFAs with 22 carbons or more in PL-fraction of liver tissue are shown in Table A4 below, and the results for each fatty acid are shown in FIGS. 42 to 43, wherein

FIG. 42. Content of C24:6n3 (mg/g tissue) of PL in liver from mice fed Test Diet 1, 2 and 3.

FIG. 43. Content of C26:6n3 (mg/g tissue) of PL in liver from mice fed Test Diet 1, 2 and 3.

TABLE A4
average values of fatty acids in the PL-fraction of liver from each diet group

| | EPA | DPA | DHA | C24:5 | C24:6 | C26:4 | C26:5 | C26:6 | C26:7 | C28:8 |
|---|---|---|---|---|---|---|---|---|---|---|
| Test | mg/g | mg/g | mg/g | mg/g | mg/g | mg/g | mg/g | mg/g | mg/g | mg/g |
| Diet No. | tissue | tissue | tissue | tissue | tissue | tissue | tissue | tissue | tissue | tissue |
| 1 | 0.563 | 0.403 | 11.61 | 0.0000 | 0.0031 | 0.00000 | 0.00000 | 0.0012 | 0.0106 | 0.00000 |
| 2 | 4.550 | 1.625 | 44.13 | 0.0195 | 0.0678 | 0.00174 | 0.00248 | 0.0119 | 0.0159 | 0.00566 |
| 3 | 8.269 | 1.311 | 38.21 | 0.0047 | 0.0115 | 0.00859 | 0.00193 | 0.0047 | 0.0000 | 0.00652 |

The FIGS. 42-43 show the content of some major VLC fatty acids in the PL fraction of liver tissue in mouse fed the different diets. Each box in the plot indicates the mean value +/− one standard deviation, brackets show highest and lowest value in each group.

### TAG Fraction—Liver

The results of the analysis of PUFAs with 22 carbons or more in TAG-fraction of liver tissue are shown in Table A5 below, and the results for each fatty acid are shown in FIGS. 44 to 46, wherein

FIG. 44. Content of C24:5n3 (mg/g tissue) in TAG fraction of liver from mice fed Test Diet 1, 2 and 3.

FIG. 45. Content of C26:6n3 (mg/g tissue) in TAG fraction of liver from mice fed Test Diet 1,2 and 3.

FIG. 46. Content of C28:8n3 (mg/g tissue) in TAG fraction of liver from mice fed Test Diet 1,2 and 3.

TABLE A5
Average values of fatty acids in the TAG-fractions of liver tissue from each diet group

| | EPA | DPA | DHA | C24:4 | C24:5 | C24:6 | C26:4 | C26:5 | C26:6 | C26:7 | C28:8 |
|---|---|---|---|---|---|---|---|---|---|---|---|
| Test | mg/g | mg/g | mg/g | mg/g | mg/g | mg/g | mg/g | mg/g | mg/g | mg/g | mg/g |
| Diet No. | tissue | tissue | tissue | tissue | tissue | tissue | tissue | tissue | tissue | tissue | tissue |
| 1 | 0.219 | 0.328 | 1.874 | 0.00000 | 0.0174 | 0.057 | 0.0000 | 0.0000 | 0.0004 | 0.0015 | 0.0000 |
| 2 | 2.116 | 2.220 | 23.963 | 0.00845 | 0.0915 | 0.211 | 0.0129 | 0.0231 | 0.0708 | 0.0663 | 0.1245 |
| 3 | 2.667 | 2.049 | 14.549 | 0.00255 | 0.0058 | 0.028 | 0.0034 | 0.0000 | 0.0037 | 0.0000 | 0.0003 |

The FIGS. 44-46 show the content of some major VLC fatty acids in the TAG fraction of liver tissue in mouse fed the different diets. Each box in the plot indicates the mean value +/− one standard deviation, brackets show highest and lowest value in each group.

### Results Heart

### PL Fraction—Heart

The results of the analysis of PUFAs with 22 carbons or more from PL-fractions of hearts are shown in Table A6 below, and the results for each fatty acid are shown in FIGS. 47 to 48, wherein

FIG. 47. Content of C24:5n3 (μg/g tissue) in PL fraction of heart from mice fed Test Diet 1, 2 and 3.

FIG. 48. Content of C26:6n3 (μg/g tissue) in PL fraction of heart from mice fed Test Diet 1, 2 and 3.

TABLE A6
Average values of fatty acids in the PL-fractions of hearts from each diet group

| | EPA | DPA | DHA | C24:4 | C24:5 | C24:6 | C26:4 | C26:5 | C26:6 | C26:7 | C28:8 |
|---|---|---|---|---|---|---|---|---|---|---|---|
| Test | mg/g | mg/g | mg/g | μg/g | μg/g | μg/g | μg/g | μg/g | μg/g | μg/g | μg/g |
| Diet No. | tissue | tissue | tissue | tissue | tissue | tissue | tissue | tissue | tissue | tissue | tissue |
| 1 | 0.019 | 0.445 | 9.281 | 0.019 | 2.023 | 2.32 | 0.000 | 0.000 | 0.000 | 0.083 | 0.000 |
| 2 | 0.263 | 0.959 | 17.066 | 7.661 | 46.124 | 26.97 | 3.762 | 3.712 | 21.54 | 14.884 | 4.256 |
| 3 | 0.255 | 0.742 | 25.218 | 0.710 | 2.645 | 12.79 | 7.457 | 2.533 | 0.000 | 0.000 | 5.736 |

The FIGS. 47-48 show the content of some major VLC fatty acids in PL-fraction of heart tissue in mouse feed the different diets. Each box in the plot indicates the mean value +/− one standard deviation, brackets show highest and lowest value in each group

### TAG Fraction—Heart

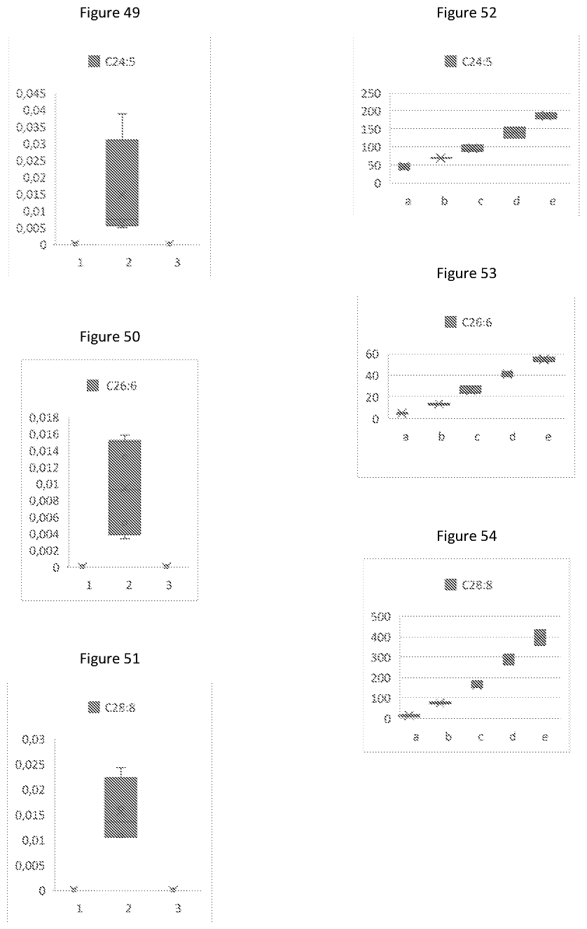

The results of the analysis of PUFAs with 22 carbons or more of TAG-fractions of heart tissues are shown in Table A7 below, and the results for each fatty acid are shown in FIGS. 49 to 51, wherein

FIG. 49. Content of C24:5n3 (mg/g tissue) in TAG fraction of heart from mice fed Test Diet 1,2 and 3.

FIG. 50 Content of C26:6n3 (mg/g tissue) in TAG fraction of heart from mice fed Test Diet 1,2 and 3.

FIG. 51. Content of C28:8n3 (mg/g tissue) in TAG fraction of heart from mice fed Test Diet 1,2 and 3.

TABLE A7
Average values of fatty acids in the TAG-fraction of heart tissue in each diet group

| | EPA | DPA | DHA | C24:4 | C24:5 | C24:6 | C26:4 | C26:5 | C26:6 | C26:7 | C28:8 |
|---|---|---|---|---|---|---|---|---|---|---|---|
| Test | mg/g | mg/g | mg/g | mg/g | mg/g | mg/g | mg/g | mg/g | mg/g | mg/g | mg/g |
| Diet No. | tissue | tissue | tissue | tissue | tissue | tissue | tissue | tissue | tissue | tissue | tissue |
| 1 | 0.0115 | 0.0000 | 0.0074 | 0.0000 | 0.0000 | 0.0000 | 0.0000 | 0.0000 | 0.0000 | 0.0000 | 0.0000 |
| 2 | 0.0165 | 0.0821 | 0.4557 | 0.0070 | 0.0147 | 0.0225 | 0.0095 | 0.0094 | 0.0094 | 0.0267 | 0.0155 |
| 3 | 0.0020 | 0.0362 | 0.1606 | 0.0000 | 0.0000 | 0.0000 | 0.0000 | 0.0000 | 0.0000 | 0.0000 | 0.0000 |

The FIGS. 49-51 show the content of some major VLC fatty acids in TAG fraction of heart tissue in mouse feed the different diets. Each box in the plot indicates the mean value +/− one standard deviation, brackets show highest and lowest value in each group.

### Conclusion

The feeding study in mice showed that orally administered VLC fatty acids were taken up by skin, brain, testis, liver and heart tissue. Tissue from mice with VLCPUFA in the diet had higher levels of VLCPUFA than controls. The fatty acids were generally taken up in both polar lipid fractions, including phospholipids, and neutral triglyceride lipid fractions, including triglycerides, of the tissues.

This example supports the invention that a composition of VLCFAs are taken up by tissue and can be used for treatment of diseases due to lack of VLCFA and in general for maintaining good function of these organs.

### Example 2

### Supplementation With VLCPUFA in Atlantic Salmon Feed—Effect on Fatty Acid Composition of Eye

### Lipid Composition

Lipidmix A was prepared from a standard anchovy fish oil. The crude fish oil was purified and ethylated, the ethylated oil was fractionated and up-concentrated by distillation, urea precipitation and Lithium-precipitation to obtain the desired composition. The VLCPUFA fraction was finally re-esterified to triglycerides by an enzymatic reaction with glycerol. Lipidmix A was on triglyceride form, containing small amounts mono- and di-glycerides. The fatty acid analysis of Lipidmix A was done on a Perkin Elmer, Clarius 500 with a split/splitless injector (splitless 1 min), using an Agilent CP Wax 52 B (CP7713) column, flame ionization detector and TotalChrom Software. Hydrogen was the carrier gas. The amount of fatty acids was calculated using the 23:0 internal standard. The response factor for DHA (relative to C23:0) was calculated by using standard solutions with known concentrations of EPA, DHA and C23:0. As no standards are available for the VLCPUFAs, the same response factor as for DHA was assumed, and used to calculate mg/g for the VLCPUFA. The results of the analysis of PUFA fatty acids with 20 carbons or more in Lipidmix A are shown in Table 4.

TABLE 4
Fatty acid composition of Lipidmix A

| Fatty acid | Lipidmix A (mg/g) |
|---|---|
| EPA | 103 |
| DPA | 54 |
| DHA | 197 |
| C24:4 | 5 |
| C24:5 | 23 |
| C24:6 | 12 |
| C26:3 | 1 |
| C26:4 | 6 |
| C26:5 | 15 |
| C26:6 | 19 |
| C26:7 | 3 |
| C28:4 | 3 |
| C28:5 | 4 |
| C28:6 | 7 |
| C28:7 | 1 |
| C28:8 | 76 |
| Total VLCPUFA | 175 |
| (C24-C28) | |

Lipidmix A comprised 175 mg/g VLCPUFA from fish oil and was used for preparing the test diets with different content of VLCPUFA.

### Test Diets

5 different test diets were prepared (a, b, c, d and e). The amount of ingredients was adjusted to ensure the same level in all test diets. Even the content of EPA and DHA was adjusted to the same concentration. The only difference was the content of VLCPUFA in the test diets. The adjustment of concentration of VLCPUFA in the test diets was done by adding various amount of Lipidmix A to the test diets.

The compositions of the different test diets are given in Table 5.

TABLE 5
Composition of Test diets (w %).

| Test diets: | a | B | c | d | e |
|---|---|---|---|---|---|
| Protein | 50.0 | 50.0 | 50.0 | 50.0 | 50.0 |
| Lipid | 20.0 | 20.0 | 20.0 | 20.0 | 20.0 |
| Starch | 5.9 | 5.9 | 5.9 | 5.9 | 5.9 |
| Ash | 13.9 | 13.9 | 13.9 | 13.9 | 13.9 |
| Water | 6.5 | 6.5 | 6.5 | 6.5 | 6.5 |
| Sum | 96.4 | 96.4 | 96.4 | 96.4 | 96.4 |
| Energy MJ/kg | 20.9 | 20.9 | 20.9 | 20.9 | 20.9 |
| EPA. % of test diet | 1.33 | 1.33 | 1.33 | 1.33 | 1.33 |
| DHA. % of test diet | 2.45 | 2.45 | 2.45 | 2.45 | 2.45 |
| Sum EPA + DHA. | 3.79 | 3.79 | 3.79 | 3.79 | 3.79 |
| % of test diet | | | | | |
| DPA. % of test diet | 0.62 | 0.69 | 0.75 | 0.82 | 0.89 |
| VLCPUFA. % of test | 0.00 | 0.35 | 0.71 | 1.06 | 1.41 |
| diet*) | | | | | |

### Feeding Experiment

Juvenile farmed Atlantic salmon (Salmo salar) with weight around 5 grams were used for the experiment.

5 different test diets (a-e, with 0.00 to 1.41 w% VLCPUFA of the diet) were prepared. 3 rearing tanks for each test diets (triplicate) were set up. 100 individual fishes were placed in each tank with recirculated fresh water. The feeding period was 4 weeks. At the end of the feeding experiment, 10 individual fishes from each tank were pooled, terminated, frozen on dry ice and stored at −40° C. before dissection of organs. The individual weight had increased to around 11 grams.

### Sample Preparation

The whole eye apple was dissected out of 10 individuals from each rearing tank, homogenized to make a pooled sample of 10 fish and frozen in liquid nitrogen and stored at −40° C. for later analyse of lipids. From each test diet there are triplicate samples (a pooled sample from three tanks).

Total lipids were extracted from the salmon eye tissues by the method by Folch et al. Lipid classes were separated using thin layer chromatography (TLC). The phospholipid fractions were used for the fatty acid analyses.

### Fatty Acid Analysis of Eye Tissue

The fatty acid analysis of extract from tissue was done on a Perkin Elmer, Clarius 680/600T GC-MS using an Agilent CP Wax 52 B (CP7713) column. The peak area from chromatograms obtained from simultaneous single ions scans of 67, 79 and 91 m/z were used for quantification of the LC- and VLCPUFAs. The response factor for DHA (relative to C23:0) using this setup was calculated by using standard solutions with known concentrations of DHA and C23:0. As no standards are available for the VLCPUFAs, the same response factor as for DHA was assumed, and used to calculate mg fatty acid/g tissue for the VLCPUFA.

### Results

The results of the analysis of PUFAs with 20 carbons or more in salmon eye tissue are shown in Table 6. The results for each fatty acid are shown in FIGS. 17 to 24, wherein

FIG. 17 provides the content of EPA (mg/g tissue) in eye apple tissue from Salmo salar fed Test diets a, b, c, d, e.

FIG. 18 provides the content of DHA (mg/g tissue) in eye apple tissue from Salmo salar fed Test diets a, b, c, d, e.

FIG. 19 provides the content of DPAn3 (mg/g tissue) in eye apple tissue from Salmo salar fed Test diets a, b, c, d, e.

FIG. 20 provides the content of C24:5n3 (mg/g tissue) in eye apple tissue from Salmo salar fed Test diets a, b, c, d, e.

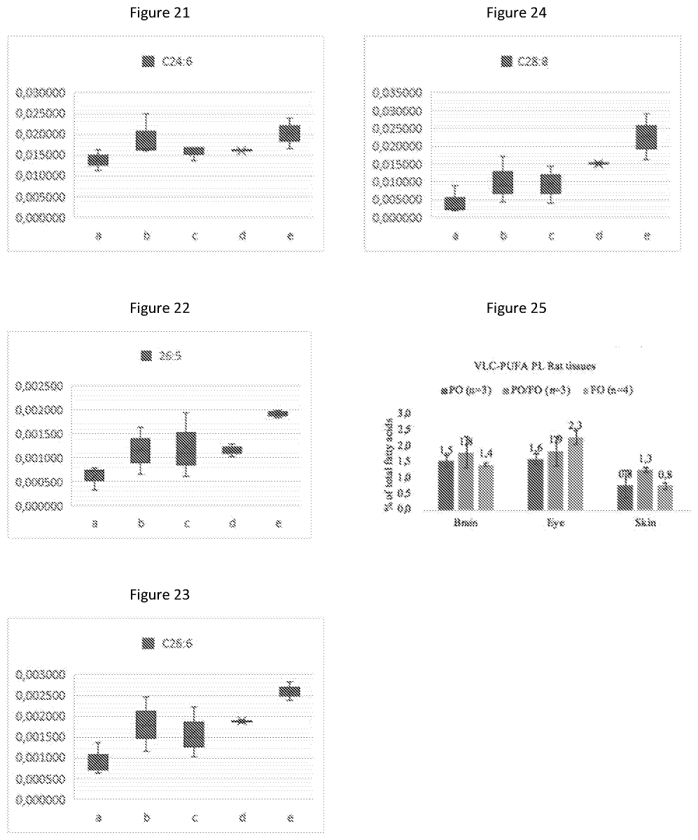

FIG. 21 provides the content of C24:6n3 (mg/g tissue) in eye apple tissue from Salmo salar fed Test diets a, b, c, d, e.

FIG. 22 provides the content of C26:5n3 (mg/g tissue) in eye apple tissue from Salmo salar fed Test diets a, b, c, d, e.

FIG. 23 provides the content of C26:6n3 (mg/g tissue) in eye apple tissue from Salom salar fed Test diets a, b, c, d, e.

FIG. 24 provides the content of C28:8n3 (mg/g tissue) in eye apple tissue from Salmo salar fed Test diets a, b, c, d, e.

TABLE 6
Calculated amount of PL-fraction fatty acids of eye apple tissues from
Salmo salar fed test diets with different concentration of VLCPUFA.

| | EPA | DHA | DPA | C24:5 | C24:6 | C26:5 | C26:6 | C28:8 |
|---|---|---|---|---|---|---|---|---|
| Test | mg/g | mg/g | mg/g | mg/g | mg/g | mg/g | mg/g | mg/g |
| diet | tissue | tissue | tissue | tissue | tissue | tissue | tissue | tissue |
| a | 0.13763 | 4.315834 | 0.061000 | 0.003333 | 0.011260 | 0.000325 | 0.000628 | 0.002460 |
| a | 0.171131 | 4.801838 | 0.082150 | 0.007794 | 0.016331 | 0.000712 | 0.001354 | 0.008707 |
| a | 0.192711 | 4.018847 | 0.079097 | 0.004416 | 0.013730 | 0.000778 | 0.000774 | 0.001810 |
| b | 0.190957 | 6.360014 | 0.088643 | 0.006845 | 0.016096 | 0.001145 | 0.001767 | 0.008895 |
| b | 0.219526 | 6.329992 | 0.094175 | 0.005517 | 0.016486 | 0.000650 | 0.001150 | 0.004373 |
| b | 0.213909 | 6.463981 | 0.104391 | 0.011491 | 0.024980 | 0.001643 | 0.002475 | 0.017011 |
| c | 0.19404 | 6.003039 | 0.088067 | 0.006363 | 0.016827 | 0.001082 | 0.001501 | 0.009269 |
| c | 0.137732 | 4.016379 | 0.067335 | 0.007390 | 0.013637 | 0.001952 | 0.002237 | 0.014293 |
| c | 0.177865 | 5.261819 | 0.079919 | 0.005513 | 0.016628 | 0.000611 | 0.001031 | 0.003940 |
| d | 0.160096 | 5.464854 | 0.078158 | 0.007018 | 0.015913 | 0.001021 | 0.001913 | 0.015528 |
| d* | 0.166931 | 2.385001 | 0.060147 | 0.003671 | 0.012256 | 0.000206 | 0.000413 | 0.000338 |
| d | 0.162362 | 5.032374 | 0.080003 | 0.007587 | 0.016190 | 0.001278 | 0.001845 | 0.014686 |
| e | 0.134477 | 4.627913 | 0.085352 | 0.013496 | 0.023810 | 0.001831 | 0.002372 | 0.016101 |
| e* | 0.131006 | 3.452695 | 0.051845 | 0.002238 | 0.008744 | 0.000458 | 0.000374 | 0.000497 |
| e | 0.169515 | 4.384236 | 0.073557 | 0.009686 | 0.016445 | 0.001997 | 0.002818 | 0.028940 |

*Outliers, excluded in further calculations and in graphs/figures.

The data shows that there is a trend with increasing content of VLCPUFA in the eye tissue (eye apple) of Salmo salar with increasing concentration of VLCPUFA in test diets. The effect is significant for C26:5, C26:6 and C28:8 with the test diets d and e which have the highest content of VLCPUFA—relative to the test diet without any VLCPUFA.

### Conclusion

The feeding study in salmon indicated that orally administered VLCPUFA resulted in increasing amount of some VLCPUFAs in eye tissue (eye apple). VLCPUFA are known to play an important role in human eye, and we have now shown that VLCPUFA is also part of the salmon fish eye. The eye retina is known to have a high expression of the ELOVL4 protein and a relatively high content of VLCPUFAs. Previous studies have indicated that the level of VLCPUFA in eye is determined solely by endogenous elongation and desaturation reactions. This study is the first to show that VLCPUFAS can be taken up from a dietary source.

This example supports the invention that a composition of VLCFAs can be used for supplementation and possible treatment and alleviation of eye related diseases or general eye health.

### Example 2B

### Supplementation With VLCPUFA in Atlantic Salmon Feed—Effect on Fatty Acid Composition of Skin, Brain, Heart and Liver

The same Lipid composition and Test diets as for Example 2 were used and the details of the Feeding experiment and the sample preparations are given in Example 2. The PL fractions were analysed for all tissues, while for the heart and liver tissues the TAG fractions were also analysed.

### Fatty Acid Analysis of Skin, Brain Heart and Liver Tissue

The fatty acid analysis of extract from tissue was done on a Perkin Elmer, Clarius 680/600T GC-MS using an Agilent CP Wax 52 B (CP7713) column. The peak area from chromatograms obtained from simultaneous single ions scans of 67, 79 and 91 m/z were used for quantification of the LC- and VLCPUFAs. The response factor for DHA (relative to C23:0) with this setup was calculated by using standard solutions with known concentrations of DHA and C23:0. As no standards are available for the VLCPUFAs, the same response factor as for DHA was assumed, and used to calculate mg fatty acid/g tissue for the VLCPUFA.

### Results Skin Tissue

The results of the analysis of PUFAs with 20 carbons or more in salmon skin tissue are shown in Table B1. The results for each fatty acid are shown in FIGS. 52 to 54, wherein

FIG. 52 provides the content of C24:5n3 (μg/g tissue) in skin tissue from Salmo salar fed Test diets a, b, c, d, e.

FIG. 53 provides the content of C26:6n3 (μg/g tissue) in skin tissue from Salom salar fed Test diets a, b, c, d, e.

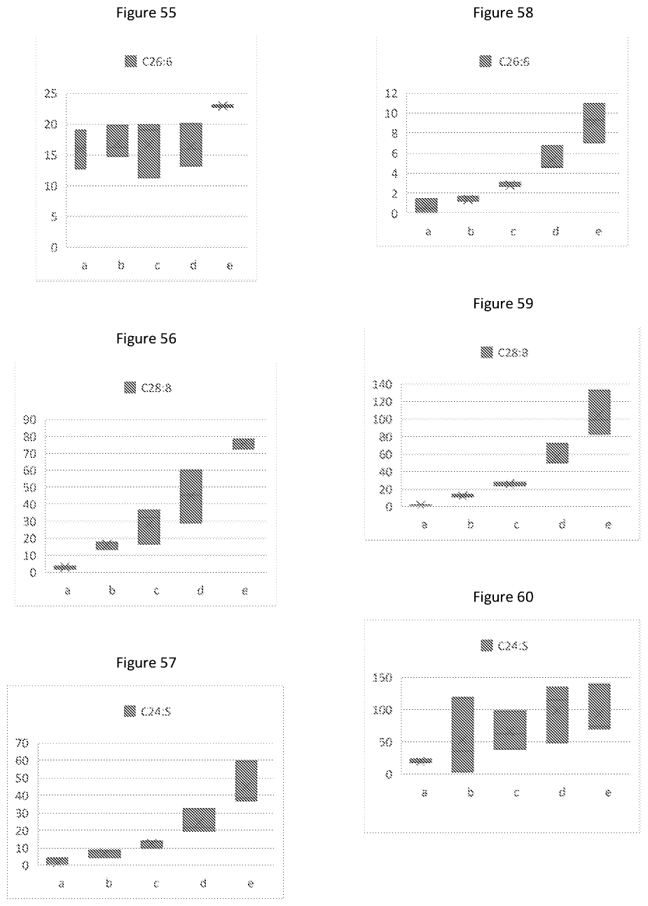

FIG. 54 provides the content of C28:8n3 (μg/g tissue) in skin tissue from Salmo salar fed Test diets a, b, c, d, e.

TABLE B1
Average values of different fatty acids from skin tissue from each diet group

| | EPA | DPA | DHA | C24:5 | C24:6 | C26:6 | C26:7 | C28:8 |
|---|---|---|---|---|---|---|---|---|
| Test | mg/g | mg/g | mg/g | μg/g | μg/g | μg/g | μg/g | μg/g |
| diet | tissue | tissue | tissue | tissue | tissue | tissue | tissue | tissue |
| a | 2.63 | 0.738 | 15.47 | 44.64 | 125.68 | 5.07 | 1.43 | 14.18 |
| b | 2.47 | 0.778 | 15.26 | 69.01 | 131.47 | 13.26 | 2.26 | 75.45 |
| c | 2.25 | 0.734 | 13.99 | 95.18 | 152.03 | 25.80 | 2.01 | 162.85 |
| d* | 2.51 | 0.866 | 15.42 | 139.70 | 181.52 | 40.87 | 3.40 | 288.98 |
| e* | 2.48 | 0.869 | 14.93 | 186.15 | 201.68 | 54.67 | 5.61 | 395.50 |

*one outlier removed

The FIGS. 52-54 show the content of some major VLC fatty acids in skin tissue in salmon fed the different diets. Each box in the plot indicates the mean value +/− the standard deviation, brackets show highest and lowest value in each group.

### Results Brain Tissue

The results of the analysis of PUFAs with 20 carbons or more in salmon brain tissue are shown in Table B2. The results for each fatty acid are shown in FIGS. 55 to 56, wherein

FIG. 55 provides the content of C26:6n3 (μg/g tissue) in brain tissue from Salmo salar fed Test diets a, b, c, d, e.

FIG. 56 provides the content of C28:8n3 (μg/g tissue) in brain tissue from Salmo salar fed Test diets a, b, c, d, e.

TABLE B2
Average values from each diet group of different fatty acids

| | EPA | DPA | DHA | C24:5 | C24:6 | C26:6 | C28:8 |
|---|---|---|---|---|---|---|---|
| Test | mg/g | mg/g | mg/g | μg/g | μg/g | μg/g | μg/g |
| diet | tissue | tissue | tissue | tissue | tissue | tissue | tissue |
| A | 5.73 | 2.38 | 31.53 | 148.87 | 474.91 | 15.91 | 2.83 |
| B | 5.57 | 2.29 | 34.26 | 155.25 | 451.63 | 16.87 | 16.14 |
| C | 4.69 | 1.93 | 25.92 | 137.70 | 405.50 | 16.71 | 28.39 |
| D | 3.94 | 1.78 | 22.51 | 123.94 | 330.87 | 16.35 | 44.77 |
| e* | 4.29 | 2.03 | 24.28 | 162.82 | 395.91 | 22.86 | 75.41 |

*one outlier removed

It is observed that the C28:8n3 fatty acid has been taken up considerably more than the other fatty acids.

The FIGS. 55-56 show the content of some major VLC fatty acids in brain tissue in salmon fed the different diets. Each box in the plot indicates the mean value +/− the standard deviation, brackets shows highest and lowest value in each group.

### Results Liver Tissue

### PL Fraction—Liver

The results of the analysis of PUFAs with 20 carbons or more in salmon liver PL tissue are shown in Table B3. The results for selected fatty acid are shown in FIGS. 57 to 59, wherein

FIG. 57 provides the content of C24:5n3 (μg/g tissue) in PL fraction of liver tissue from Salmo salar fed Test diets a, b, c, d, e.

FIG. 58 provides the content of C26:6n3 (μg/g tissue) in PL fraction of liver tissue from Salmo salar fed Test diets a, b, c, d, e.

FIG. 59 provides the content of C28:8n3 (μg/g tissue) in PL fraction of liver tissue from Salmo salar fed Test diets a, b, c, d, e.

TABLE B3
Average values of different fatty acids in the PL-fraction from each diet group

| | EPA | DPA | DHA | C24:5 | C24:6 | C26:4 | C26:5 | C26:6 | C26:7 | C28:5 | C28:6 | C28:8 |
|---|---|---|---|---|---|---|---|---|---|---|---|---|
| Test | mg/g | mg/g | mg/g | μg/g | μg/g | μg/g | μg/g | μg/g | μg/g | μg/g | μg/g | μg/g |
| diet | tissue | tissue | tissue | tissue | tissue | tissue | tissue | tissue | tissue | tissue | tissue | tissue |
| a | 0.39 | 0.11 | 3.54 | 1.44 | 12.38 | 0.75 | 1.04 | 0.64 | 0.00 | 0.00 | 0.00 | 0.75 |
| b | 0.28 | 0.09 | 2.99 | 6.88 | 22.35 | 0.32 | 0.66 | 1.37 | 0.39 | 0.77 | 0.52 | 12.28 |
| c | 0.25 | 0.09 | 2.92 | 12.38 | 25.15 | 0.57 | 1.70 | 2.80 | 0.74 | 0.55 | 1.22 | 26.17 |
| d | 0.27 | 0.11 | 3.22 | 24.48 | 33.80 | 1.54 | 4.66 | 5.31 | 1.37 | 3.84 | 2.09 | 57.51 |
| e | 0.27 | 0.13 | 3.14 | 44.90 | 48.19 | 3.14 | 9.36 | 9.08 | 2.22 | 4.55 | 4.40 | 104.84 |

It is observed that for Test Diet e) the fatty acids C24:5 and C26:6 were very clearly taken up in the polar phospholipid liver tissue, and this in a higher degree than in the TAG-fraction of the liver tissue, as provided below by the results in Table B4.

The FIGS. 57 to 59 show the content of some major VLC fatty acids in PL fraction of liver tissue in salmon fed the different diets. Each box in the plot indicates the mean value +/− the standard deviation, brackets shows highest and lowest value in each group.

### TAG Fraction—Liver

The results of the analysis of PUFAs with 20 carbons or more in TAG fractions of salmon liver tissues are shown in Table B4. The results for selected fatty acid are shown in FIGS. 60 to 62, wherein

FIG. 60 provides the content of C24:5n3 (μg/g tissue) in TAG fraction of liver tissue from Salmo salar fed Test diets a, b, c, d, e.

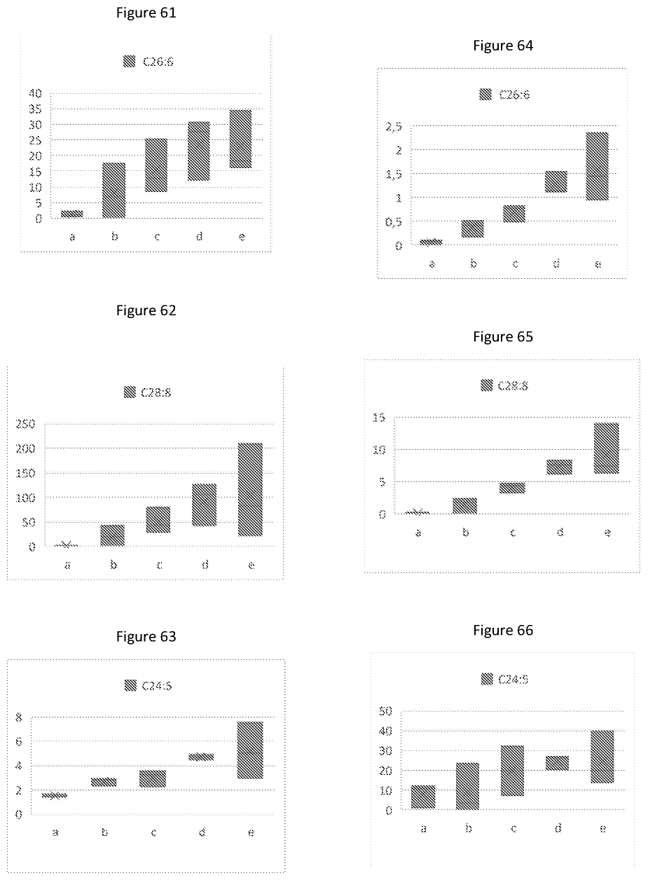

FIG. 61 provides the content of C26:6n3 (μg/g tissue) in TAG fraction of liver tissue from Salmo salar fed Test diets a, b, c, d, e.

FIG. 62 provides the content of C28:8n3 (μg/g tissue) in TAG fraction of liver tissue from Salmo salar fed Test diets a, b, c, d, e.

TABLE B4
Average values of fatty acids in the TAG-fraction from each diet group

| | EPA | DPA | DHA | C24:5 | C24:6 | C26:4 | C26:5 | C26:6 | C26:7 | C28:5 | C28:6 | C28:8 |
|---|---|---|---|---|---|---|---|---|---|---|---|---|
| Test | mg/g | mg/g | mg/g | μg/g | μg/g | μg/g | μg/g | μg/g | μg/g | μg/g | μg/g | μg/g |
| Diet, | tissue | tissue | tissue | tissue | tissue | tissue | tissue | tissue | tissue | tissue | tissue | tissue |
| a | 0.21 | 0.08 | 0.58 | 19.65 | 27.83 | 0.22 | 0.40 | 1.27 | 0.40 | 0.00 | 0.22 | 1.13 |
| b | 0.28 | 0.15 | 0.85 | 52.49 | 54.70 | 2.03 | 4.08 | 8.17 | 0.53 | 1.27 | 1.37 | 20.81 |
| c | 0.27 | 0.14 | 0.84 | 66.05 | 58.20 | 4.26 | 9.80 | 15.53 | 1.27 | 2.76 | 2.76 | 50.47 |
| d | 0.31 | 0.17 | 1.00 | 99.33 | 71.82 | 6.69 | 16.80 | 23.45 | 2.30 | 5.71 | 6.01 | 91.41 |
| e | 0.18 | 0.11 | 3.56 | 94.18 | 58.11 | 8.56 | 23.24 | 22.92 | 2.35 | 9.47 | 9.19 | 104.61 |

The FIGS. 60-62 show the content of some major VLC fatty acids in TAG fraction of liver tissue in salmon fed the different diets. Each box in the plot indicates the mean value +/− the standard deviation, brackets shows highest and lowest value in each group.

### Results Heart Tissue

### PL Fraction—Heart Tissue

The results of the analysis of PUFAs with 20 carbons or more in PL-tissues salmon heart tissues are shown in Table B5. The results for each fatty acid are shown in FIGS. 63 to 65, wherein

FIG. 63 provides the content of C24:5n3 (μg/g tissue) in PL fraction of heart tissue from Salmo salar fed Test diets a, b, c, d, e.

FIG. 64 provides the content of C26:6n3 (μg/g tissue) in PL fraction of heart tissue from Salmo salar fed Test diets a, b, c, d, e.

FIG. 65 provides the content of C28:8n3 (μg/g tissue) in PL fraction of heart tissue from Salmo salar fed Test diets a, b, c, d, e.

TABLE B5
Average values of fatty acids in the PL-fraction from each diet group

| | EPA | DPA | DHA | C24:5 | C24:6 | C26:4 | C26:5 | C26:6 | C26:7 | C28:5 | C28:6 | C28:7 | C28:8 |
|---|---|---|---|---|---|---|---|---|---|---|---|---|---|
| Test | mg/g | mg/g | mg/g | μg/g | μg/g | μg/g | μg/g | μg/g | μg/g | μg/g | μg/g | μg/g | μg/g |
| Diet | tissue | tissue | tissue | tissue | tissue | tissue | tissue | tissue | tissue | tissue | tissue | tissue | tissue |
| a | 0.0587 | 0.0181 | 0.385 | 1.5105 | 2.381 | 0.041 | 0.113 | 0.062 | 0.000 | 0.000 | 0.000 | 0.000 | 0.150 |
| b | 0.0568 | 0.0186 | 0.395 | 2.6873 | 2.760 | 0.047 | 0.171 | 0.368 | 0.162 | 0.069 | 0.068 | 0.041 | 1.396 |
| c | 0.0521 | 0.0181 | 0.358 | 3.0192 | 2.763 | 0.207 | 0.382 | 0.700 | 0.132 | 0.142 | 0.104 | 0.147 | 4.090 |
| d | 0.0554 | 0.0200 | 0.272 | 4.6969 | 3.668 | 0.316 | 0.798 | 1.268 | 0.259 | 0.476 | 0.390 | 0.112 | 7.325 |
| e | 0.0464 | 0.0178 | 0.305 | 5.1893 | 3.651 | 0.377 | 1.037 | 1.586 | 0.241 | 0.345 | 0.439 | 0.183 | 9.223 |

The FIGS. 63-65 show the content of some major VLC fatty acids in PL fraction of heart tissue in salmon fed the different diets. Each box in the plot indicates the mean value +/− the standard deviation, brackets shows highest and lowest value in each group.

### TAG Fraction—Heart Tissue

The results of the analysis of PUFAs with 20 carbons or more in TAG-fractions of salmon heart tissues are shown in Table B6. The results for each fatty acid are shown in FIGS. 66 to 68, wherein

FIG. 66 provides the content of C24:5n3 (μg/g tissue) in TAG fraction of heart tissue from Salmo salar fed Test diets a, b, c, d, e.

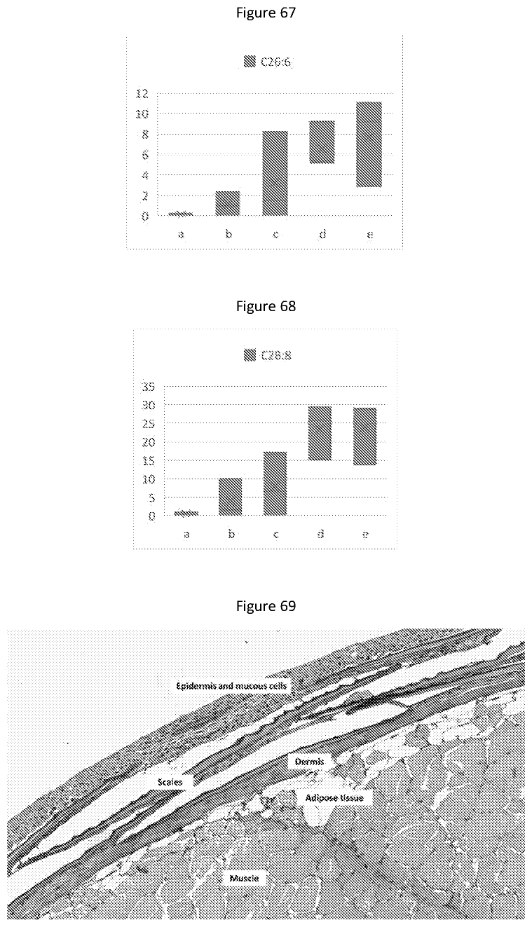

FIG. 67 provides the content of C26:6n3 (μg/g tissue) in TAG fraction of heart tissue from Salmo salar fed Test diets a, b, c, d, e.

FIG. 68 provides the content of C28:8n3 (μg/g tissue) in TAG fraction of heart tissue from Salmo salar fed Test diets a, b, c, d, e.

TABLE B6
Average values of fatty acids in the TAG-fraction of heart tissue from each diet group

| | EPA | DPA | DHA | C24:4 | C24:5 | C24:6 | C26:4 | C26:5 | C26:6 | C26:7 | C28:6 | C28:8 |
|---|---|---|---|---|---|---|---|---|---|---|---|---|
| Test | mg/g | mg/g | mg/g | μg/g | μg/g | μg/g | μg/g | μg/g | μg/g | μg/g | gg/g | μg/g |
| Diet | tissue | tissue | tissue | tissue | tissue | tissue | tissue | tissue | tissue | tissue | tissue | tissue |
| a | 0.142 | 0.059 | 0.390 | 2.63 | 8.04 | 11.35 | 0.000 | 0.048 | 0.087 | 0.443 | 0.124 | 0.368 |
| b | 0.050 | 0.024 | 0.154 | 6.90 | 8.97 | 1.57 | 0.109 | 0.423 | 1.122 | 1.827 | 0.496 | 3.878 |
| c | 0.096 | 0.045 | 0.270 | 18.79 | 19.63 | 0.00 | 0.498 | 1.638 | 3.238 | 0.988 | 0.764 | 7.929 |
| d | 0.111 | 0.057 | 0.312 | 28.68 | 24.48 | 0.00 | 1.574 | 3.909 | 6.584 | 3.537 | 1.718 | 23.433 |
| e | 0.098 | 0.050 | 0.272 | 21.29 | 24.41 | 3.20 | 1.473 | 4.344 | 7.693 | 2.381 | 1.784 | 23.815 |

The FIGS. 66-68 show the content of some major VLC fatty acids in TAG fraction of heart tissue in salmon fed the different diets. Each box in the plot indicates the mean value +/− the standard deviation, brackets shows highest and lowest value in each group.

### Conclusion

The feeding study in salmon indicated that orally administered VLCPUFA resulted in increasing amount of some VLCPUFAs in skin, brain, heart and liver, in addition to uptake in the eye as shown in Example 2.

The study shows that VLCPUFAS can be taken up from a dietary source and contribute to increased content in different tissues. It further shows that there are differences in the degree of uptake in the neutral lipid fractions and the polar fractions of the tissues.

### Example 3

### Content of VLCPUFA in Brain, Eye and Skin Tissues of Rat—Effect of Amount of Fish Oil in Feed

Sixteen male Zucker fa/fa rats (Crl:ZUC(Orl)-Lepr fa, (from Charles River Laboratories, Italy) were assigned to three experimental groups consisting of six rats with comparable mean body weight in each diet group. Rats were fed a diet with either plant oil, fish oil, or a 1:1 plant oil/fish oil mix (PO, FO or a 1:1 PO/FO mix) during a 4-week feeding intervention period. The organs skin, eyes and brain were dissected and stored at −80° C. for later analysis of VLCPUFAs. The fish oil contained 0.3-0.5% VLCPUFAs. The organ material was made available (from Nofima) for the MarOmega3-project owned by Pelagia/Epax.

### VLCPUFAs in Rat Tissues

Total lipids were extracted from the rat tissues (brain, eye and skin) following the method described by Folch et al¹. Six individual organ samples were analyzed per diet group. Main lipid classes were separated using thin layer chromatography (TLC). The phospholipid fractions were used for determination of VLCPUFA levels in the organs. The levels of VLCPUFAs identified in brain, eyes and skin tissue of rats in the different dietary groups are shown in FIG. 25, where VLCPUFA are in percentage of total fatty acids in brain, eye and skin PL of rats fed three different diets (PO, FO or a 1:1 PO/FO mix). The results are expressed as the mean with their SEM, where each value originates from 3-4 rats. Data were analyzed by a one-way ANOVA. There was no significance difference (P<0.05) between dietary groups within tissue, although there was a tendency to increased levels in the eyes with increased level of fish oil in the diet, in agreement with what was found in the salmon tissues.

### Conclusion

VLCPUFA were detected in all tissue samples. For rat eye there was a trend with increasing concentration of VLCPUFA with increasing levels of fish oil in the feed. In the fish oil there was only 0.3 to 0.5% VLCPUFAs. This example shows that the content of VLCPUFA in important tissues can be affected by the food intake. That means that a VLCFA-composition (concentrate) might be used for novel supplementation of VLCFAs to treat or alleviate diseases or help maintaining good health.

### Example 4

### Content of VLCPUFA in Brain, Eye and Skin Tissues of Atlantic Salmon—Effect of Amount of Fish Oil in Feed

### 1. Salmon Feeding Trial

The experimental fish were fed three dietary levels of two different fish oils (fish oil 1 and fish oil 2, both containing approximately 0.3-0.5% VLCPUFA) from a start fish weight of 100 gram to approximately doubling of weight. There were triplicate tanks per diet group. When the fish had reached 200 grams on the different diets, samples of brain, eye and skin were taken and frozen in liquid nitrogen and stored at −40° C. for later analyses of VLC-PUFA content in the organs. The purpose of the trial was to test how increasing dietary levels of fish oil influence the VLC-PUFA content in eyes, brain and skin of Atlantic salmon.

### VLCPUFA in Salmon Tissues

Total lipids were extracted from the salmon tissues by the method by Folch et al¹. A pooled sample of five organ samples per tank per tissue was used. Main lipid classes were separated using thin layer chromatography (TLC). The phospholipid (PL) fractions from the three organs were used for determination of VLCPUFA levels.

VLCPUFA methyl esters were analyzed on a Scion 436-GC with a split/splitless injector (splitless 1 min), using a Restek Rxi-5ms capillary column (length 30 m, internal diameter 0,25 mm, and film thickness 0.25 mM), flame ionization detector and TotalChrom Software. Hydrogen was the carrier gas.

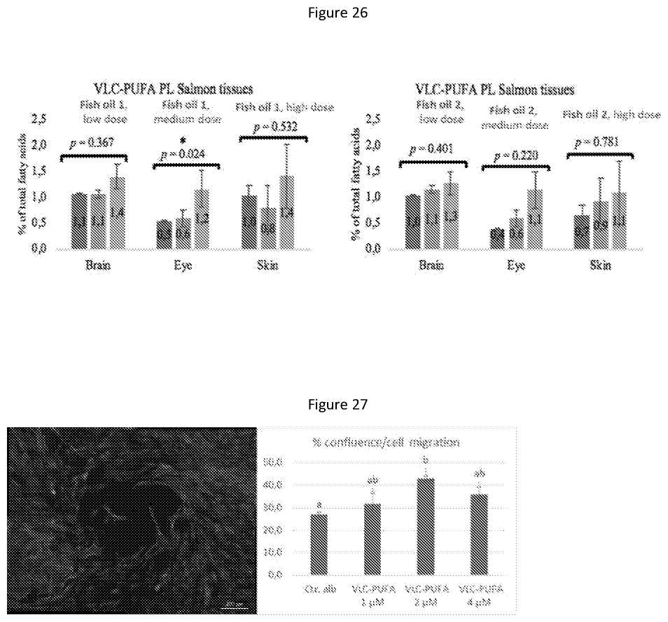

Detected levels (in percentage of total FAs) of VLC-PUFAs were significantly different in PL of eye tissue of fish fed increased dietary level of fish oil 1, as shown in FIG. 26. The FIG. 26 shows the identified VLC-PUFAs in the brain, eye and skin PL of Atlantic salmon fed three different levels of two fish oils (fish oil 1 and fish oil 2). The results are expressed as the mean with their SEM. Data were analyzed by a one-way ANOVA. The asterisks (*) indicate significant difference (P<0.05). However, although not significant, all organs showed tendencies to increased level of VLC-PUFA with higher doses of fish oil in the diets.

The fish oil had a low content of VLC-PUFA. Most probably one will need a higher concentration of VLC-PUFA in the feed in order to see significant effects.

### Conclusion

VLCPUFA showed a tendency to increase in all salmon tissues examined as fish oil levels increased in the diet. In eye tissue of fish, there was a significant difference. This example shows that the content of VLC-PUFA in important tissues can be affected by the food intake. That means that a VLCFA-composition (concentrate) might be used for novel supplementation of VLCFAs to treat or alleviate diseases or help maintaining good health.

### Example 5

### Effect of VLCPUFA on Skin Cells—For Wound Healing and Skin Health in General

The role of VLCPUFAs was examined in wound-healing models in-vitro. Human (1) and salmon skin cell (2) models were used, and the synthetic C26:6n-3 and a VLCPUFA concentrate from fish oil were tested.

### Lipid Compositions

Lipid composition A. VLCPUFA concentrate from fish oil

Lipid composition B: C26:6n-3: Pure synthetic fatty acid purchased from BOC Sciences (NY, USA)

Lipid composition A was prepared from a standard anchovy fish oil. The crude fish oil was purified and ethylated, the ethylated oil was fractionated and up-concentrated by distillation, urea precipitation and Lithium-precipitation to obtain the desired composition. The fractions were finally re-esterified to triglycerides by an enzymatic reaction with glycerol.

VLCPUFA methyl esters were analyzed on a Scion 436-GC with a split/splitless injector (splitless 1 min), using a Restek Rxi-5ms capillary column (length 30 m, internal diameter 0,25 mm, and film thickness 0.25 μM), flame ionization detector and TotalChrom Software. Hydrogen was the carrier gas.

The results of the analysis of PUFAs with 20 carbons or more are shown Table 7.

TABLE 7
Fatty acid composition of Lipid composition A and B

| | Lipid composition A | Lipid composition B |
|---|---|---|
| | Areal % | Areal % |
| EPA | 0 | |
| DPA | 0.2 | |
| DHA | 0.68 | |
| C24:1 | 1.29 | |
| C24:4 | 0.33 | |
| C24:5 | 1.47 | |
| C24:6 | 0.47 | |
| C26:1 | 2.97 | |
| C26:5 | 4.34 | |
| C26:6 | 9.56 | 100 |
| C26:7 | 0.95 | |
| C28:5 | 2.4 | |
| C28:6 | 5.31 | |
| C28:7 | 1.44 | |
| C28:8 | 62.25 | |
| C30:5 | 1.42 | |
| C30:6 | 2.74 | |
| C30:8 | 0.71 | |
| C32:8 | 0.21 | |
| Total VLC fatty acids | 96.57 | 100 |
| (C24-C32) | | |

### 1. In-Vitro Study in Cell Culture of Human Dermal Fibroblasts

A commercial human dermal fibroblast cell line (ATCC PCS-201-012) was cultured in Dulbecco's modified Eagle's medium according to the method described by Vuong et al².

### 1A. Cell Culture Study With Lipid Composition A

ATCC cells were seeded in wells with 2 mL culture media supplemented with 1, 2 and 4 μM Lipid composition A. Control was albumin in PBS. At ˜90-100% confluency, a scratch was created, and wells were thereafter photographed at several time points up to 24 hours. The migration of cells into the scratch/closure of wound over time was examined in light microscopy and images were taken. The scratch/wound closure rate was measured by % confluency in scratch opening (the higher values the better closure of wound). 2 μM of Lipid composition A resulted in significant higher wound closure rate by % confluency after 24 hours compared to the control (FIG. 27). The data shows that there is a significant better “wound healing” at 2 μM Lipid composition A relative to control.

FIG. 27 provides a fluorescence image of ATCC human fibroblasts supplemented with 4 μM Lipid composition A in culture media. The right panel of the figure shows the percentage of confluence in the scratch in the different concentration groups.

### 1B. Cell Culture Study With Lipid Composition B

ATCC cells were cultivated in a media supplemented with 10 and 20 μM Lipid composition B for 4 days before harvesting for determination of fatty acid composition. It was made a pooled sample of three replicates per group prior to lipid extraction by the method by Folch et al¹.

The content of C26:6n-3 in ATCC human skin cells was affected by adding Lipid composition B to the culture medium. The results showed a significant increase in C26:6n-3 from 0.7 to 5.5% of total FAs (p=0.001(ANOVA)).

### Proliferation Assay

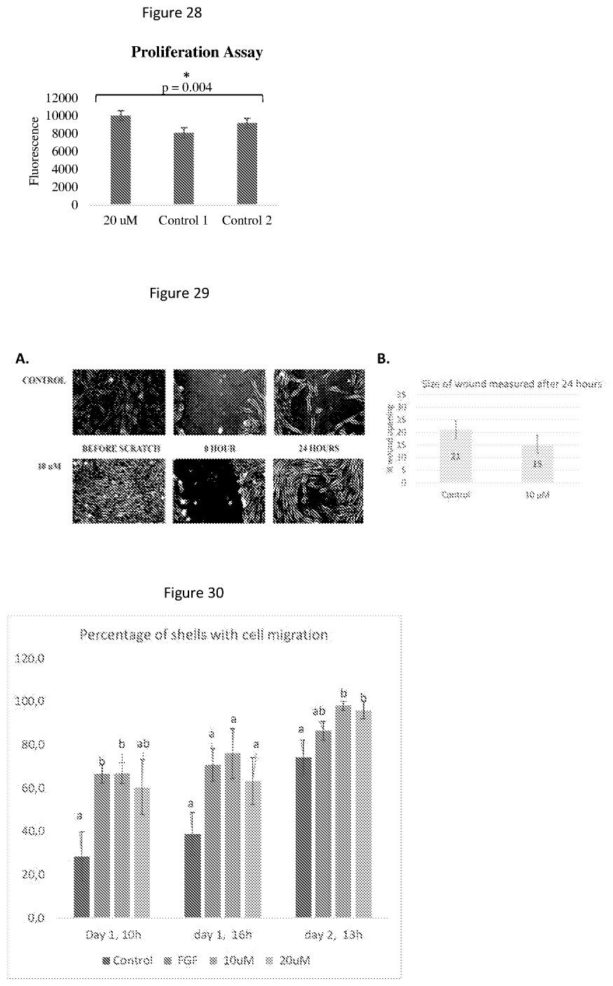

The proliferation assay measures the density/number of cells in culture by fluorescence staining of nucleic acids. The results show that ATCC cells cultivated in a media supplemented with Lipid composition B had a significantly higher cell count compared to controls (FIG. 28).

FIG. 28 provides the measurement of cell proliferation after incubation with Lipid composition B until about 50% confluency. Results are presented as the mean ±SEM (n=4). The asterisks (*) indicate significant difference between groups. Control 1 and 2 represent albumin equivalent to the albumin concentration in the 20 μM Lipid composition B substrate.

### Scratch Assay (In-Vitro Wound Healing Model)

Cells were seeded in wells with 2 mL culture media supplemented with 10 μM Lipid composition B (6 replicates). Control was albumin in PBS. At ˜90-100% confluency, a scratch was created, and wells were thereafter photographed at several time points up to 24 hours. The migration of cells into the scratch/closure of wound over time was examined in light microscopy and images were taken (FIG. 29).

FIG. 29 provides the effect of Lipid composition B on closure rate of scratch. Human ATCC dermal fibroblasts were incubated with Lipid composition B (10 μM) or (control, albumin in PBS) for 24 hours until monolayer was confluent. Cells were then scratched and cell migration into wound was followed at different time points (0 and 24 hours shown), using Fiji/ImageJ software, as illustrated in figure A. Scratch size was analyzed as mean percentage decline (calculated from each well's original size at 0 hours to size measured at 24 hours (n=6, results presented in figure B).

10 μM of the Lipid composition B group showed tendencies to increased wound closure rate by reduced size of wound diameter after 24 hours compared to control.

### 2. In Vitro-Study in Primary Cell Culture With Skin Cells From Atlantic Salmon

Primary cell cultures of skin cells (keratocytes) from salmon shells were isolated from freshwater Atlantic salmon. The shells were carefully placed in plate wells and incubated at 13° C. in growth media (L-15) supplemented with 10 μM or 20 μM of the Lipid composition B (26:6n-3) or 25 ng/mL fibroblast growth factor (FGF) as a positive control. Negative control was albumin in PBS.

Lipid composition B supplementation to culture media resulted in an increase in cellular content of the C26:6n-3 fatty acid from 0% in the control group to 1.4% in the 20 μM Lipid composition B group.

### Analyses of Cell Migration From Salmon Shells

The second day after isolation of shells, all wells with the different treatments were inspected under microscope and images taken.

FIG. 30 shows the results from the cell migration from salmon shells. Salmon shells were plucked from freshwater salmon and placed in wells with culture medium, incubated at 13° C. without CO2 and inspected for cell migration the following days. Treatments were 25 ng/mL FGF (n=7), 10 μM Lipid composition B (n=5), 20 μM Lipid composition B (n=5). Control was albumin in PBS. (n=6). The scale of the y-axis represents no cell migration (0%) to cell migration from all shells (100%). Different letters denote significant differences (p≤1.05).

At the first time point, there was a significant difference between the groups, showing that 1 μM Lipid composition B had a similar, immediate increased cell migration effect as the FGF (realtive to albumin control). At the second timepoint, the difference was not significant (P=0.061), however the control still clearly show less cell migration compared to the other groups. At the last time point there was a significant difference between the groups, and again the low dose (10 μM) Lipid composition B had the most effect on cell migration.

### Conclusion

The Lipid composition A (VLCPUFA-concentrate from fish oil) significantly increased cell migration in human fibroblast cells at 2 μM relative to control.

The Lipid composition B (synthetic C26:6n-3) significantly increased cell migration in salmon skin cell culture at 10 μM relative to control. The same trend was shown with Lipid composition B on human fibroblast cells.

This example shows the novel effect of VLCPUFAs on two different skin cell models and illuminates the immediate effect of VLCPUFA supplementation on skin cells for both human and fish. It indicates health beneficiary effects of these fatty acids in wound healing.

Ceramide is the main component of the stratum corneum of the epidermis layer of human skin. Very long chain lipid components are known to be linked to the ceramides. This example supports the invention in that a composition of VLCFAs can be used for wound-healing, inflammatory skin conditions and various other skin related diseases for both humans and animals/fish.

### Example 6

### Supplementation With VLCPUFA in Atlantic Salmon Feed—Evaluation of Skin From Juvenile Atlantic Salmon Fed Different Levels of VLCPUFA

To evaluate how different levels of VLCPUFAs in the feed affected skin and scale development in juvenile Atlantic salmon, fish fed either no, intermediate or high dietary levels of a VLCPUFA concentrate were analysed. The VLCPUFA concentrate called Lipidmix A, as described in Example 2, was included in fish feed in three different concentrations, and fed the three groups of fish;

Test diet a: 0% VLCPUFA,

Test diet c: 0.71% VLCPUFAs, and

Test diet e: 1.41% VLCPUFAs.

To capture changes involved in recruitment of mesenchymal stem cells, mineralization of scales and maturation of skin, small fish, just starting to develop scales were used. FIG. 69 shows the microanatomy of skin from Atlantic salmon showing the different layers, including epidermis with mucous cells, scales, dermis and the underlying adipose tissue and muscle.

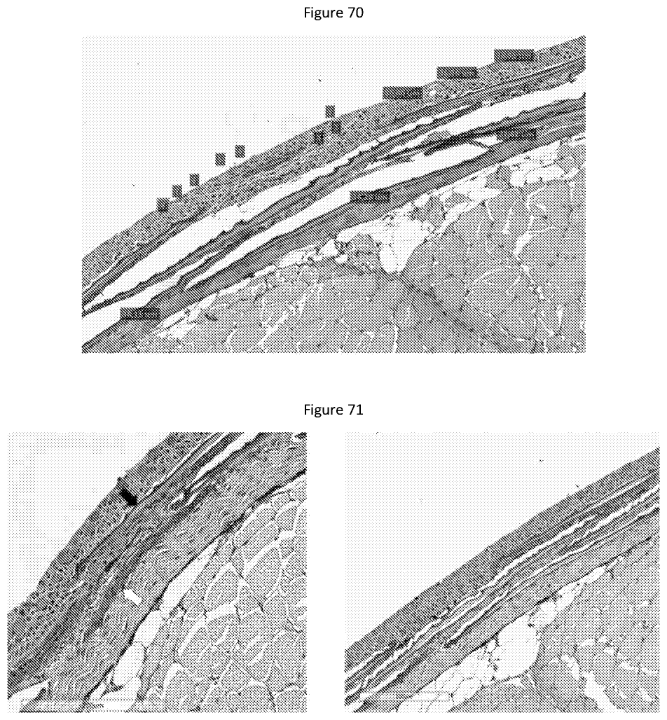

Skin from the three different groups of fish having been feed with different concentrations of VLCPUFAs were embedded in paraffin, sectioned and stained with histological stain for visualization of cellular structures and mucous cells (AB/PAS, FIG. 70). Sections were analyzed with microscopy, 40× using a Leica scanner and the ImageScope software. 15 fish from each group were used for these analyses. FIG. 70 shows the measures done in this trial included counting of mucous cells, thickness of the epidermis and dermis, as well as evaluation of scale development.

Results showed that fish fed 0% VLCPUFA (Test diet a) had less developed scales compared to fish from the fish fed intermediate (Test diet c) and high levels (Test diet e) of VLCPUFAs. Fish from the Test diet a-group also had thinner epidermal thickness. This indicated a less mature structure of the skin. Two different timepoints were evaluated. In FIG. 71, the development over time in fish from the Test diet a-group is illustrated, showing more mature scales at the final sampling. First sampling when the fish was 9.5 g (left picture) showing mineralized scale (black arrow) and developing scale (white arrow) and final sampling when the fish was 12 g (right picture).

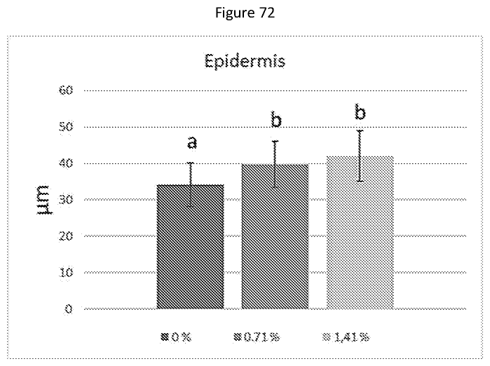

Measuring the epidermal thickness showed that fish fed higher doses of VLC-PUFA had thicker epidermis compared to fish fed the lower doses (FIG. 72). This may be an indication of more mature skin, as seen in other experiments with salmon, and may be because of more intense scale development. As shown by FIG. 72, the epidermis, as measured, significantly increased in thickness when salmon juveniles received a feed added VLCPUFA. The solid bars show the measured thickness of the epidermis in pm for test diet group a, c and e. The feed for these diet groups contained 0%, 0.71% and 1.41% by weight, respectively, of VCLPUFA. 15 skin samples were analyzed for each group of salmon. Significant changes are marked with different letters, P<0.05.

Samples are to be further analysed to evaluate the degree of mineralization and recruitment of mesenchymal stem cells. Preliminary results indicate that VLCPUFA-fed salmon have better scale development and more mature epidermis overall compared to fish without VLC-PUFA in the diet.

### Conclusion

The feeding study in salmon showed in vivo effects on salmon skin of supporting fish feed with VLCPUFAs. The results show that VLCPUFAs in the fish feed promotes skin with a thicker epidermis, improved scale development and more mature structure of the skin, indicating healthier skin in fish fed VLCPUFAs. The study shows that VLCPUFAs in diet positively effect skin development in salmon. This example supports the invention that a composition of VLCPUFAs can be used for supplementation and possible treatment and alleviation of skin diseases or general skin health.

### Example 7

### Supplementation With VLCFA in Mice—Effect on Fatty Acid Composition of Skin and Blood Plasma

### Lipid Compositions

A VLCFA concentrate (see Table 8 below) was prepared from a standard anchovy fish oil. The crude fish oil was purified and ethylated, the ethylated oil was fractionated and up-concentrated by distillation, to obtain the desired composition. The fractions were finally re-esterified to triglycerides by an enzymatic reaction with glycerol.

TABLE 8
Composition of VLCFA lipid mix used
for preparing test diet 4 and 5

| Fatty acid | A % | Mg/g |
|---|---|---|
| C20:5 n3 (EPA) | 0.24 | 2 |
| C22:0 | 0.82 | |
| C22:1 | 3.01 | |
| C22:5 n6 | 0.82 | |
| C22:5 n3 | 9.88 | |
| C22:6 n3 (DHA) | 49.53 | 452 |
| C24:0 | 0.11 | |
| C24:1 | 15.28 | 133 |
| C24:4n3 | 0.74 | |
| C24:5n3 | 1.31 | 12 |
| C24:6n3 | 1.41 | 13 |
| C26:1 | 1.29 | 11 |
| C26:4n3 | 0.50 | 4 |
| C26:5n3 | 0.47 | 4 |
| C26:6n3 | 0.95 | 8 |
| C26:7n3 | 0.30 | 3 |
| C28:5n3 | 0.11 | 1 |
| C28:6n3 | 0.16 | 1 |
| C28:7n3 | 0.22 | 2 |
| C28:8n3 | 3.22 | 28 |
| Sum VLCPUFA | 9.39 | 76 |
| Sum VLCMUFA | 16.57 | 143 |

TABLE 9
Composition of oils used for preparing test diets

| | EPA | DHA | VLCMUFA | VLCPUFA |
|---|---|---|---|---|
| Oil | a % | a % | a % | a % |
| Epax 3000TG | 19.96 | 12.45 | 0.25 | 0.41 |
| Epax 0460 TGN | 8.6 | 65.3 | 0.38 | 0.61 |
| Soy oil | 0 | 0 | 0 | 0 |

Five different diets were prepared by mixing either the VLCFA concentrate described (Test diet 4 and 5) above or two different fish oils produced by Epax Norway AS (EPAX 3000 TG and EPAX 0460 TGN) with soya oil. The mice were fed (by gavage) 100 mg/day of the different fatty acid mixes. The dose of the different fatty acids per mouse per day in the different diet groups are given in the Table 10 below.

TABLE 10
Composition of different test diets

| | | Dose | Dose | Dose |
|---|---|---|---|---|
| Diet | | EPA + DHA | VLCMUFA | VLCPUFA |
| Group | Oil | (mg/day) | (mg/day) | (mg/day) |
| 1 | Soya | 0 | 0 | 0 |
| 2 | EPAX 3000 TG | 5.4 | 0.04 | 0.07 |
| 3 | EPAX 0460 TGN | 8.3 | 0.03 | 0.07 |
| 4 | Low dose VLC | 4.2 | 1.3 | 0.7 |
| 5 | High dose VLC | 8.4 | 2.6 | 1.4 |

All test diets were stored at 0° C.

### Animals

Mice from the strain C57/bl6 from Charles River were used in the feeding study. The animals were housed in cages with free access to normal mice feed and water at room temperature.

### Fatty Acid Analysis

The fatty acid compositions of VLCFA concentrates, tissue extracts and blood plasmas were analysed on a Scion 436-GC with a split/splitless injector (splitless 1 min), using a Restek Rxi-5ms capillary column (length 30 m, internal diameter 0.25 mm, and film thickness 0.25 μM), flame ionization detector and Compass CDS Software. Hydrogen was the carrier gas. The amount of fatty acids was calculated using C23:0, EPA and DHA standards. The same response factor as DHA was assumed for the VLCPUFAs, as no standards are available. The VLC MUFAs were assumed to have same response factor as C23:0.

### Tissue Preparation

8 individuals from each Test Diets were sacrificed 4 weeks after start of feeding study. The different tissues were carefully dissected from the animals by trained personnel. The samples were immediately frozen on dry ice and shipped to Nofima, Norway, for extraction and separation of lipids classes. The fatty acid analyses of prepared samples were done at Epax Norway.

Total lipids were extracted from the mice tissues by the method by Folch et al.¹ Lipid classes were separated using thin layer chromatography (TLC). Total extract and Neutral lipid fractions were used for the fatty acid analyses.

Plasma samples were sent direct to Epax Norway and prepared for analysis as described in Example 1.

### Results Total Lipids—Skin Tissue

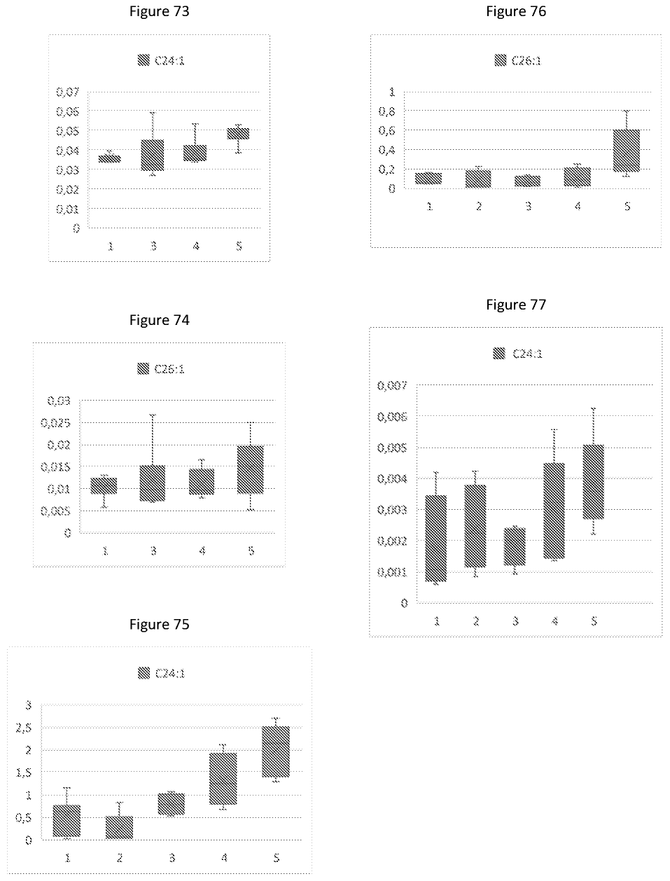

The results of the analysis of PUFAs with 22 carbons or more are shown in Table 11 below, and the results for selected fatty acids are shown in FIGS. 73 to 74, wherein

FIG. 73. Content of C24:1 (mg/g tissue) in skin from mice fed Test Diet 1, 2 and 3.

FIG. 74. Content of C26:1 (mg/g tissue) in skin from mice fed Test Diet 1, 2 and 3.

TABLE 11
Average values of different fatty acids in total lipid fraction from each diet group

| | EPA | DPA | DHA | C24:0 | C24:1 | C26:1 | C26:7 | C28:8 |
|---|---|---|---|---|---|---|---|---|
| Test | mg/g | mg/g | mg/g | mg/g | mg/g | mg/g | mg/g | mg/g |
| Diet No. | Tissue | Tissue | Tissue | Tissue | Tissue | Tissue | Tissue | Tissue |
| 1 | 0.966 | 0.115 | 0.331 | 0.256 | 0.0357 | 0.0104 | 0.0397 | 0.0045 |
| 3 | 0.520 | 0.124 | 0.623 | 0.304 | 0.0383 | 0.0130 | 0.0744 | 0.0113 |
| 4 | 0.723 | 0.129 | 0.757 | 0.288 | 0.0393 | 0.0113 | 0.0636 | 0.0075 |
| 5 | 0.503 | 0.207 | 0.776 | 0.293 | 0.0480 | 0.0147 | 0.0588 | 0.0090 |

It is observed that the VLCMUFA C24:1 is highest for the group fed Test Diet No. 5.

### Results Neutral Lipids—Skin

The results of the analysis of PUFAs with 22 carbons or more in neutral lipids of skin are shown in Table 12 below, and the results for each fatty acid are shown in FIGS. 75 to 76, wherein

FIG. 75. Content of C24:1 (μg/g tissue) in skin) from mice fed Test Diet 1, 2, 3, 4 and 5.

FIG. 76. Content of C26:1 (μg/g tissue) in skin from mice fed Test Diet 1, 2, 3, 4 and 5.

TABLE 12
Average values of different fatty acids of neutral lipid fraction from each diet group

| | EPA | DPA | DHA | C24:0 | C24:1 | C24:5 | C26:1 | C26:7 | C28:8 |
|---|---|---|---|---|---|---|---|---|---|
| Test | μg/g | μg/g | μg/g | μg/g | μg/g | μg/g | μg/g | μg/g | μg/g |
| Diet | tissue | tissue | tissue | tissue | tissue | tissue | tissue | tissue | tissue |
| 1 | 5.820 | 9.283 | 15.071 | 1.661 | 0.630 | 0.362 | 0.072 | 0.545 | 0.147 |
| 2 | 6.274 | 7.915 | 20.094 | 1.295 | 0.230 | 0.260 | 0.082 | 0.804 | 0.108 |
| 3 | 5.559 | 10.694 | 34.201 | 1.703 | 0.786 | 0.588 | 0.067 | 0.946 | 0.265 |
| 4 | 4.298 | 6.702 | 23.885 | 1.502 | 1.314 | 0.395 | 0.106 | 0.713 | 0.447 |
| 5 | 5.217 | 12.074 | 52.378 | 2.172 | 1.993 | 0.838 | 0.358 | 0.651 | 1.318 |

### Conclusion

The results show that feeding mice with diets with increasing amounts of VLC-fatty acids, lead to an increased concentration of both VLCPUFAs and VLCMUFAs in skin tissue.

### Plasma

The results of the analysis of PUFAs with 22 carbons or more in blood plasma are shown in Table 13 below, and the results for each fatty acid are shown in FIG. 77, wherein

FIG. 77. Content of C24:1 (μg/g blood plasma) from mice fed Test Diet 1, 2, 3 4 and 5.

TABLE 13
Average values of total lipids from each
diet group of different fatty acids

| | C24:1 |
|---|---|
| Diet group | μg/g tissue |
| 1 | 0.00174 |
| 2 | 0.00239 |
| 3 | 0.00185 |
| 4 | 0.00301 |
| 5 | 0.00383 |

### Conclusion

The results show that feeding mice diets with increasing amount of VLC-fatty acids, leads to an increased concentration of VLCMUFAs in blood plasma.

### REFERENCES

1) Folch, J. Lees, M, Sloane Stanley G H. A simple method for the isolation and purification of total lipids from animal tissues. J Biol Chem. 1957; 226 (1):497-509. PMID: 13428781.

2) Vuong T T, Rønning S B, Ahmed T A E, Brathagen K, Host V, Hincke M T, et al. Processed eggshell membrane powder regulates cellular functions and increase MMP-activity important in early wound healing processes. PLoS One. 2018; 13 (8):e0201975. DOI: 10.1371/journal.pone.0201975.

1. A method of treating a disease in a subject in need thereof, the method comprising:
administering to the subject a composition comprising a fatty acid mixture, wherein the fatty acid mixture comprises at least 5% by weight of very long chain fatty acids (VLCFAs) having a chain length of 24 carbon atoms or more, wherein the VLCFAs are isolated from an oil originating from a marine animal or plant, and wherein the composition comprises both omega-3 and/or omega-6 very long chain polyunsaturated fatty acids (VLCPUFAs) and very long chain monounsaturated fatty acids (VLCMUFAs),
wherein the subject has a deficient or abnormal level of VLCFAs present in one or more specific body tissues where the VLCFAs play a role in a disease, and wherein the administered VLCFAs are taken up by one or more of the specific body tissues, thereby providing a positive health effect, optionally, prevention or treatment of the disease.

2. The method of claim 1, wherein the positive health effect is the treatment of the disease, and wherein the disease is related to a deficiency in one or more endogenous elongase systems and/or a reduced ability for endogenic synthesis of VLCFAs.

3. The method of claim 1, wherein the positive health effect is the treatment of the disease, and wherein the disease is related to a deficiency in one or more elongase systems and/or a desaturase system and/or a β-oxidation system.

4. The method of claim 1, wherein the administered VLCFAs are transported to specific body tissues where the administered VLCFAs play a role for a normal tissue function.

5. The method of claim 1, wherein the disease is associated with a deficiency in one or more of the elongase systems ELOVL 1-7.

6. The method of claim 1, wherein the positive health effect is the treatment of the disease, and wherein the treatment is directed towards specific body tissues selected from the group consisting of tissues of the eye, optionally, the eyeball, the retinas, and the meibum; tissues of the sperm and the testes; tissues of the brain and the nervous system; skin, epidermal tissues, epithelial tissues, endothelial tissues, and mucosal membranes/tissues, optionally, tissues of the lung and the respiratory tract; and cardiovascular tissues.

7. The method of claim 1, claim 1, wherein the subject suffers from age-related or hereditary reduced effectiveness of one or more of the body's elongase systems.

8. The method of claim 1, wherein the positive health effect is the prevention of the disease, and wherein, optionally, the prevention comprises maintenance of normal tissue function, or improvement of tissue function, in the specific body tissues where the VLCFAs play a role in the disease to be prevented.

9. The method of claim 1, wherein the positive health effect is the treatment of the disease, and wherein the treatment comprises increased or normalized levels of VLCFAs in the specific body tissues where the VLCFAs play a role in the disease to be treated.

10. The method of claim 1, wherein the disease is selected from the group consisting of: diseases related to eye health; diseases related to male fertility; diseases of the skin, epidermal tissues, epithelial tissues, endothelial tissues, and mucosal tissues/mucous membranes; diseases of the brain and the nervous tissue; cardiovascular diseases; and inflammatory diseases.

11. The method of claim 1, wherein the disease is selected from the group consisting of:
i) eye diseases, optionally, macular degeneration (AMD), diseases caused by diabetic inflammation of the eye, and dominant Stargardt macular dystrophy (STGD3);
ii) diseases related to male fertility, optionally, reduced function and/or viability of the sperm, and a reduced amount of mature sperm cells;
iii) skin, epidermal, epithelial, and endothelial diseases, optionally, dry and wrinkled skin, irritated skin, sore skin, and sensitive skin, reduced ability for wound healing, skin damage from the sun's UV radiation, reduced function of hair follicles, reduced hair health, optionally, risk of hair loss, eczema, psoriasis, acne, and rosacea;
iv) diseases of mucosal tissue/mucous membranes, optionally, lung diseases and diseases of the respiratory tract, optionally, asthma, liver diseases, allergy, and diseases of the urine system and the digestive system;
v) diseases of the brain and the nervous tissue, optionally, the central nervous system, optionally, reduced mental health, demyelinating diseases, optionally, multiple sclerosis, Parkinson's, Schizophrenia, Dementia, Alzheimer's, impaired cognitive function, migraine, seizures, and epilepsy;
vi) inflammatory related diseases, optionally, rheumatoid arthritis, and cardiovascular diseases, optionally, atherosclerosis.

12. The method of claim 1, wherein the composition comprises either of omega-3 and omega-6 VLCPUFAs with more than 6 double bonds.

13. The method of claim 1, wherein the composition comprises at least one of C28:7n3 and C28:8n3.

14. The method of claim 1, wherein the composition further comprises very long chain saturated fatty acids.

15. The method of claim 1, wherein the composition further comprises DHA (C22:6n3) or n3DPA (C22:5n3).

16. The method of claim 1, wherein the composition comprises at least 10% by weight of very long chain mono- and polyunsaturated fatty acids in total.

17. The method of claim 1, wherein the VLCFAs of the composition are isolated from fish oil, mollusc oil, crustacean oil, sea mammal oil, plankton oil, algal oil or microalgal oil.

18. The method of claim 1, wherein the VLFAs of the composition are substantially on the all-cis-form.
